SEQUENCE LISTING (44 sequences)

SEQ ID NO: 1 - protein, 507 aa, Mus musculus
MDKLRVPLWPRVGPLCLLLAGAAWAPSPSLPDPKFESKAALLASRGSEELLCFTQRLEDLVCFWEEAASSGMDFNYSFSYQLEGESRKSCSLHQAPTVRGSVRFWCSLPTADTSSFVPLELQVTEASGSPRYHRIIHINEVVLLDAPAGLLARRAEEGSHVVLRWLPPPGAPMTTHIRYEVDVSAGNRAGGTQRVEVLEGRTECVLSNLRGGTRYTFAVRARMAEPSFSGFWSAWSEPASLLTASDLDPLILTLSLILVLISLLLTVLALLSHRRTLQQKIWPGIPSPESEFEGLFTTHKGNFQLWLLQRDGCLWWSPGSSFPEDPPAHLEVLSEPRWAVTQAGDPGADDEGPLLEPVGSEHAQDTYLVLDKWLLPRTPCSENLSGPGGSVDPVTMDEASETSSCPSDLASKPRPEGTSPSSFEYTILDPSSQLLCPRALPPELPPTPPHLKYLYLVVSDSGISTDYSSGGSQGVHGDSSDGPYSHPYENSLVPDSEPLHPGYVACS

SEQ ID NO: 2 - protein, 483 aa, Mus musculus
APSPSLPDPKFESKAALLASRGSEELLCFTQRLEDLVCFWEEAASSGMDFNYSFSYQLEGESRKSCSLHQAPTVRGSVRFWCSLPTADTSSFVPLELQVTEASGSPRYHRIIHINEVVLLDAPAGLLARRAEEGSHVVLRWLPPPGAPMTTHIRYEVDVSAGNRAGGTQRVEVLEGRTECVLSNLRGGTRYTFAVRARMAEPSFSGFWSAWSEPASLLTASDLDPLILTLSLILVLISLLLTVLALLSHRRTLQQKIWPGIPSPESEFEGLFTTHKGNFQLWLLQRDGCLWWSPGSSFPEDPPAHLEVLSEPRWAVTQAGDPGADDEGPLLEPVGSEHAQDTYLVLDKWLLPRTPCSENLSGPGGSVDPVTMDEASETSSCPSDLASKPRPEGTSPSSFEYTILDPSSQLLCPRALPPELPPTPPHLKYLYLVVSDSGISTDYSSGGSQGVHGDSSDGPYSHPYENSLVPDSEPLHPGYVACS

SEQ ID NO: 3 - protein, 265 aa, Mus musculus
MDKLRVPLWPRVGPLCLLLAGAAWAPSPSLPDPKFESKAALLASRGSEELLCFTQRLEDLVCFWEEAASSGMDFNYSFSYQLEGESRKSCSLHQAPTVRGSVRFWCSLPTADTSSFVPLELQVTEASGSPRYHRIIHINEVVLLDAPAGLLARRAEEGSHVVLRWLPPPGAPMTTHIRYEVDVSAGNRAGGTQRVEVLEGRTECVLSNLRGGTRYTFAVRARMAEPSFSGFWSAWSEPASLLTASGEALVPRGAGGAGPNTRQTP

SEQ ID NO: 4 - protein, 508 aa, Homo sapiens
MDHLGASLWPQVGSLCLLLAGAAWAPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSDLDPLILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 5 - protein, 484 aa, Homo sapiens
APPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSDLDPLILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 6 - protein, 241 aa, Homo sapiens
MDHLGASLWPQVGSLCLLLAGAAWAPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRGTVFLSPDWLSSTRARPHVIYFCLLRVPRPDSAPRWRSWRAAPSVC

SEQ ID NO: 7 - protein, 328 aa, Homo sapiens
MDHLGASLWPQVGSLCLLLAGAAWAPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSDLDPLILTLSLILVVILVLLTVLALLSHRRALKQKIWPGIPSPESEFEGLFTTHKGNFQVGGLVVPSVPGLPCFLQPNCRPL

SEQ ID NO: 8 - protein, 248 aa, Artificial sequence
  Synthetic
APSPSLPDPKFESKAALLASRGSEELLCFTQRLEDLVCFWEEAASSGMDFNYSFSYQLEGESRKSCSLHQAPTVRGSVRFWCSLPTADTSSFVPLELQVTEASGSPRYHRIIHINEVVLLDAPAGLLARRAEEGSHVVLRWLPPPGAPMTTHIRYEVDVSAGNRAGGTQRVEVLEGRTECVLSNLRGGTRYTFAVRARMAEPSFSGFWSAWSEPASLLTASDLDPLILTLSLILVLISLLLTVLALLS

SEQ ID NO: 9 - protein, 225 aa, Artificial sequence
  Synthetic
APSPSLPDPKFESKAALLASRGSEELLCFTQRLEDLVCFWEEAASSGMDFNYSFSYQLEGESRKSCSLHQAPTVRGSVRFWCSLPTADTSSFVPLELQVTEASGSPRYHRIIHINEVVLLDAPAGLLARRAEEGSHVVLRWLPPPGAPMTTHIRYEVDVSAGNRAGGTQRVEVLEGRTECVLSNLRGGTRYTFAVRARMAEPSFSGFWSAWSEPASLLTASDLDP

SEQ ID NO: 10 - protein, 23 aa, Artificial sequence
  Synthetic
LILTLSLILVLISLLLTVLALLS

SEQ ID NO: 11 - protein, 1165 aa, Artificial sequence
  Synthetic
MICQKFCVVLLHWEFIYVITAFNLSYPITPWRFKLSCMPPNSTYDYFLLPAGLSKNTSNSNGHYETAVEPKFNSSGTHFSNLSKTTFHCCFRSEQDRNCSLCADNIEGKTFVSTVNSLVFQQIDANWNIQCWLKGDLKLFICYVESLFKNLFRNYNYKVHLLYVLPEVLEDSPLVPQKGSFQMVHCNCSVHECCECLVPVPTAKLNDTLLMCLKITSGGVIFQSPLMSVQPINMVKPDPPLGLHMEITDDGNLKISWSSPPLVPFPLQYQVKYSENSTTVIREADKIVSATSLLVDSILPGSSYEVQVRGKRLDGPGIWSDWSTPRVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDIKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVIVSWILSPSDYKLMYFIIEWKNLNEDGEIKWLRISSSVKKYYIHDHFIPIEKYQFSLYPIFMEGVGKPKIINSFTQDDIEKHQSDAGLYVIVPVIISSSILLLGTLLISHQRMKKLFWEDVPNPKNCSWAQGLNFQKPETFEHLFIKHTASVTCGPLLLEPETISEDISVDTSWKNKDEMMPTTVVSLLSTTDLEKGSVCISDQFNSVNFSEAEGTEVTYEDESQRQPFVKYATLISNSKPSETGEEQGLINSSVTKCFSSKNSPLKDSFSNSSWEIEAQAFFILSDQHPNIISPHLTFSEGLDELLKLEGNFPEENNDKKSIYYLGVTSIKKRESGVLLTDKSRVSCPFPAPCLFTDIRVLQDSCSHFVENNINLGTSSKKTFASYMPQFQTCSTQTHKIMENKMCDLTV

SEQ ID NO: 12 - protein, 841 aa, Artificial sequence
  Synthetic
FNLSYPITPWRFKLSCMPPNSTYDYFLLPAGLSKNTSNSNGHYETAVEPKFNSSGTHFSNLSKTTFHCCFRSEQDRNCSLCADNIEGKTFVSTVNSLVFQQIDANWNIQCWLKGDLKLFICYVESLFKNLFRNYNYKVHLLYVLPEVLEDSPLVPQKGSFQMVHCNCSVHECCECLVPVPTAKLNDTLLMCLKITSGGVIFQSPLMSVQPINMVKPDPPLGLHMEITDDGNLKISWSSPPLVPFPLQYQVKYSENSTTVIREADKIVSATSLLVDSILPGSSYEVQVRGKRLDGPGIWSDWSTPRVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDIKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVIVSWILSPSDYKLMYFIIEWKNLNEDGEIKWLRISSSVKKYYIHDHFIPIEKYQFSLYPIFMEGVGKPKIINSFTQDDIEKHQSDAGLYVIVPVIISSSILLLGTLLI

SEQ ID NO: 13 - protein, 818 aa, Artificial sequence
  Synthetic
FNLSYPITPWRFKLSCMPPNSTYDYFLLPAGLSKNTSNSNGHYETAVEPKFNSSGTHFSNLSKTTFHCCFRSEQDRNCSLCADNIEGKTFVSTVNSLVFQQIDANWNIQCWLKGDLKLFICYVESLFKNLFRNYNYKVHLLYVLPEVLEDSPLVPQKGSFQMVHCNCSVHECCECLVPVPTAKLNDTLLMCLKITSGGVIFQSPLMSVQPINMVKPDPPLGLHMEITDDGNLKISWSSPPLVPFPLQYQVKYSENSTTVIREADKIVSATSLLVDSILPGSSYEVQVRGKRLDGPGIWSDWSTPRVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDIKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVIVSWILSPSDYKLMYFIIEWKNLNEDGEIKWLRISSSVKKYYIHDHFIPIEKYQFSLYPIFMEGVGKPKIINSFTQDDIEKHQSD

SEQ ID NO: 14 - protein, 23 aa, Artificial sequence
  Synthetic
AGLYVIVPVIISSSILLLGTLLI

SEQ ID NO: 15 - protein, 638 aa, Artificial sequence
  Synthetic
MDLWQLLLTLALAGSSDAFSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIWIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQFTCEEDFYFPWLLIIIFGIFGLTVMLFVFLFSKQQRIKMLILPPVPVPKIKGIDPDLLKEGKLEEVNTILAIHDSYKPEFHSDDSWVEFIELDIDEPDEKTEESDTDRLLSSDHEKSHSNLGVKDGDSGRTSCCEPDILETDFNANDIHEGTSEVAQPQRLKGEADLLCLDQKNQNNSPYHDACPATQQPSVIQAEKNKPQPLPTEGAESTHQAAHIQLSNPSSLSNIDFYAQVSDITPAGSVVLSPGQKNKAGMSQCDMHPEMVSLCQENFLMDNAYFCEADAKKCIPVAPHIKVESHIQPSLNQEDIYITTESLTTAAGRPGTGEHVPGSEMPVPDYTSIHIVQSPQGLILNATALPLPDKEFLSSCGYVSTDQLNKIMP

SEQ ID NO: 16 - protein, 270 aa, Artificial sequence
  Synthetic
FSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIWIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQFTCEEDFYFPWLLIIIFGIFGLTVMLFVFLFS

SEQ ID NO: 17 - protein, 246 aa, Artificial sequence
  Synthetic
FSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIWIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQFTCEEDFY

SEQ ID NO: 18 - protein, 24 aa, Artificial sequence
  Synthetic
FPWLLIIIFGIFGLTVMLFVFLFS

SEQ ID NO: 19 - protein, 1210 aa, Artificial sequence [sequence omitted]

SEQ ID NO: 20 - protein, 644 aa, Artificial sequence
  Synthetic
LEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 21 - protein, 621 aa, Artificial sequence [sequence omitted]

SEQ ID NO: 22 - protein, 23 aa, Artificial sequence
  Synthetic
IATGMVGALLLLLVVALGIGLFM

SEQ ID NO: 23 - protein, 822 aa, Artificial sequence
  Synthetic
MSSWIRWHGPAMARLWGFCWLVVGFWRAAFACPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHLSVYAVVVIASVVGFCLLVMLFLLKLARHSKFGMKGPASVISNDDDSASPLHHISNGSNTPSSSEGGPDAVIIGMTKIPVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHMRKNIKGIHTLLQNLAKASPVYLDILG

SEQ ID NO: 24 - protein, 423 aa, Artificial sequence
  Synthetic
CPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHLSVYAVVVIASVVGFCLLVMLFLL

SEQ ID NO: 25 - protein, 399 aa, Artificial sequence
  Synthetic
CPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREH

SEQ ID NO: 26 - protein, 24 aa, Artificial sequence
  Synthetic
LSVYAVVVIASVVGFCLLVMLFLL

SEQ ID NO: 27 - protein, 427 aa, Artificial sequence
  Synthetic
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNSCKQNKQGANSRPVNQTPPPEGEKLHSDSGISVDSQSLHDQQPHTQTASGQALKGDGGLYSSLPPAKREEVEKLLNGSAGDTWRHLAGELGYQPEHIDSFTHEACPVRALLASWATQDSATLDALLAALRRIQRADLVESLCSESTATSPV

SEQ ID NO: 28 - protein, 244 aa, Artificial sequence
  Synthetic
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAF

SEQ ID NO: 29 - protein, 222 aa, Artificial sequence
  Synthetic
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDN

SEQ ID NO: 30 - protein, 22 aa, Artificial sequence
  Synthetic
LIPVYCSILAAVVVGLVAYIAF

SEQ ID NO: 31 - protein, 248 aa, Artificial sequence
  Synthetic
APSPSLPDPKFESKAALLASRGSEELLCFTQRLEDLVCFWEEAASSGMDFNYSFSYQLEGESRKSCSLHQAPTVRGSVRFWCSLPTADTSSAVPLELQVTEASGSPRYHRIIHINEVVLLDAPAGLLARRAEEGSHVVLRWLPPPGAPMTTHIRYEVDVSAGNRAGGTQRVEVLEGRTECVLSNLRGGTRYTFAVRARMAEPSFSGFWSAWSEPASLLTASDLDPLILTLSLILVLISLLLTVLALLS

SEQ ID NO: 32 - protein, 97 aa, Artificial sequence
  Synthetic
MASRILWHEMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 33 - protein, 108 aa, Artificial sequence
  Synthetic
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 34 - protein, 126 aa, Artificial sequence
  Synthetic
VQLQESGGGLVQAGDSLKLSCEASGDSIGTYVIGWFRQAPGKERIYLATIGRNLVGPSDFYTRYADSVKGRFAVSRDNAKNTVNLQMNSLKPEDTAVYYCAAKTTTWGGNDPNNWNYWGQGTQVTV

SEQ ID NO: 35 - protein, 243 aa, Artificial sequence
  Synthetic
DIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVS

SEQ ID NO: 36 - protein, 251 aa, Artificial sequence
  Synthetic
DIVMTQTAPSVFVTPGESVSISCRSSKSLLHSNGNTYLYWFLQRPGQSPQLLIYRMSNLASGVPDRFSGSGSGTDFTLRISRVEAEDVGVYYCMQHLEYPVTFGAGTKVEIKRGGGGSGGGGSGGGGSGGGGSQVQLQQSGPELVKPGASVKISCKVSGYAISSSWMNWVKQRPGHGLEWIGRIYPGDGDTKYNGKFKDKATLTVDKSSSTAYMQLSSLTSVDSAVYFCARDGYRYYFDYWGQGTSVTVSS

SEQ ID NO: 37 - protein, 249 aa, Artificial sequence
  Synthetic
DIVLTQSPPSLAVSLGQRATISCRASESIDLYGFTFMHWYQQKPGQPPKILIYRASNLESGIPARFSGSGSRTDFTLTINPVEADDVATYYCQQTHEDPYTFGGGTKLEIKRGGGGSGGGGSGGGGSGGGGSQVQLQQSGAELAKPGASVKMSCKTSGYSFSSYWMHWVKQRPGQGLEWIGYINPSTGYTENNQKFKDKVTLTADKSSNTAYMQLNSLTSEDSAVYYCARSGRLYFDVWGAGTTVTVSS

SEQ ID NO: 38 - protein, 253 aa, Artificial sequence
  Synthetic
DIVLTQSPASLAVSLGQRATISCKASQSVDFDGDSYMNWYQQKPGQPPKLLIFAASNLASGIPARLSGSGSGTDFTLNIQPVEEEDAATYYCQQSNEDPYTFGGGTKLEIKGGGGSGGGGSGGGGSGGGGSQVQLQQSGDDLVKPGASVKLSCKASGYTFTTYYINWMRQRPGQGLEWIGRIAPASGTTYSSEMFKDKATLTVDTSSNTAYIQLSSLSSEDSAVYFCARADYGFNSGEAMDYWGQGTSVTVSS

SEQ ID NO: 39 - protein, 111 aa, Artificial sequence
  Synthetic
QSELTQPPSASGTPGQRVTISCSGSSSNIGSNYVYWYQQLPGTAPKLLIYRNNQRPSGVPDRFSGSKSGTSASLAISGLRSEDEADYYCAAWDDSLSAWVFGGGTQLDILG

SEQ ID NO: 40 - protein, 116 aa, Artificial sequence
  Synthetic
QMQLLESGPGLVKPSETLSLTCTVSGGSIWGWIRQPPGKGLEWIGSIYSSGSTYYNPSLKSRVTTSVDTSKNQFSLRLSSVTAADTAVYYCVAWFGDLLSLKGVELWGQGTLVTVS

SEQ ID NO: 41 - protein, 277 aa, Artificial sequence
  Synthetic
NKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 42 - protein, 277 aa, Artificial sequence
  Synthetic
NKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQASTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 43 - protein, 571 aa, Artificial sequence
  Synthetic
LRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 44 - protein, 424 aa, Artificial sequence
  Synthetic
MKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSIPLDQYSPSFPDTRSSTCSSGEDSVFSHEPLPEEPCLPRHPTQLANSGLKRR

(19) United States
(12) Patent Application Publication
(10) Pub. No.: US 2022/0396609 A1
(43) Pub. Date: Dec. 15, 2022

(54) CHIMERIC RECEPTOR SENSORS

(71) Applicant: Senti Biosciences, Inc., South San Francisco, CA (US)

(72) Inventors: Russell Morrison Gordley, San Francisco, CA (US); Daniel Frimannsson, Alameda, CA (US); Michelle Elizabeth Hung, South San Francisco, CA (US)

(21) Appl. No.: 17/771,289

(22) PCT Filed: Oct. 30, 2020
(86) PCT No.: PCT/US20/58118

§ 371 Date: Apr. 22, 2022

Related U.S. Application Data
(60) Provisional application No. 62/929,537, filed on Nov. 1, 2019.

(51) Int. Cl. C07K 14/715; C07K 14/72; C07K 14/71; C07K 14/705; C12N 9/12

(52) U.S. Cl. CPC C07K 14/715; C07K 14/72; C07K 14/71; C07K 14/70571; C12N 9/12; C12Y 207/10001; C07K 2318/20; A61K 38/00

(57) ABSTRACT

Provided herein are generalized extracellular molecule sensors (GEMSs) and polynucleotides encoding the GEMSs. Also provided herein are methods of making and using the GEMSs, such as therapeutic and diagnostic methods.

### CROSS-REFERENCE TO RELATED APPLICATIONS

This application claims the benefit of U.S. Provisional Patent Application No. 62/929,537, filed Nov. 1, 2019, the contents of which are incorporated by reference herein.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 138,967 Byte ASCII (Text) file named “38084-601_ST25.TXT,” created on Oct. 30, 2020.

### BACKGROUND

Mammalian cells programmed to respond to extracellular inputs in a predictable manner have become increasingly important for a wide range of applications, such as cancer immunotherapy, tissue patterning and smart cell implants. The field of programmable receptor engineering is rapidly evolving (See Lim W A & June C H, Cell, 2017, 168:724-740; Brenner M et al., Nat. Chem. Biol., 2017, 13:131-132), but robust sensing of soluble molecules still mostly relies on natural receptors that can be rewired to drive expression of transgenes that have a desired biological function. For example, natural ligand-receptor interactions have been used to engineer designer cells that sense various biomarkers and secrete therapeutic peptides in response. This approach has been used to develop therapeutic cell implants consisting of encapsulated designer cells for the detection and treatment of psoriasis, Graves' disease and metabolic syndrome (See Schukur L et al., Sci. Transl. Med., 2015, 7:318ra201; Saxena P et al., Proc. Natl. Acad. Sci. USA, 2016, 113:1244-1249; Ye H et al., Proc. Natl. Acad. Sci. USA, 2013 110:141-146). Nevertheless, engineering robust input-output relationships in mammalian cells is a laborious iterative process, and many molecules that would be valuable targets for diagnostic or therapeutic purposes do not oftentimes bind to any known naturally occurring receptor. Thus, large groups of potential molecular inputs cannot be readily targeted by this approach. Notably, this includes many synthetic small-molecule compounds, intracellular proteins and extracellular proteins without known signaling function(s).

### SUMMARY

Provided herein are chimeric ligand receptors and related methods.

In one aspect, provided herein is a chimeric ligand receptor comprising a receptor subunit, wherein the receptor subunit comprises a scaffold domain; wherein the scaffold domain comprises an extracellular domain and a transmembrane domain; wherein the extracellular domain is operably linked to a ligand binding domain; wherein the transmembrane domain is operably linked to an intracellular signaling domain; wherein the receptor subunit multimerizes via its scaffold domain in the presence of one or more additional receptor subunits; and wherein the multimerized receptor subunits undergo a conformational reorganization upon ligand binding to the chimeric ligand receptor.

In some embodiments, the chimeric ligand receptor further comprises one or more additional receptor subunits, wherein each additional receptor subunit comprises a scaffold domain; wherein the scaffold domain of each additional receptor subunit comprises an extracellular domain and a transmembrane domain; wherein the extracellular domain of each additional receptor subunit is operably linked to a ligand binding domain; and wherein the transmembrane domain of each additional receptor subunit is operably linked to an intracellular signaling domain. In some embodiments, the receptor subunits multimerize via their scaffold domains to form the chimeric ligand receptor. In some embodiments, the multimerized receptor subunits undergo a conformational reorganization upon ligand binding to the chimeric receptor. In some embodiments, the chimeric ligand binding domains of each receptor subunit bind the same ligand.

In another aspect, provided herein is a chimeric ligand receptor comprising two or more receptor subunits, wherein each receptor subunit comprises a scaffold domain; wherein the receptor subunits multimerize via their scaffold domains to form the chimeric ligand receptor; wherein each scaffold domain comprises an extracellular domain and a transmembrane domain; wherein the extracellular domain is operably linked to a ligand binding domain, wherein the ligand binding domains of each receptor subunit bind the same ligand; wherein the transmembrane domain is operably linked to an intracellular signaling domain; and wherein binding of the ligand induces a conformational reorganization of the multimerized receptor subunits.

In some embodiments, multimerization of the receptor subunits occurs prior to ligand binding. In some embodiments, the multimerized receptor subunits comprise a dimer, a trimer, tetramer, pentamer, or hexamer. In some embodiments, the multimerized receptor subunits comprise a dimer. In some embodiments, the conformational reorganization comprises a rotation of each scaffold domain around its own axis. In some embodiments, the conformational reorganization activates the intracellular signaling domains of each receptor subunit. In some embodiments, the conformational reorganization inhibits the intracellular signaling domains of each receptor subunit.

In some embodiments, the scaffold domain comprises the extracellular domain and transmembrane domain of a cytokine receptor. In some embodiments, the scaffold domain comprises the extracellular domain and transmembrane domain of an erythropoietin receptor (EpoR). In some embodiments, the scaffold domain comprises the extracellular domain and transmembrane domain of a leptin receptor (Ob-R). In some embodiments, the scaffold domain comprises the extracellular domain and transmembrane domain of a growth hormone receptor (GHR). In some embodiments, the scaffold domain comprises the extracellular domain and transmembrane domain of an epidermal growth factor receptor (EGFR). In some embodiments, the scaffold domain is derived from a brain-derived neurotrophic receptor, such as Trk-B or p75^{NTR}. In some embodiments, the scaffold domain is inert to its native ligand. In some embodiments, the scaffold domain is inert to erythropoietin. In some embodiments, the scaffold domain is inert to leptin. In some embodiments, the scaffold domain comprises an extracellular domain and transmembrane domain comprising an amino acid sequence having 90% or greater sequence identity to SEQ ID NO: 8, SEQ ID NO: 12, SEQ ID NO: 16, SEQ ID NO: 20, SEQ ID NO: 24, or SEQ ID NO: 28.

In some embodiments, the extracellular domain, the transmembrane domain, or both the extracellular domain and transmembrane domain of the scaffold domain comprise one or more modifications. In some embodiments, the extracellular domain comprises one or more amino acid substitutions, optionally wherein the extracellular domain comprises an F93A amino acid substitution. In some embodiments, one or more additional amino acid residues are inserted adjacent to the transmembrane domain. In some embodiments, one or more additional amino acid residues are inserted within the transmembrane domain. In some embodiments, the one or more additional amino acid residues are alanine residues. In some embodiments, the transmembrane domain further comprises one, two, three, or four additional alanine residues. In some embodiments, the one or more additional amino acid residues are inserted C-terminal to the transmembrane domain.

In some embodiments, the ligand binding domain is linked to the extracellular domain through an extracellular linker region. In some embodiments, the extracellular linker region comprises one or more amino acid residues, optionally wherein the one or more amino acid residues comprise amino acids residues Serine-Glycine-Glutamic acid-Phenylalanine.

In some embodiments, the ligand binding domain does not bind a native ligand of the scaffold domain. In some embodiments, the ligand binding domain does not bind erythropoietin. In some embodiments, the ligand binding domain does not bind leptin. In some embodiments, the ligand binding domain is not derived from a cytokine receptor.

In some embodiments, the ligand binding domain binds to a ligand selected from the group consisting of a protein complex, a protein, a peptide, a nucleic acid, a small molecule, and a chemical agent. In some embodiments, the ligand is selected from the group consisting of an antigen, a cytokine, a survival factor, a chemokine, a hormone, a transmitter, a growth factor, extracellular matrix, and a death factor. In some embodiments, the ligand binding domain binds to a ligand displayed on a surface or expressed on a cell surface. In some embodiments, the ligand expressed on a cell surface is a tumor-associated antigen. In some embodiments, the tumor-associated antigen is selected from the group consisting of MS4A3, VSTIM1, LAT2, MLC1, CD131, GAPT, PRAM1, SLC22A16, SLC17A9, SPNS3, FLT3, CD33, CLEC12A, ADGRE2, IL3RA, CD117, CD93, IL1RAP, CD244, CCR1, LILRB2, PIEZO1, CD38, EMB, MYADM, LILRA2, CD300LF, CD70, 5T4, ADAM9, AFP, AXL, B7-H3, B7-H4, B7-H6, C4.4, CA6, Cadherin 3, Cadherin 6, CCR4, CD123, CD133, CD138, CD142, CD166, CD25, CD30, CD352, CD37, CD38, CD44, CD56, CD66e, CD71, CD74, CD79b, CD80, CEA, CEACAM5, Claudin18.2, cMet, CSPG4, CTLA, DLK1, DLL3, DR5, EGFR, ENPP3, EpCAM, EphA2, Ephrin A4, ETBR, FGFR2, FGFR3, FRalpha, FRb, GCC, GD2, GFRa4, gpA33, GPC3, gpNBM, GPRC5, HER2, IL-13R, IL-13Ra2, IL-8, IL-15, IL1RAP, Integrin aV, KIT, LiCAM, LAMP1, Lewis Y, LeY, LIV-1, LRRC, LY6E, MCSP, Mesothelin, MUC1, MUC16, MUC1C, NaPi2B, Nectin 4, NKG2D, NOTCH3, NY ESO 1, Ovarin, P-cadherin, pan-Erb2, PSCA, PSMA, PTK7, ROR1, S Aures, SCT, SLAMF7, SLITRK6, SSTR2, STEAP1, Survivin, TDGF1, TIM1, TROP2, and WT1. In some embodiments, the ligand binding domain binds to a soluble ligand. In some embodiments, the soluble ligand is a secreted cytokine. In some embodiments, the secreted cytokine is an immunosuppressive cytokine. In some embodiments, the secreted cytokine is selected from the group consisting of TGFbeta, IL-10, IL-6, IL-23, IL-8, CCL2, CXCL12, CXCL8, CXCL1, CXCL2, CXCL3, CXCL8, CXCL13, CCL5, CCL17, and CCL22. In some embodiments, the ligand binding domain binds to an antigen.

In some embodiments, the ligand binding domain comprises an antibody, or antigen-binding fragment thereof. In some embodiments, the ligand binding domain comprises a single chain variable fragment (scFv), or a single-domain antibody (sdAb). In some embodiments, each of the ligand binding domains comprises a single chain variable fragment (scFv), optionally wherein each scFv specifically binds to a distinct epitope of the antigen. In some embodiments, the ligand binding domains of each receptor subunit are distinct from one another. In some embodiments, the chimeric ligand receptor comprises two ligand binding domains, and wherein one ligand binding domain comprises an immunoglobulin heavy chain variable domain (V_{H}) and the second ligand binding domain comprises an immunoglobulin light chain variable domain (V_{L}). In some embodiments, the ligand binding domains of each receptor subunit are the same as one another. In some embodiments, the antibody, or antigen-binding fragment thereof, is a single-domain VHH camelid antibody.

In some embodiments, the intracellular signaling domain is inert to native ligand binding of the scaffold domain. In some embodiments, the intracellular signaling domain does not comprise an endogenous intracellular signaling domain of the scaffold domain. In some embodiments, the intracellular signaling domain does not comprise an endogenous erythropoietin receptor (EpoR) intracellular signaling domain. In some embodiments, the intracellular signaling domain does not comprise an endogenous leptin receptor (Ob-R) intracellular signaling domain. In some embodiments, the intracellular signaling domain does not comprise an endogenous cytokine receptor intracellular signaling domain.

In some embodiments, the intracellular signaling domain induces downstream signaling via a JAK/STAT (Janus kinase/signal transducer and activator of transcription) signaling pathway, a MAPK (mitogen-activated protein kinase) signaling pathway, a PLCG (phospholipase C gamma) signaling pathway, or a PI3K/Akt (phosphatidylinositol 3-kinase/protein kinase B) signaling pathway. In some embodiments, the intracellular signaling domain is selected from the group consisting of an intracellular signal transduction domain of IL-6RB (interleukin 6 receptor B), an intracellular signal transduction domain of FGFR1 (fibroblast growth factor receptor 1), and an intracellular signal transduction domain of VEGFR2 (vascular endothelial growth factor receptor 2). In some embodiments, the intracellular signaling domain is an intracellular signal transduction domain of IL-6RB and induces downstream signaling via the JAK/STAT signaling pathway. In some embodiments, the intracellular signaling domain is an intracellular signal transduction domain of FGFR1 and induces downstream signaling via the MAPK signaling pathway. In some embodiments, the intracellular signaling domain is an intracellular signal transduction domain of VEGFR2 and induces downstream signaling via the PLCG signaling pathway. In some embodiments, the intracellular signaling domain is an intracellular signal transduction domain of VEGFR2 and induces downstream signaling via the PI3K/Akt signaling pathways. In some embodiments, the intracellular signaling domain is an intracellular signal transduction domain derived from an IL-2 signaling pathway, an IL-7 signaling pathway, or an IL-15 signaling pathway.

In some embodiments, the intracellular signaling domain comprises one or more modifications that modulate signaling activity of the intracellular signaling domain, optionally wherein the one or more modifications are one or more amino acid substitutions.

In some embodiments, the scaffold domain comprises the extracellular domain and transmembrane domain of an erythropoietin receptor (EpoR) or an extracellular domain and transmembrane domain comprising an amino acid sequence having 90% or greater sequence identity to SEQ ID NO: 8, wherein the extracellular domain comprises an F93A amino acid substitution, wherein the scaffold domain is inert to erythropoietin; wherein the ligand binding domain does not bind erythropoietin; and wherein the intracellular signaling domain does not comprise an endogenous erythropoietin receptor (EpoR) intracellular signaling domain. In some embodiments, the ligand binding domain comprises a single-domain VHH camelid antibody. In some embodiments, the ligand binding domain binds rapamycin, RR120, nicotine, SunTag, or PSA (prostate-specific antigen). In some embodiments, the ligand binding domain comprises an antibody or antigen-binding fragment targeting rapamycin, RR120, nicotine, SunTag, or PSA (prostate-specific antigen). In some embodiments, the intracellular signaling domain comprises an intracellular signal transduction domain of IL-6RB (interleukin 6 receptor B), an intracellular signal transduction domain of FGFR1 (fibroblast growth factor receptor 1), or an intracellular signal transduction domain of VEGFR2 (vascular endothelial growth factor receptor 2). In some embodiments, the intracellular signaling domain induces downstream signaling via a JAK/STAT (Janus kinase/signal transducer and activator of transcription) signaling pathway, a MAPK (mitogen-activated protein kinase) signaling pathway, a PLCG (phospholipase C gamma) signaling pathway, or a PI3K/Akt (phosphatidylinositol 3-kinase/protein kinase B) signaling pathway. In some embodiments, the intracellular signaling domain comprises an intracellular signal transduction domain of IL-6RB (interleukin 6 receptor B). In some embodiments, the intracellular signaling domain induces downstream signaling via a JAK/STAT (Janus kinase/signal transducer and activator of transcription) signaling pathway.

In another aspect, provided herein is an isolated polynucleotide or a set of isolated polynucleotides encoding the chimeric ligand receptor. In some embodiments, the isolated polynucleotide or set of isolated polynucleotides comprises the cDNA of the chimeric ligand receptor

In another aspect, provided herein is an isolated polynucleotide or a set of isolated polynucleotides comprising a nucleic acid sequence encoding a chimeric ligand receptor subunit, wherein the receptor subunit comprises a scaffold domain capable of multimerizing the receptor subunit in the presence of one or more additional receptor subunits and wherein the receptor subunit is capable of undergoing a conformational reorganization induced by ligand binding when multimerized; wherein the scaffold domain comprises an extracellular domain and a transmembrane domain; wherein the extracellular domain is operably linked to a ligand binding domain; and wherein the transmembrane domain is operably linked to an intracellular signaling domain.

In some embodiments, the isolated polynucleotide or set of isolated polynucleotides further comprises one or more nucleic acid sequences, wherein each additional nucleic acid sequence encoding an additional chimeric ligand receptor subunit; wherein each additional receptor subunit comprises a scaffold domain; wherein the scaffold domain of each additional receptor subunit comprises an extracellular domain and a transmembrane domain; wherein the extracellular domain of each additional receptor subunit is operably linked to a ligand binding domain; and wherein the transmembrane domain of each additional receptor subunit is operably linked to an intracellular signaling domain. In some embodiments, the receptor subunits multimerize via their scaffold domains to form the chimeric ligand receptor. In some embodiments, the multimerized receptor subunits undergo a conformational reorganization upon ligand binding to the chimeric receptor. In some embodiments, the chimeric ligand binding domains of each receptor subunit bind the same ligand.

In another aspect, provided herein is an isolated polynucleotide or a set of isolated polynucleotides comprising two or more nucleic acid sequences, wherein each nucleic acid sequence encoding a chimeric ligand receptor subunit; wherein each receptor subunit comprises a scaffold domain; wherein the receptor subunits multimerize via their scaffold domains to form the chimeric ligand receptor; wherein each scaffold domain comprises an extracellular domain and a transmembrane domain; wherein the extracellular domain is operably linked to a ligand binding domain, wherein the ligand binding domains of each receptor subunit bind the same ligand; wherein the transmembrane domain is operably linked to an intracellular signaling domain; and wherein binding of the ligand induces a conformational reorganization of the multimerized receptor subunits.

In some embodiments, multimerization of the receptor subunits occurs prior to ligand binding. In some embodiments, the multimerized receptor subunits comprise a dimer, a trimer, tetramer, pentamer, or hexamer. In some embodiments, the multimerized receptor subunits comprise a dimer. In some embodiments, the conformational reorganization comprises a rotation of each scaffold domain around its own axis. In some embodiments, the conformational reorganization activates the intracellular signaling domains of each receptor subunit. In some embodiments, the conformational reorganization inhibits the intracellular signaling domains of each receptor subunit.

In some embodiments, the scaffold domain comprises the extracellular domain and transmembrane domain of a cytokine receptor. In some embodiments, the scaffold domain comprises the extracellular domain and transmembrane domain of an erythropoietin receptor (EpoR). In some embodiments, the scaffold domain comprises the extracellular domain and transmembrane domain of a leptin receptor (Ob-R). In some embodiments, the scaffold domain comprises the extracellular domain and transmembrane domain of a growth hormone receptor (GHR). In some embodiments, the scaffold domain comprises the extracellular domain and transmembrane domain of an epidermal growth factor receptor (EGFR). In some embodiments, the scaffold domain is derived from a brain-derived neurotrophic receptor, such as Trk-B or p75^{NTR}. In some embodiments, the scaffold domain is inert to its native ligand. In some embodiments, the scaffold domain is inert to erythropoietin. In some embodiments, the scaffold domain is inert to leptin. In some embodiments, the scaffold domain comprises an extracellular domain and transmembrane domain comprising an amino acid sequence having 90% or greater sequence identity to SEQ ID NO: 8,SEQ ID NO: 12, SEQ ID NO: 16, SEQ ID NO: 20, SEQ ID NO: 24, or SEQ ID NO: 28.

In some embodiments, the extracellular domain, the transmembrane domain, or both the extracellular domain and transmembrane domain of the scaffold domain comprise one or more modifications. In some embodiments, the extracellular domain comprises one or more amino acid substitutions, optionally wherein the extracellular domain comprises an FP93A amino acid substitution. In some embodiments, one or more additional amino acid residues are inserted adjacent to the transmembrane domain. In some embodiments, one or more additional amino acid residues are inserted within the transmembrane domain. In some embodiments, the one or more additional amino acid residues are alanine residues. In some embodiments, the transmembrane domain further comprises one, two, three, or four additional alanine residues. In some embodiments, the one or more additional amino acid residues are inserted C-terminal to the transmembrane domain.

In some embodiments, the ligand binding domain is linked to the extracellular domain through an extracellular linker region. In some embodiments, the extracellular linker region comprises one or more amino acid residues, optionally wherein the one or more amino acid residues comprise amino acids residues Serine-Glycine-Glutamic acid-Phenylalanine.

In some embodiments, the ligand binding domain does not bind a native ligand of the scaffold domain. In some embodiments, the ligand binding domain does not bind erythropoietin. In some embodiments, the ligand binding domain does not bind leptin. In some embodiments, the ligand binding domain is not derived from a cytokine receptor.

In some embodiments, the ligand binding domain binds to a ligand selected from the group consisting of a protein complex, a protein, a peptide, a nucleic acid, a small molecule, and a chemical agent. In some embodiments, the ligand is selected from the group consisting of an antigen, a cytokine, a survival factor, a chemokine, a hormone, a transmitter, a growth factor, extracellular matrix, and a death factor. In some embodiments, the ligand binding domain binds to a ligand displayed on a surface or expressed on a cell surface. In some embodiments, the ligand binding domain binds to a soluble ligand. In some embodiments, the ligand binding domain binds to an antigen.

In some embodiments, the ligand binding domain comprises an antibody, or antigen-binding fragment thereof. In some embodiments, the ligand binding domain comprises a single chain variable fragment (scFv), or a single-domain antibody (sdAb). In some embodiments, each of the ligand binding domains comprises a single chain variable fragment (scFv), optionally wherein each scFv specifically binds to a distinct epitope of the antigen. In some embodiments, the ligand binding domains of each receptor subunit are distinct from one another. In some embodiments, the chimeric ligand receptor comprises two ligand binding domains, and wherein one ligand binding domain comprises an immunoglobulin heavy chain variable domain (V_{H}) and the second ligand binding domain comprises an immunoglobulin light chain variable domain (V_{L}). In some embodiments, the ligand binding domains of each receptor subunit are the same as one another. In some embodiments, the antibody, or antigen-binding fragment thereof, is a single-domain VHH camelid antibody.

In some embodiments, the intracellular signaling domain is inert to native ligand binding of the scaffold domain. In some embodiments, the intracellular signaling domain does not comprise an endogenous intracellular signaling domain of the scaffold domain. In some embodiments, the intracellular signaling domain does not comprise an endogenous erythropoietin receptor (EpoR) intracellular signaling domain. In some embodiments, the intracellular signaling domain does not comprise an endogenous leptin receptor (Ob-R) intracellular signaling domain. In some embodiments, the intracellular signaling domain does not comprise an endogenous growth hormone receptor (GHR) intracellular signaling domain. In some embodiments, the intracellular signaling domain does not comprise an endogenous epidermal growth factor receptor (EGFR) intracellular signaling domain. In some embodiments, the intracellular signaling domain does not comprise an endogenous epidermal growth factor receptor (EGFR) intracellular signaling domain. In some embodiments, the intracellular signaling domain does not comprise an endogenous Trk-B intracellular signaling domain. In some embodiments, the intracellular signaling domain does not comprise an endogenous p75NTR intracellular signaling domain. In some embodiments, the intracellular signaling domain does not comprise an endogenous cytokine receptor intracellular signaling domain.

In some embodiments, the intracellular signaling domain induces downstream signaling via a JAK/STAT (Janus kinase/signal transducer and activator of transcription) signaling pathway, a MAPK (mitogen-activated protein kinase) signaling pathway, a PLCG (phospholipase C gamma) signaling pathway, or a PI3K/Akt (phosphatidylinositol 3-kinase/protein kinase B) signaling pathway. In some embodiments, the intracellular signaling domain is selected from the group consisting of an intracellular signal transduction domain of IL-6RB (interleukin 6 receptor B), an intracellular signal transduction domain of FGFR1 (fibroblast growth factor receptor 1), and an intracellular signal transduction domain of VEGFR2 (vascular endothelial growth factor receptor 2). In some embodiments, the intracellular signaling domain is an intracellular signal transduction domain of IL-6RB and induces downstream signaling via the JAK/STAT signaling pathway. In some embodiments, the intracellular signaling domain is an intracellular signal transduction domain of FGFR1 and induces downstream signaling via the MAPK signaling pathway. In some embodiments, the intracellular signaling domain is an intracellular signal transduction domain of VEGFR2 and induces downstream signaling via the PLCG signaling pathway. In some embodiments, the intracellular signaling domain is an intracellular signal transduction domain of VEGFR2 and induces downstream signaling via the PI3K/Akt signaling pathways. In some embodiments, the intracellular signaling domain is an intracellular signal transduction domain derived from an IL-2 signaling pathway, an IL-7 signaling pathway, or an IL-15 signaling pathway

In some embodiments, the intracellular signaling domain comprises one or more modifications that modulate signaling activity of the intracellular signaling domain, optionally wherein the one or more modifications are one or more amino acid substitutions.

In some embodiments, the scaffold domain comprises the extracellular domain and transmembrane domain of an erythropoietin receptor (EpoR) or an extracellular domain and transmembrane domain comprising an amino acid sequence having 90% or greater sequence identity to SEQ ID NO: 8, wherein the extracellular domain comprises an F93A amino acid substitution, wherein the scaffold domain is inert to erythropoietin; wherein the ligand binding domain does not bind erythropoietin; and wherein the intracellular signaling domain does not comprise an endogenous erythropoietin receptor (EpoR) intracellular signaling domain. In some embodiments, the ligand binding domain comprises a single-domain VHH camelid antibody. In some embodiments, the ligand binding domain binds rapamycin, RR120, nicotine, SunTag, or PSA (prostate-specific antigen). In some embodiments, the ligand binding domain comprises an antibody or antigen-binding fragment targeting rapamycin, RR120, nicotine, SunTag, or PSA (prostate-specific antigen). In some embodiments, the intracellular signaling domain comprises an intracellular signal transduction domain of IL-6RB (interleukin 6 receptor B), an intracellular signal transduction domain of FGFR1 (fibroblast growth factor receptor 1), or an intracellular signal transduction domain of VEGFR2 (vascular endothelial growth factor receptor 2). In some embodiments, the intracellular signaling domain induces downstream signaling via a JAK/STAT (Janus kinase/signal transducer and activator of transcription) signaling pathway, a MAPK (mitogen-activated protein kinase) signaling pathway, a PLCG (phospholipase C gamma) signaling pathway, or a PI3K/Akt (phosphatidylinositol 3-kinase/protein kinase B) signaling pathway. In some embodiments, the intracellular signaling domain comprises an intracellular signal transduction domain of IL-6RB (interleukin 6 receptor B). In some embodiments, the intracellular signaling domain induces downstream signaling via a JAK/STAT (Janus kinase/signal transducer and activator of transcription) signaling pathway.

In another aspect, provided herein is a vector or a set of vectors comprising the polynucleotide or set of polynucleotides.

In another aspect, provided herein is a genetically engineered cell comprising the polynucleotide or set of polynucleotides or the vector or set of vectors.

In another aspect, provided herein is a genetically engineered cell expressing the chimeric ligand receptor.

In some embodiments, the cell further comprises an engineered transgene, wherein the transgene comprises a synthetic promoter operably linked to a polynucleotide comprising a nucleic acid sequence encoding a target product. In some embodiments, the synthetic promoter is responsive to intracellular signaling from the chimeric ligand receptor. In some embodiments, the target product is selected from the group consisting of a therapeutic molecule, a prophylactic molecule, and a diagnostic molecule. In some embodiments, the target product is glucagon-like peptide 1. In some embodiments, the target product is a chimeric antigen receptor (CAR). In some embodiments, the target product is a proinflammatory cytokine. In some embodiments, the proinflammatory cytokine is selected from the group consisting of IL-2, IL-7, IL-12, IL-15, IL-18, and IL-21.

In some embodiments, the cell further expresses one or more additional chimeric ligand receptors. In some embodiments, the chimeric ligand receptors are each distinct from one another. In some embodiments, the ligand binding domains of each chimeric ligand receptor bind a different ligand.

In another aspect, provided herein is a genetically engineered cell expressing two or more chimeric ligand receptors. In some embodiments, the chimeric ligand receptors are each distinct from one another. In some embodiments, the ligand binding domains of each chimeric ligand receptor bind a different ligand. In some embodiments, the cell further comprises two or more engineered transgenes, wherein each transgene comprises a synthetic promoter operably linked to a polynucleotide comprising a nucleic acid sequence encoding a target product. In some embodiments, each synthetic promoter is responsive to intracellular signaling from a distinct chimeric ligand receptor from the two or more chimeric ligand receptors expressed on the cell. In some embodiments, each target product is independently selected from the group consisting of a therapeutic molecule, a prophylactic molecule, and a diagnostic molecule.

In some embodiments, the cell is a mammalian cell. In some embodiments, the mammalian cell is a stem cell or a neuronal cell. In some embodiments, the stem cell is selected from the group consisting of an adult stem cell, an iPS cell, a bone marrow stem cell, a peripheral blood stem cell, and a mesenchymal stem cell (MSC). In some embodiments, the mammalian cell is an immune cell. In some embodiments, the immune cell is selected from the group consisting of a T cell, a B cell, an NK cell, a monocyte, a macrophage, an innate lymphoid cell, a mast cell, an eosinophil, a basophil, a neutrophil, and a dendritic cell. In some embodiments, the immune cell is a T cell. In some embodiments, the T cell is selected from the group consisting of a helper T cell, a cytotoxic T cell, a memory T cell, a regulatory T cell, a natural killer T cell, and a gamma delta T cell.

In another aspect, provided herein is a method comprising contacting the chimeric ligand receptor or the genetically engineered cell with a biological tissue or biological fluid. In some embodiments, the biological tissue or biological fluid is in a subject or is obtained from a subject. In some embodiments, the subject has been diagnosed with, is at risk of developing, or is suspected of having a medical condition, optionally wherein the medical condition is a cancer or inflammatory condition.

In another aspect, provided herein is a method of activating a signaling pathway. The method comprises contacting the chimeric ligand receptor or the genetically engineered cell with a cognate ligand under conditions suitable for the chimeric ligand receptor to bind the cognate ligand, wherein binding of the cognate ligand with the chimeric ligand receptor induces a conformational reorganization of the multimerized scaffold domains that activates the intracellular signaling domains. In some embodiments, the method further comprises administering the cognate ligand to a surface of a cell.

In another aspect, provided herein is a method of producing a genetically engineered cell expressing a chimeric ligand receptor. The method comprises synthesizing a chimeric ligand receptor expression vector encoding a chimeric ligand receptor comprising a scaffold domain capable of multimerizing and comprising an extracellular domain and a transmembrane domain, a ligand binding domain operably linked to the extracellular binding domain of the scaffold domain, and an intracellular signaling domain operably linked to the transmembrane domain of the scaffold domain, by fusing a first nucleic acid encoding the ligand binding domain to a second nucleic acid encoding the scaffold domain, and fusing the second nucleic acid with a third nucleic acid encoding the intracellular signaling domain; transfecting the chimeric ligand receptor expression vector into a cell; and inducing expression of the chimeric ligand receptor in the cell.

In another aspect, provided herein is a method of producing a genetically engineered cell expressing a chimeric ligand receptor. The method comprises transfecting the isolated polynucleotide or set of isolated polynucleotides or the vector or set of vectors into a cell; and inducing expression of the chimeric ligand receptor in the cell. In some embodiments, the method further comprises transfecting into the cell an isolated polynucleotide comprising a synthetic promoter operably linked to a nucleic acid sequence encoding a target product. In some embodiments, the synthetic promoter is responsive to intracellular signaling from the chimeric ligand receptor. In some embodiments, inducing expression of the chimeric ligand receptor comprises culturing the cell under conditions suitable for the cell to express the chimeric ligand receptor on a cell membrane of the cell.

In another aspect, provided herein is a method comprising transfecting a cell with the polynucleotide or set of isolated polynucleotides or vector or set of vectors, optionally wherein the cell is a mammalian cell, and optionally wherein the mammalian cell is an immune cell.

In another aspect, provided herein is a kit comprising a polynucleotide comprising a nucleic acid sequence encoding at least one chimeric ligand receptor. In some embodiments, the kit further comprises at least one engineered transgene comprising a synthetic promoter operably linked to a polynucleotide comprising a nucleic acid sequence encoding a target product.

### BRIEF DESCRIPTION OF THE DRAWINGS

These and other features, aspects, and advantages of the present disclosure will become better understood with regard to the following description, and accompanying drawings.

FIG. 1 shows a schematic of the mechanism of chimeric receptor activation. In the absence of input, the receptor is locked in an inactive conformation. Binding of ligand facilitates interaction of intracellular domains for downstream signaling.

FIG. 2 shows a schematic of an example of a generalized extracellular molecule sensor (GEMS) platform. The input molecules rapamycin, RR120, nicotine, SunTag and PSA cover a wide range of molecular weights and may be chosen as inputs to verify the generality of the GEMS platform. The affinity domains may dimerize by different mechanisms to activate the receptor. Rapamycin-induced FRB/FKBP heterodimerization may be used for initial characterization of the system. The camelid heavy chain antibody VHH_{A52} forms homodimers in the presence of RR120. The variable chains of the nicotine antibody Nic12 can be fused separately in the receptor framework for heterodimeric receptors based on nicotine-induced stabilization of heavy and light chain interactions. The anti-GCN4 scFv can bind epitopes of a SunTag for homodimeric receptors. Two different scFvs can bind to distinct epitopes of PSA for heterodimeric receptors. Extracellular and transmembrane domains of EpoR cluster as preformed dimers that inhibit downstream signaling in the absence of ligand but can be activated by the dimerization of extracellular domains. Ligand-induced changes of the orientation of intracellular signal transduction domains may be used to activate four different dimerization-dependent signaling pathways. Black arrows show the main signaling pathways of indicated GEMS devices. Dashed arrows exemplify possible activation of additional signaling pathways. All pathways may be been rewired for transgene expression. A STAT3, NF-κB or NFAT responsive minimal promoter may be used for readout of JAK/STAT, PI3K/Akt or PLCG signaling, respectively. At the end of the MAPK signaling cascade, ERK phosphorylates a TetR-Elk1 fusion protein that can activate transcription from a reporter plasmid. Exchange of the reporter protein SEAP for expression of other proteins may be used in various applications, such as therapeutic peptide expression in response to disease markers. The amino acid sequences of various components of these chimeric receptors are set forth in SEQ ID NOs: 31-44.

FIG. 3 shows that IL6-GEMS_{RR120} signals via the IL6 pathway, activating a reporter specific for IL6 signaling. Graph show the mean±s.d. as a bar diagram.

### Definitions

Unless otherwise defined, all terms of art, notations and other scientific terminology used herein are intended to have the meanings commonly understood by those of skill in the art. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, N.Y. As appropriate, procedures involving the use of commercially available kits and reagents are generally carried out in accordance with manufacturer-defined protocols and conditions unless otherwise noted.

As used herein, the singular forms “a,” “an,” and “the” include the plural referents unless the context clearly indicates otherwise. The terms “include,” “such as,” and the like are intended to convey inclusion without limitation, unless otherwise specifically indicated.

As used herein, the term “comprising” also specifically includes embodiments “consisting of” and “consisting essentially of” the recited elements, unless specifically indicated otherwise.

The term “about” indicates and encompasses an indicated value and a range above and below that value. In certain embodiments, the term “about” indicates the designated value ±10%, ±5%, or ±1%. In certain embodiments, where applicable, the term “about” indicates the designated value(s)±one standard deviation of that value(s).

The term “Erythropoietin Receptor (EpoR)” refers a member of the cytokine receptor family. It is encoded by the EPOR gene in humans. EpoR pre-exists as dimers which upon binding of a 30 kDa ligand erythropoietin (Epo), changes its homodimerized conformation. These conformational changes result in the autophosphorylation of Jak2 kinases that are pre-associated with the receptor (i.e., EpoR does not possess intrinsic kinase activity and depends on Jak2 activity). One well-established function of EpoR is to promote proliferation and rescue of erythroid (red blood cell) progenitors from apoptosis. An exemplary RefSeq accession number for human EpoR precursor is NP_000112.1 as shown on the NCBI website as of Oct. 1, 2018. An exemplary RefSeq accession number for mouse EpoR precursor is NP_034279.3 as shown on the NCBI website as of Oct. 1, 2018. Exemplary amino acid sequences for mouse EpoR precursor, mature EpoR, and EpoR isoform EpoR-S and human EpoR precursor, mature EpoR, and EpoR isoforms EpoR-S and EpoR-T are shown below.

| Name | Amino Acid Sequence |
|---|---|
| EpoR | MDKLRVPLWPRVGPLCLLLAGAAWAPSPSLPDPKFESKAA |
| precursor | LLASRGSEELLCFTQRLEDLVCFWEEAASSGMDFNYSFSY |
| (mouse) | QLEGESRKSCSLHQAPTVRGSVRFWCSLPTADTSSFVPLE |
| | LQVTEASGSPRYHRIIHINEVVLLDAPAGLLARRAEEGSH |
| | VVLRWLPPPGAPMTTHIRYEVDVSAGNRAGGTQRVEVLEG |
| | RTECVLSNLRGGTRYTFAVRARMAEPSFSGFWSAWSEPAS |
| | LLTASDLDPLILTLSLILVLISLLLTVLALLSHRRTLQQK |
| | IWPGIPSPESEFEGLFTTHKGNFQLWLLQRDGCLWWSPGS |
| | SFPEDPPAHLEVLSEPRWAVTQAGDPGADDEGPLLEPVGS |
| | EHAQDTYLVLDKWLLPRTPCSENLSGPGGSVDPVTMDEAS |
| | ETSSCPSDLASKPRPEGTSPSSFEYTILDPSSQLLCPRAL |
| | PPELPPTPPHLKYLYLVVSDSGISTDYSSGGSQGVHGDSS |
| | DGPYSHPYENSLVPDSEPLHPGYVACS |
| | (SEQ ID NO: 1) |
| Mature | APSPSLPDPKFESKAALLASRGSEELLCFTQRLEDLVCFW |
| EpoR | EEAASSGMDFNYSFSYQLEGESRKSCSIHQAPTVRGSVRF |
| (mouse) | WCSLPTADTSSFVPLELQVTEASGSPRYHRIIHINEVVLL |
| | DAPAGLLARRAEEGSHVVLRWLPPPGAPMTTHIRYEVDVS |
| | AGNRAGGTQRVEVLEGRTECVLSNLRGGTRYTFAVRARMA |
| | EPSFSGFWSAWSEPASLLTASDLDPLILTLSLILVLISLL |
| | LTVTALLSHRRTLQQKIWPGIPSPESEFEGLFTTHKGNFQ |
| | LWLLQRDGCLWWSPGSSFPEDPPAHLEVLSEPRWAVTQAG |
| | DPGADDEGPLLEPVGSEHAQDTYLVLDKWLLPRTPCSENL |
| | SGPGGSVDPVTMDEASETSSCPSDLASKPRPEGTSPSSFE |
| | YTILDPSSQLLCPRALPPELPPTPPHLKYLYLVVSDSGIS |
| | TDYSSGGSQGVHGDSSDGPYSHPYENSLVPDSEPLHPGYV |
| | ACS |
| | (SEQ ID NO: 2) |
| Isoform | MDKLRVPLWPRVGPLCLLLAGAAWAPSPSLPDPKFESKAA |
| EpoR-S; | LLASRGSEELLCFTQRLEDLVCFWEEAASSGMDFNYSFSY |
| soluble | QLEGESRKSCSLHQAPTVRGSVRFWCSLPTADTSSFVPLE |
| form | LQVTEASGSPRYHRIIHINEVVLLDAPAGLLARRAEEGSH |
| (mouse) | VVLRWLPPPGAPMTTHIRYEVDVSAGNRAGGTQRVEVLEG |
| | RTECVLSNLRGGTRYTFAVRARMAEPSFSGFWSAWSEPAS |
| | LLTASGEALVPRGAGGAGPNTRQTP |
| | (SEQ ID NO: 3) |
| EpoR | MDHLGASLWPQVGSLCLLLAGAAWAPPPNLPDPKFESKAA |
| precursor | LLAARGPEELLCFTERLEDLVCFWEEAASAGVGPGNYSFS |
| (human) | YQLEDEPWKLCRLHQAPTARGAVRFWCSLPTADTSSFVPL |
| | ELRVTAASGAPRYHRVIHINEVVLLDAPVGLVARLADESG |
| | HVVLRWLPPPETPMTSHIRYEVDVSAGNGAGSVQRVEILE |
| | GRTECVLSNLRGRTRYTFAVRARMAEPSFGGFWSAWSEPV |
| | SLLTPSDLDPLILTLSLILVVILVLLTVLALLSHRRALKQ |
| | KIWPGIPSPESEFEGLFTTHKGNFQLWLYQNDGCLWWSPC |
| | TPFTEDPPASLEVLSERCWGTMQAVEPGTDDEGPLLEPVG |
| | SEHAQDTYLVLDKWLLPRNPPSEDLPGPGGSVDIVAMDEG |
| | SEASSCSSALASKPSPEGASAASFEYTILDPSSQLLRPWT |
| | LCPELPPTPPHLKYLYLVVSDSGISTDYSSGDSQGAQGGL |
| | SDGPYSNPYENSLIPAAEPLPPSYVACS |
| | (SEQ ID NO: 4) |
| Mature | APPPNLPDPKFESKAALLAARGPEELLCFTERLEDLVCFW |
| EpoR | EEAASAGVGPGNYSFSYQLEDEPWKLCRIHQAPTARGAVR |
| (human) | FWCSLPTADTSSFVPLELRVTAASGAPRYHRVIHINEVVL |
| | LDAPVGLVARLADESGHVVLRWLPPPETPMTSHIRYEVDV |
| | SAGNGAGSVQRVEILEGRTECVLSNLRGRTRYTFAVRARM |
| | AEPSFGGFWSAWSEPVSLLTPSDLDPLILTLSLILVVILV |
| | LLTVLALLSHRRALKQKIWPGIPSPESEFEGLFTTHKGNF |
| | QLWLYQNDGCLWWSPCTPFTEDPPASLEVLSERCWGTMQA |
| | VEPGTDDEGPLLEPVGSEHAQDTYLVLDKWLLPRNPPSED |
| | LPGPGGSVDIVAMDEGSEASSCSSALASKPSPEGASAASF |
| | EYTILDPSSQLLRPWTLCPELPPTPPHLKYLYLVVSDSGI |
| | STDYSSGDSQGAQGGLSDGPYSNPYENSLIPAAEPLPPSY |
| | VACS |
| | (SEQ ID NO: 5) |
| Isoform | MDHLGASLWPQVGSLCLLLAGAAWAPPPNLPDPKFESKAA |
| EpoR-S; | LLAARGPEELLCFTERLEDLVCFWEEAASAGVGPGNYSFS |
| soluble | YQLEDEPWKLCRLHQAPTARGAVRFWCSLPTADTSSFVPL |
| form | ELRVTAASGAPRYHRVIHINEVVLLDAPVGLVARLADESG |
| (human) | HVVLRWLPPPETPMTSHIRYEVDVSAGNGAGSVQRGIVFL |
| | SPDWLSSTRARPHVIYFCLLRVPRPDSAPRWRSWRAAPSV |
| | C |
| | (SEQ ID NO: 6) |
| Isoform | MDHLGASLWPQVGSLCLLLAGAAWAPPPNLPDPKFESKAA |
| EpoR-T; | LLAARGPEELLCFTERLEDLVCFWEEAASAGVGPGNYSFS |
| truncated | YQLEDEPWKLCRLHQAPTARGAVRFWCSLPTADTSSFVPL |
| form | ELRVTAASGAPRYHRVIHINEVVLLDAPVGLVARLADESG |
| (human) | HVVLRWLPPPETPMTSHIRYEVDVSAGNGAGSVQRVEILE |
| | GRTECVLSNLRGRTRYTFAVRARMAEPSFGGFWSAWSEPV |
| | SLLTPSDLDPLILTLSLILVVILVLLTVLALLSHRRALKQ |
| | KIWPGIPSPESEFEGLFTTHKGNFQVGGLVVPSVPGLPCF |
| | LQPNCRPL |
| | (SEQ ID NO: 7) |

The term “chimeric ligand receptor” as used herein refers to a ligand receptor that comprises domains derived from multiple distinct protein sequences. Chimeric ligand receptors of the present disclosure do not include natural receptors or wild-type receptors, such as a wild-type cytokine receptor or a wild-type erythropoietin receptor (EpoR). A chimeric ligand receptor of the present disclosure comprises one or more receptor subunits. Each of the receptor subunits comprises a scaffold domain, a ligand binding domain, and an intracellular signaling domain.

The term “scaffold domain” as used herein refers to at least a portion of an extracellular domain and transmembrane domain of a receptor that is activated upon ligand binding. The binding of ligand can lead to conformational change(s) or conformation reorganization of the scaffold domain, which modulates activity of an intracellular signaling domain that is operably linked to the scaffold domain. The scaffold domain can be derived from the extracellular domain and transmembrane domain of a cytokine receptor, such as EpoR. In some embodiments, the scaffold domain may be inert to binding of its native ligand. For example, if the scaffold domain is derived from the extracellular domain and transmembrane domain of EpoR, it is inert to erythropoietin binding.

The term “ligand binding domain” as used herein refers to the domain of a chimeric ligand binding receptor of the present disclosure that is operably linked to the extracellular domain of the scaffold domain. Ligand binding domains of the present disclosure are chimeric in that they are not derived from the same parental protein as the scaffold domain and do not bind to the native ligand of the receptor that the scaffold domain is derived from. For example, if the scaffold domain is derived from EpoR, the ligand binding domain does not bind to the native ligand of EpoR, erythropoietin.

The term “intracellular signaling domain” as used herein refers to the domain of the chimeric ligand binding receptor that is operably linked to the transmembrane domain of the scaffold domain. Intracellular signaling domains of the present disclosure are chimeric in that they are not derived from the same parental protein as the scaffold domain and are not activated by the native ligand of the parental receptor that the scaffold domain is derived from. For example, if the scaffold domain is derived from EpoR, the intracellular signaling domain is not activated by the native ligand of EpoR, erythropoietin.

The term “native ligand” as used herein refers to a wild-type ligand that naturally binds to the parental receptor from which the scaffold domain is derived. For example, if the scaffold domain is derived from EpoR, the native ligand is erythropoietin. The scaffold domain is inert (i.e., unresponsive) to its native ligand. The scaffold domain can contain modifications that render it unresponsive to its native ligand.

### DETAILED DESCRIPTION

### Chimeric Ligand Receptor

In some aspects, disclosed herein is a chimeric ligand receptor.

In some embodiments, the chimeric ligand receptor comprises one receptor subunit. In some embodiments, the chimeric ligand receptor comprises more than one receptor subunit, such as two receptor subunits, three receptor subunits, four receptor subunits, five receptor subunits, six receptor subunits, or more than six receptor subunits.

In some embodiments, the chimeric ligand receptor subunit comprises a scaffold domain, a ligand binding domain, and an intracellular signaling domain.

In some embodiments, the two or more receptor subunits multimerize via the scaffold domain. In some embodiments, the multimerized receptor subunits comprise a dimer, a trimer, tetramer, pentamer, or hexamer. In some embodiments, the multimerized receptor subunits comprise a dimer.

In some embodiments, the multimerization of the receptor subunits occurs prior to ligand binding. In some embodiments, the multimerized receptor subunits are locked by transmembrane helix interactions in a conformation that prevents downstream signaling in the absence of ligand binding. In some embodiments, the ligand binding leads to a conformational reorganization. In some embodiments, the conformational reorganization may comprise a rotation of each scaffold domain around its own axis. In some embodiments, the conformational reorganization activates the intracellular signaling domains of each receptor subunit. In some embodiments, the conformational reorganization inhibits the intracellular signaling domains of each receptor subunit.

### Scaffold Domain

In some embodiments, the scaffold domain comprises an extracellular domain and a transmembrane domain. In some embodiments, the extracellular domain is operably linked to a ligand binding domain and the transmembrane domain is operably linked to an intracellular signaling domain.

In some embodiments, the scaffold domain comprises the extracellular domain and transmembrane domain of a receptor. In some embodiments, the scaffold domain comprises the extracellular domain and transmembrane domain of a transmembrane receptor, such as a cytokine receptor, having a preformed, inactive, dimeric structure on the cell surface that is activated upon ligand binding by a conformational reorganization (e.g., rotation) of the transmembrane domain (Wee, e.g., Maruyama I N, Bioessays, 2015, 37:959-967). In some embodiments, the scaffold domain comprises the extracellular domain and transmembrane domain of an erythropoietin receptor (EpoR).

In some embodiments, the scaffold domain is inert to its native ligand. In some embodiments, when the scaffold domain comprises the extracellular domain and transmembrane domain of an erythropoietin receptor (EpoR), the scaffold domain is inert to erythropoietin.

In some embodiments, the scaffold domain comprises one or more modifications. In some embodiments, the extracellular domain of the scaffold domain comprises one or more modifications. In some embodiments, the transmembrane domain of the scaffold domain comprises one or more modifications. In some embodiments, both the extracellular domain and the transmembrane domain of the scaffold domain comprise one or more modifications. In some embodiments, the modification comprises an amino acid insertion, an amino acid deletion, or an amino acid substitution. In some embodiments, the modification comprises a chemical modification, such as but not limited to acetylation, amidation, biotinylation, cysteinylation, deamidation, farnesylation, formylation, geranylgeranylation, glutathionylation, glycation, glycosylation, hydroxylation, methylation, mono-ADP-ribosylation, myristoylation, oxidation, palmitoylation, phosphorylation, poly(ADP-ribosyl)ation, stearoylation, or sulfation. In some embodiments, the modification renders the scaffold domain inert to its native ligand. In some embodiments, when the scaffold domain comprises the extracellular domain and transmembrane domain of an erythropoietin receptor (EpoR), the extracellular domain comprises an F93A amino acid substitution. In some embodiments, one or more additional amino acid residues are inserted adjacent to the transmembrane domain. In some embodiments, one or more additional amino acid residues are inserted within the transmembrane domain. In some embodiments, the one or more additional amino acid residues are alanine residues. In some embodiment, one or more positively charged amino acid residues are inserted C-terminal to the transmembrane domain. In some embodiments, the transmembrane domain further comprises one, two, three, or four additional alanine residues. In some embodiments, the one or more additional amino acid residues are inserted C-terminal to the transmembrane domain.

In some embodiments, the scaffold domain comprises an extracellular domain and transmembrane domain comprising an amino acid sequence having 60-100% sequence identity to SEQ ID NO: 8, such as 70-100%, 80-100%, 85-100%, 90-100%, 95-100%, 97-100%, or 99-100% sequence identity to APSPSLPDPKFESKAALLASRGSEELLCFTQRLEDLVCFWEEAASSGMDFNYSFSYQLEG ESRKSCSLHQAPTVRGSVRFWCSLPTADTSSFVPLELQVTEASGSPRYHRIIHINEVVLLD APAGLLARRAEEGSHVVLRWLPPPGAMTTHIRYEVDVSAGNRAGGTQRVEVLEGRTE CVLSNLRGGTRYTFAVRARMAEPSFSGFWSAWSEPASLLTASDLDPLILTLSLILVLISLL LTVLALLS (SEQ ID NO: 8). In some embodiments, the scaffold domain comprises an extracellular domain and transmembrane domain comprising an amino acid sequence having 60% or greater sequence identity to SEQ ID NO: 8. In some embodiments, the scaffold domain comprises an extracellular domain and transmembrane domain comprising an amino acid sequence having 70% or greater sequence identity to SEQ ID NO: 8. In some embodiments, the scaffold domain comprises an extracellular domain and transmembrane domain comprising an amino acid sequence having 80% or greater sequence identity to SEQ ID NO: 8. In some embodiments, the scaffold domain comprises an extracellular domain and transmembrane domain comprising an amino acid sequence having 85% or greater sequence identity to SEQ ID NO: 8. In some embodiments, the scaffold domain comprises an extracellular domain and transmembrane domain comprising an amino acid sequence having 90% or greater sequence identity to SEQ ID NO: 8. In some embodiments, the scaffold domain comprises an extracellular domain and transmembrane domain comprising an amino acid sequence having 95% or greater sequence identity to SEQ ID NO: 8. In some embodiments, the scaffold domain comprises an extracellular domain and transmembrane domain comprising an amino acid sequence having 97% or greater sequence identity to SEQ ID NO: 8. In some embodiments, the scaffold domain comprises an extracellular domain and transmembrane domain comprising an amino acid sequence having 99% or greater sequence identity to SEQ ID NO: 8. In some embodiments, the scaffold domain comprises an extracellular domain and transmembrane domain comprising an amino acid sequence having 85%, 86%, 87%, 88%, 89%, 90%, 91%, 92%, 93%, 94%, 95%, 96%, 97%, 98%, 99%, or 100% sequence identity to SEQ ID NO: 8. In some embodiments, the scaffold domain comprises an extracellular domain and transmembrane domain comprising an amino acid sequence having 100% sequence identity to SEQ ID NO: 8.

In some embodiments, the extracellular domain comprises an amino acid sequence having 60-100% sequence identity to SEQ ID NO: 9, such as 70-100%, 80-100%, 85-100%, 90-100%, 95-100%, 97-100%, or 99-100% sequence identity to APSPSLPDPKFESKAALLASRGSEELLCFTQRLEDLVCFWEEAASSGMDFNYSFSYQLEG ESRKSCSLHQAPTVRGSVRFWCSLPTADTSSFVPLELQVTEASGSPRYHRIIHINEVVLLD APAGLLARRAEEGSHVVLRWLPPPGAPMTTHIRYEVDVSAGNRAGGTQRVEVLEGRTE CVLSNLRGGTRYTFAVRARMAEPSFSGFWSAWSEPASLLTASDLDP (SEQ ID NO: 9). In some embodiments, the extracellular domain comprises an amino acid sequence having 60% or greater sequence identity to SEQ ID NO: 9. In some embodiments, the extracellular domain comprises an amino acid sequence having 70% or greater sequence identity to SEQ ID NO: 9. In some embodiments, the extracellular domain comprises an amino acid sequence having 80% or greater sequence identity to SEQ ID NO: 9. In some embodiments, the extracellular domain comprises an amino acid sequence having 85% or greater sequence identity to SEQ ID NO: 9. In some embodiments, the extracellular domain comprises an amino acid sequence having 90% or greater sequence identity to SEQ ID NO: 9. In some embodiments, the extracellular domain comprises an amino acid sequence having 95% or greater sequence identity to SEQ ID NO: 9. In some embodiments, the extracellular domain comprises an amino acid sequence having 97% or greater sequence identity to SEQ ID NO: 9. In some embodiments, the extracellular domain comprises an amino acid sequence having 99% or greater sequence identity to SEQ ID NO: 9. In some embodiments, the extracellular domain comprises an amino acid sequence having 85%, 86%, 87%, 88%, 89%, 90%, 91%, 92%, 93%, 94%, 95%, 96%, 97%, 98%, 99%, or 100% sequence identity to SEQ ID NO: 9. In some embodiments, the extracellular domain comprises an amino acid sequence having 100% sequence identity to SEQ ID NO: 9.

In some embodiments, the transmembrane domain comprises an amino acid sequence having 60-100% sequence identity to SEQ ID NO: 10, such as 70-100%, 80-100%, 85-100%, 90-100%, 95-100%, 97-100%, or 99-100% sequence identity to LILTLSLILVLISLLLTVLALLS (SEQ ID NO: 10). In some embodiments, the transmembrane domain comprises an amino acid sequence having 60% or greater sequence identity to SEQ ID NO: 10. In some embodiments, the transmembrane domain comprises an amino acid sequence having 70% or greater sequence identity to SEQ ID NO: 10. In some embodiments, the transmembrane domain comprises an amino acid sequence having 80% or greater sequence identity to SEQ ID NO: 10. In some embodiments, the transmembrane domain comprises an amino acid sequence having 85% or greater sequence identity to SEQ ID NO: 10. In some embodiments, the transmembrane domain comprises an amino acid sequence having 90% or greater sequence identity to SEQ ID NO: 10. In some embodiments, the transmembrane domain comprises an amino acid sequence having 95% or greater sequence identity to SEQ ID NO: 10. In some embodiments, the transmembrane domain comprises an amino acid sequence having 97% or greater sequence identity to SEQ ID NO: 10. In some embodiments, the transmembrane domain comprises an amino acid sequence having 99% or greater sequence identity to SEQ ID NO: 10. In some embodiments, the transmembrane domain comprises an amino acid sequence having 85%, 86%, 87%, 88%, 89%, 90%, 91%, 92%, 93%, 94%, 95%, 96%, 97%, 98%, 99%, or 100% sequence identity to SEQ ID NO: 10. In some embodiments, the transmembrane domain comprises an amino acid sequence having 100% sequence identity to SEQ ID NO: 10.

In some embodiments, the scaffold domain comprises an extracellular domain comprising an amino acid sequence having 80-100% sequence identity to SEQ ID NO: 9, such as 85-100%, 90-100%, 95-100%, 97-100%, or 99-100% sequence identity to SEQ ID NO: 9 and a transmembrane domain comprising an amino acid sequence having 80-100% sequence identity to SEQ ID NO: 10 such as 85-100%, 90-100%, 95-100%, 97-100%, or 99-100% sequence identity to SEQ ID NO: 10. In some embodiments, the scaffold domain comprises an extracellular domain comprising an amino acid sequence having 80% or greater sequence identity to SEQ ID NO: 9 and a transmembrane domain comprising an amino acid sequence having 80% or greater sequence identity to SEQ ID NO: 10. In some embodiments, the scaffold domain comprises an extracellular domain comprising an amino acid sequence having 85% or greater sequence identity to SEQ ID NO: 9 and a transmembrane domain comprising an amino acid sequence having 85% or greater sequence identity to SEQ ID NO: 10. In some embodiments, the scaffold domain comprises an extracellular domain comprising an amino acid sequence having 90% or greater sequence identity to SEQ ID NO: 9 and a transmembrane domain comprising an amino acid sequence having 90% or greater sequence identity to SEQ ID NO: 10. In some embodiments, the scaffold domain comprises an extracellular domain comprising an amino acid sequence having 95% or greater sequence identity to SEQ ID NO: 9 and a transmembrane domain comprising an amino acid sequence having 95% or greater sequence identity to SEQ ID NO: 10. In some embodiments, the scaffold domain comprises an extracellular domain comprising an amino acid sequence having 97% or greater sequence identity to SEQ ID NO: 9 and a transmembrane domain comprising an amino acid sequence having 97% or greater sequence identity to SEQ ID NO: 10. In some embodiments, the scaffold domain comprises an extracellular domain comprising an amino acid sequence having 99% or greater sequence identity to SEQ ID NO: 9 and a transmembrane domain comprising an amino acid sequence having 99% or greater sequence identity to SEQ ID NO: 10. In some embodiments, the scaffold domain comprises an extracellular domain comprising an amino acid sequence having 85%, 86%, 87%, 88%, 89%, 90%, 91%, 92%, 93%, 94%, 95%, 96%, 97%, 98%, 99%, or 100% sequence identity to SEQ ID NO: 9 and a transmembrane domain comprising an amino acid sequence having 85%, 86%, 87%, 88%, 89%, 90%, 91%, 92%, 93%, 94%, 95%, 96%, 97%, 98%, 99%, or 100% sequence identity to SEQ ID NO: 10. In some embodiments, the scaffold domain comprises an extracellular domain comprising an amino acid sequence having 100% sequence identity to SEQ ID NO: 9 and a transmembrane domain comprising an amino acid sequence having 100% sequence identity to SEQ ID NO: 10.

In some embodiments, the scaffold domain is derived from a leptin receptor (Ob-R) (See, e.g., Couturier C and Jockers R, J Biol Chem, 2003, 278:26604-26611; Carpenter B et al., Structure, 2012, 20:487-497). In some embodiments, the Ob-R comprises the amino acid sequence of

(SEQ ID NO: 11)
MICQKFCVVLLHWEFIYVITAFNLSYPITPWRFKLSCMPPNSTYDYFLL
PAGLSKNTSNSNGHYETAVEPKFNSSGTHFSNLSKTTFHCCFRSEQDRN
CSLCADNIEGKTFVSTVNSLVFQQIDANWNIQCWLKGDLKLFICYVESL
FKNLFRNYNYKVHLLYVLPEVLEDSPLVPQKGSFQMVHCNCSVHECCEC
LVPVPTAKLNDTLLMCLKITSGGVIFQSPLMSVQPINMVKPDPPLGLHM
EITDDGNLKISWSSPPLVPFPLQYQVKYSENSTTVIREADKIVSATSLL
VDSILPGSSYEVQVRGKRLDGPGIWSDWSTPRVFTTQDVIYFPPKILTS
VGSNVSFHCIYKKENKIVPSKEIVWWMNLAEKIPQSQYDVVSDHVSKVT
FFNLNETKPRGKFTYDAVYCCNEHECHHRYAELYVIDVNINISCETDGY
LTKMTCRWSTSTIQSLAESTLQLRYGHRSSLYCSDIPSIHPISEPKDCY
LQSDGFYECIFQPIFLLSGYTMWIRINHSLGSLDSPPTCVLPDSVVKPL
PPSSVKAEITINIGLLKISWEKPVFPENNLQFQIRYGLSGKEVQWKMYE
VYDAKSKSVSLPVPDLCAVYAVQVRCKRLDGLGYWSNWSNPAYTVVMDI
KVPMRGPEFWRIINGDTMKKEKNVTLLWKPLMKNDSLCSVQRYVINHHT
SCNGTWSEDVGNHTKFTFLWTEQAHTVTVLAINSIGASVANFNLTFSWP
MSKVNIVQSLSAYPLNSSCVIVSWILSPSDYKLMYFIIEWKNLNEDGEI
KWLRISSSVKKYYIHDHFIPIEKYQFSLYPIFMEGVGKPKIINSFTQDD
IEKHQSDAGLYVIVPVIISSSILLLGTLLISHQRMKKLFWEDVPNPKNC
SWAQGLNFQKPETFEHLFIKHTASVTCGPLLLEPETISEDISVDTSWKN
KDEMMPTTVVSLLSTTDLEKGSVCISDQFNSVNFSEAEGTEVTYEDESQ
RQPFVKYATLISNSKPSETGEEQGLINSSVTKCFSSKNSPLKDSFSNSS
WEIEAQAFFILSDQHPNIISPHLTFSEGLDELLKLEGNFPEENNDKKSI
YYLGVTSIKKRESGVLLTDKSRVSCPFPAPCLFTDIRVLQDSCSHFVEN
NINLGTSSKKTFASYMPQFQTCSTQTHKIMENKMCDLTV.

In some embodiments, the scaffold domain comprises an extracellular domain and transmembrane domain comprising an amino acid sequence having 60-100% sequence identity to SEQ ID NO: 12, such as 70-100%, 80-100%, 85-100%, 90-100%, 95-100%, 97-100%, or 99-100% sequence identity to FNLSYPITPWRFKLSCMPPNSTYDYFLLPAGLSKNTSNSNGHYETAVEPKFNSSGTHFSN LSKTTFHCCFRSEQDRNCSLCADNIEGKTFVSTVNSLVFQQIDANWNIQCWLKGDLKLFI CYVESLFKNLFRNYNYKVHLLYVLPEVLEDSPLVPQKGSFQMVHCNCSVHECCECLVP VPTAKLNDTLLMCLKITSGGVIFQSPLMSVQPINMVKPDPPLGLHMEITDDGNLKISWSS PPLVPFPLQYQVKYSENSTTVIREADKIVSATSLLVDSILPGSSYEVQVRGKRLDGPGIWS DWSTPRVFTTQDVIYFPPKILTSVGSNVSFHCIYKKENKIVPSKEIVWWMNLAEKIPQSQ YDVVSDHVSKVTFFNLNETKPRGKFTYDAVYCCNEHECHHRYAELYVIDVNINISCETD GYLTKMTCRWSTSTIQSLAESTLQLRYHRSSLYCSDIPSIHPISEPKDCYLQSDGFYECIFQ PIFLLSGYTMWIRINHSLGSLDSTPPTCVLPDSVVKPLPPSSVKAEITINIGLLKISWEKPVFP ENNLQFQIRYGLSGKEVQWKMYEVYDAKSKSVSLPVPDLCAVYAVQVRCKRLDGLGY WSNWSNPAYTVVMDIKVPMRGPEFWRIINGDTMKKEKNVTLLWKPLMKNDSLCSVQR YVINTHHTSCNGTWSEDVGNHTKFTFLWTEQAHTVTVLAINSIGASVANFNLTFSWPMSK VNIVQSLSAYPLNSSCVIVSWILSPSDYKLMYFIIEWKNLNEDGEIKWLRISSSVKKYYIH DHFIPIEKYQFSLYPIFMEGVGKPKIINSFTQDDIEKHQSDAGLYVIVPVIISSSILLLGTLLI (SEQ NO: 12). In some embodiments, the scaffold domain comprises an extracellular domain and transmembrane domain comprising an amino acid sequence having 60% or greater sequence identity to SEQ ID NO: 12. In some embodiments, the scaffold domain comprises an extracellular domain and transmembrane domain comprising an amino acid sequence having 70% or greater sequence identity to SEQ ID NO: 12. In some embodiments, the scaffold domain comprises an extracellular domain and transmembrane domain comprising an amino acid sequence having 80% or greater sequence identity to SEQ ID NO: 12. In some embodiments, the scaffold domain comprises an extracellular domain and transmembrane domain comprising an amino acid sequence having 85% or greater sequence identity to SEQ ID NO: 12. In some embodiments, the scaffold domain comprises an extracellular domain and transmembrane domain comprising an amino acid sequence having 90% or greater sequence identity to SEQ ID NO: 12. In some embodiments, the scaffold domain comprises an extracellular domain and transmembrane domain comprising an amino acid sequence having 95% or greater sequence identity to SEQ ID NO: 12. In some embodiments, the scaffold domain comprises an extracellular domain and transmembrane domain comprising an amino acid sequence having 97% or greater sequence identity to SEQ ID NO: 12. In some embodiments, the scaffold domain comprises an extracellular domain and transmembrane domain comprising an amino acid sequence having 99% or greater sequence identity to SEQ ID NO: 12. In some embodiments, the scaffold domain comprises an extracellular domain and transmembrane domain comprising an amino acid sequence having 85%, 86%, 87%, 88%, 89%, 90%, 91%, 92%, 93%, 94%, 95%, 96%, 97%, 98%, 99%, or 100% sequence identity to SEQ ID NO: 12. In some embodiments, the scaffold domain comprises an extracellular domain and transmembrane domain comprising an amino acid sequence having 100% sequence identity to SEQ ID NO: 12.

In some embodiments, the extracellular domain comprises an amino acid sequence having 60-100% sequence identity to SEQ ID NO: 13, such as 70-100%, 80-100%, 85-100%, 90-100%, 95-100%, 97-100%, or 99-100% sequence identity to FNLSYPITPWRFKLSCMPPNSTYDYFLLPAGLSKNTSNSNGHYETAVEPKFNSSGTHFSN LSKTTFHCCFRSEQDRNCSLCADNIEGKTFVSTVNSLVFQQIDANWNIQCWLKGDLKLFI CYVESLFKNLFRNYNYKVHLLYVLPEVLEDSPLVPQKGSFQMVHCNSVHECCECLVP VPTAKLNDTLLMCLKITSGGVIFQSPLMSVQPINMVKPDPPLGLHMEITDDGNLKISWSS PPLVPFPLQYQVKYSENSTTVIREADKIVSATSLLVDSILPGSSYEVQVRGKRLDGPGIWS DWSTPRVFTTQDVIYFPPKILTSVGSNVSFHCIYKKENKIVPSKEIVWWMNLAEKIPQSQ YDVVSDHVSKVTFFNLNETKPRGKFTYDAVYCCNEHECHHRYAELYVIDVNINISCETD GYLTKMTCRWSTSTIQSLAESTLQLRYHRSSLYCSDIPSIHPISEPKDCYLQSDGFYECIFQ PIFLLSGYTMWIRINHSLGSLDSPPTCVLPDSVVKPLPPSSVKAEITINIGLLKISWEKPVFP ENNLQFQIRYGLSGKEVQWKMYEVYDAKSKSVSLPVPDLCAVYAVQVRCKRLDGLGY WSNWSNPAYTVVMDIKVPMRGPEFWRIINGDTMKKEKNVTLLWKPLMKNDSLCSVQR YVINHHTSCNGTWSEDVGNHTKFTFLWTEQAHTVTVLAINSIGASVANFNLTFSWPMSK VNIVQSLSAYPLNSSCVIVSWILSPSDYKLMYFIIEWKLNEDGEIKWRISSSVKKYYIH DHFIPIEKYQFSLYPIFMEGVGKPKIINSFTQDDIEKHQSD (SEQ ID NO: 13). In some embodiments, the extracellular domain comprises an amino acid sequence having 60% or greater sequence identity to SEQ ID NO: 13. In some embodiments, the extracellular domain comprises an amino acid sequence having 70% or greater sequence identity to SEQ ID NO: 13. In some embodiments, the extracellular domain comprises an amino acid sequence having 80% or greater sequence identity to SEQ ID NO: 13. In some embodiments, the extracellular domain comprises an amino acid sequence having 85% or greater sequence identity to SEQ ID NO: 13: In some embodiments, the extracellular domain comprises an amino acid sequence having 90% or greater sequence identity to SEQ ID NO: 13. In some embodiments, the extracellular domain comprises an amino acid sequence having 95% or greater sequence identity to SEQ ID NO: 13. In some embodiments, the extracellular domain comprises an amino acid sequence having 97% or greater sequence identity to SEQ ID NO: 13. In some embodiments, the extracellular domain comprises an amino acid sequence having 99% or greater sequence identity to SEQ ID NO: 13. In some embodiments, the extracellular domain comprises an amino acid sequence having 85%, 86%, 87%, 88%, 89%, 90%, 91%, 92%, 93%, 94%, 95%, 96%, 97%, 98%, 99%, or 100% sequence identity to SEQ ID NO: 13. In some embodiments, the extracellular domain comprises an amino acid sequence having 100% sequence identity to SEQ ID NO: 13.

In some embodiments, the transmembrane domain comprises an amino acid sequence having 60-100% sequence identity to SEQ ID NO: 14, such as 70-100%, 80-100%, 85-100%, 90-100%, 95-100%, 97-100%, or 99-100% sequence identity to AGLYVIVPVIISSSILLLGTLLI (SEQ ID NO: 14). In some embodiments, the transmembrane domain comprises an amino acid sequence having 60% or greater sequence identity to SEQ ID NO: 14. In some embodiments, the transmembrane domain comprises an amino acid sequence having 70% or greater sequence identity to SEQ ID NO: 14. In some embodiments, the transmembrane domain comprises an amino acid sequence having 80% or greater sequence identity to SEQ ID NO: 14. In some embodiments, the transmembrane domain comprises an amino acid sequence having 85% or greater sequence identity to SEQ ID NO: 14. In some embodiments, the transmembrane domain comprises an amino acid sequence having 90% or greater sequence identity to SEQ ID NO: 14. In some embodiments, the transmembrane domain comprises an amino acid sequence having 95% or greater sequence identity to SEQ ID NO: 14. In some embodiments, the transmembrane domain comprises an amino acid sequence having 97% or greater sequence identity to SEQ ID NO: 14. In some embodiments, the transmembrane domain comprises an amino acid sequence having 99% or greater sequence identity to SEQ ID NO: 14. In some embodiments, the transmembrane domain comprises an amino acid sequence having 85%, 86%, 87%, 88%, 89%, 90%, 91%, 92%, 93%, 94%, 95%, 96%, 97%, 98%, 99%, or 100% sequence identity to SEQ ID NO: 14. In some embodiments, the transmembrane domain comprises an amino acid sequence having 100% sequence identity to SEQ ID NO: 14.

In some embodiments, the scaffold domain comprises an extracellular domain comprising an amino acid sequence having 80-100% sequence identity to SEQ ID NO: 13, such as 85-100%, 90-100%, 95-100%, 97-100%, or 99-100% sequence identity to SEQ ID NO: 13 and a transmembrane domain comprising an amino acid sequence having 80-100% sequence identity to SEQ ID NO: 14, such as 85-100%, 90-100%, 95-100%, 97-100%, or 99-100% sequence identity to SEQ ID NO: 14. In some embodiments, the scaffold domain comprises an extracellular domain comprising an amino acid sequence having 80% or greater sequence identity to SEQ ID NO: 13 and a transmembrane domain comprising an amino acid sequence having 80% or greater sequence identity to SEQ ID NO: 14. In some embodiments, the scaffold domain comprises an extracellular domain comprising an amino acid sequence having 85% or greater sequence identity to SEQ ID NO: 13 and a transmembrane domain comprising an amino acid sequence having 85% or greater sequence identity to SEQ ID NO: 14. In some embodiments, the scaffold domain comprises an extracellular domain comprising an amino acid sequence having 90% or greater sequence identity to SEQ ID NO: 13 and a transmembrane domain comprising an amino acid sequence having 90% or greater sequence identity to SEQ NO: 14. In some embodiments, the scaffold domain comprises an extracellular domain comprising an amino acid sequence having 95% or greater sequence identity to SEQ ID NO: 13 and a transmembrane domain comprising an amino acid sequence having 95% or greater sequence identity to SEQ ID NO: 14. In some embodiments, the scaffold domain comprises an extracellular domain comprising an amino acid sequence having 97% or greater sequence identity to SEQ ID NO: 13 and a transmembrane domain comprising an amino acid sequence having 97% or greater sequence identity to SEQ ID NO: 14. In some embodiments, the scaffold domain comprises an extracellular domain comprising an amino acid sequence having 99% or greater sequence identity to SEQ ID NO: 13 and a transmembrane domain comprising an amino acid sequence having 99% or greater sequence identity to SEQ ID NO: 14. In some embodiments, the scaffold domain comprises an extracellular domain comprising an amino acid sequence having 85%, 86%, 87%, 88%, 89%, 90%, 91%, 92%, 93%, 94%, 95%, 96%, 97%, 98%, 99%, or 100% sequence identity to SEQ ID NO: 13 and a transmembrane domain comprising an amino acid sequence having 85%, 86%, 87%, 88%, 89%, 90%, 91%, 92%, 93%, 94%, 95%, 96%, 97%, 98%, 99%, or 100% sequence identity to SEQ ID NO: 14. In some embodiments, the scaffold domain comprises an extracellular domain comprising an amino acid sequence having 100% sequence identity to SEQ ID NO: 13 and a transmembrane domain comprising an amino acid sequence having 100% sequence identity to SEQ ID NO: 14.

In some embodiments, the scaffold is derived from a growth hormone receptor (GHR) (See, e.g., Brown R J et al., Nat Struct Mol Biol, 2005, 12:814-821). In some embodiments, the GHR comprises the amino acid sequence of

(SEQ ID NO: 15)
MDLWQLLLTLALAGSSDAFSGSEATAAILSRAPWSLQSVNPGLKTNSSK
EPKFTKCRSPERETFSCHWTDEVHHGTKNLGPIQLFYTRRNTQEWTQEW
KECPDYVSAGENSCYFNSSFTSIWIPYCIKLTSNGGTVDEKCFSVDEIV
QPDPPIALNWTLLNVSLTGIHADIQVRWEAPRNADIQKGWMVLEYELQY
KEVNETKWKMMDPILTTSVPVYSLKVDKEYEVRVRSKQRNSGNYGEFSE
VLYVTLPQMSQFTCEEDFYFPWLLIIFGIFGLTVMLFVFLFSKQQRIKM
LILPPVPVPKIKGIDPDLLKEGKLEEVNTILAIHDSYKPEFHSDDSWVE
FIELDIDEPDEKTEESDTDRLLSSDHEKSHSNLGVKDGDSGRTSCCEPD
ILETDFNANDIHEGTSEVAQPQRLKGEADLLCLDQKNQNNSPYHDACPA
TQQPSVIQAEKNKPQPLPTEGAESTHQAAHIQLSNPSSLSNIDFYAQVS
DITPAGSVVLSPGQKNKAGMSQCDMHPEMVSLCQENFLMDNAYFCEADA
KKCIPVAPHIKVESHIQPSLNQEDIYITTESLTTAAGRPGTGEHVPGSE
MPVPDYTSIHIVQSPQGLILNATALPLPDKEFLSSCGYVSTDQLNKIMP.

In some embodiments, the scaffold domain comprises an extracellular domain and transmembrane domain comprising an amino acid sequence having 60-100% sequence identity to SEQ ID NO: 16, such as 70-100%, 80-100%, 85-100%, 90-100%, 95-100%, 97-100%, or 99-100% sequence identity to FSGSEATAAILSRAPWSLQSVNPGLKTNSSKEPKFTKCRSPERETFSCHWTDEVHHGTKN LGPIQLFYTRRNTQEWTQEWKECPDYVSAGENSCYFNSSFTSIWIPYCIKLTSNGGTVDE KCFSVDEIVQPDPPIALNWTLLNVSLTGIHADIQVRWEAPRNADIQKGWMVLEYELQYK EVNETKWKMMDPILTTSVPYSLKVDKEYEVRVRSKQRNSGNYGEFSENTLYVTLPQMS QFTCEEDFYFPWLLIIIFGIFGLTVMLFVFLFS (SEQ ID NO: 16). In some embodiments, the scaffold domain comprises an extracellular domain and transmembrane domain comprising an amino acid sequence having 60% or greater sequence identity to SEQ ID NO: 16. In some embodiments, the scaffold domain comprises an extracellular domain and transmembrane domain comprising an amino acid sequence having 70% or greater sequence identity to SEQ ID NO: 16. In some embodiments, the scaffold domain comprises an extracellular domain and transmembrane domain comprising an amino acid sequence having 80% or greater sequence identity to SEQ ID NO: 16. In some embodiments, the scaffold domain comprises an extracellular domain and transmembrane domain comprising an amino acid sequence having 85% or greater sequence identity to SEQ ID NO: 16. In some embodiments, the scaffold domain comprises an extracellular domain and transmembrane domain comprising an amino acid sequence having 90% or greater sequence identity to SEQ ID NO: 16. In some embodiments, the scaffold domain comprises an extracellular domain and transmembrane domain comprising an amino acid sequence having 95% or greater sequence identity to SEQ ID NO: 16. In some embodiments, the scaffold domain comprises an extracellular domain and transmembrane domain comprising an amino acid sequence having 97% or greater sequence identity to SEQ ID NO: 16. In some embodiments, the scaffold domain comprises an extracellular domain and transmembrane domain comprising an amino acid sequence having 99% or greater sequence identity to SEQ ID NO: 16. In some embodiments, the scaffold domain comprises an extracellular domain and transmembrane domain comprising an amino acid sequence having 85%, 86%, 87%, 88%, 89%, 90%, 91%, 92%, 93%, 94%, 95%, 96%, 97%, 98%, 99%, or 100% sequence identity to SEQ ID NO: 16. In some embodiments, the scaffold domain comprises an extracellular domain and transmembrane domain comprising an amino acid sequence having 100% sequence identity to SEQ ID NO: 16.

In some embodiments, the extracellular domain comprises an amino acid sequence having 60-100% sequence identity to SEQ ID NO: 17, such as 70-100%, 80-100%, 85-100%, 90-100%, 95-100%, 97-100%, or 99-100% sequence identity to FSGSEATAAILSRAPWSLQSVNPGLKTNSSKEPKFTKCRSPERETFSCHWTDEVHHGTKN LGPIQLFYTRRNTQEWTQEWKECPDYVSAGENSCYFNSSFTSIWIPYCIKLTSNGGTVDE KCFSVDEIVQPDPPIALNWTLLNVSLTGIHADIQVRWEAPRNADIQKGWMVLEYELQYK EVNETKWKMMDPILTTSVPVYSLKVDKEYEVRVRSKQRNSGNYGEFSEVLYTLPQMS QFTCEEDFY (SEQ ID NO: 17). In some embodiments, the extracellular domain comprises an amino acid sequence having 60% or greater sequence identity to SEQ ID NO: 17. In some embodiments, the extracellular domain comprises an amino acid sequence having 70% or greater sequence identity to SEQ ID NO: 17. In some embodiments, the extracellular domain comprises an amino acid sequence having 80% or greater sequence identity to SEQ ID NO: 17. In some embodiments, the extracellular domain comprises an amino acid sequence having 85% or greater sequence identity to SEQ ID NO: 17. In some embodiments, the extracellular domain comprises an amino acid sequence having 90% or greater sequence identity to SEQ ID NO: 17. In some embodiments, the extracellular domain comprises an amino acid sequence having 95% or greater sequence identity to SEQ ID NO: 17. In some embodiments, the extracellular domain comprises an amino acid sequence having 97% or greater sequence identity to SEQ ID NO: 17. In some embodiments, the extracellular domain comprises an amino acid sequence having 99% or greater sequence identity to SEQ ID NO: 17. In some embodiments, the extracellular domain comprises an amino acid sequence having 85%, 86%, 87%, 88%, 89%, 90%, 91%, 92%, 93%, 94%, 95%, 96%, 97%, 98%, 99%, or 100% sequence identity to SEQ ID NO: 17. In some embodiments, the extracellular domain comprises an amino acid sequence having 100% sequence identity to SEQ ID NO: 17.

In some embodiments, the transmembrane domain comprises an amino acid sequence having 60-100% sequence identity to SEQ ID NO: 18, such as 70-100%, 80-100%, 85-100%, 90-100%, 95-100%, 97-100%, or 99-100% sequence identity to FPWLLIIIFGIFGLTVMLFVFLFS (SEQ ID NO: 18). In some embodiments, the transmembrane domain comprises an amino acid sequence having 60% or greater sequence identity to SEQ ID NO: 18. In some embodiments, the transmembrane domain comprises an amino acid sequence having 70% or greater sequence identity to SEQ ID NO: 18. In some embodiments, the transmembrane domain comprises an amino acid sequence having 80% or greater sequence identity to SEQ ID NO: 18. In some embodiments, the transmembrane domain comprises an amino acid sequence having 85% or greater sequence identity to SEQ ID NO: 18. In some embodiments, the transmembrane domain comprises an amino acid sequence having 90% or greater sequence identity to SEQ ID NO: 18. In some embodiments, the transmembrane domain comprises an amino acid sequence having 95% or greater sequence identity to SEQ ID NO: 18. In some embodiments, the transmembrane domain comprises an amino acid sequence having 97% or greater sequence identity to SEQ ID NO: 18. In some embodiments, the transmembrane domain comprises an amino acid sequence having 99% or greater sequence identity to SEQ ID NO: 18. In some embodiments, the transmembrane domain comprises an amino acid sequence having 85%, 86%, 87%, 88%, 89%, 90%, 91%, 92%, 93%, 94%, 95%, 96%, 97%, 98%, 99%, or 100% sequence identity to SEQ ID NO: 18. In some embodiments, the transmembrane domain comprises an amino acid sequence having 100% sequence identity to SEQ ID NO: 18.

In some embodiments, the scaffold domain comprises an extracellular domain comprising an amino acid sequence having 80-100% sequence identity to SEQ ID NO: 17, such as 85-100%, 90-100%, 95-100%, 97-100%, or 99-100% sequence identity to SEQ ID NO: 17 and a transmembrane domain comprising an amino acid sequence having 80-100% sequence identity to SEQ ID NO: 18, such as 85-100%, 90-100%, 95-100%, 97-100%, or 99-100% sequence identity to SEQ ID NO: 18. In some embodiments, the scaffold domain comprises an extracellular domain comprising an amino acid sequence having 80% or greater sequence identity to SEQ ID NO: 17 and a transmembrane domain comprising an amino acid sequence having 80% or greater sequence identity to SEQ ID NO: 18. In some embodiments, the scaffold domain comprises an extracellular domain comprising an amino acid sequence having 85% or greater sequence identity to SEQ ID NO: 17 and a transmembrane domain comprising an amino acid sequence having 85% or greater sequence identity to SEQ ID NO: 18. In some embodiments, the scaffold domain comprises an extracellular domain comprising an amino acid sequence having 90% or greater sequence identity to SEQ ID NO: 17 and a transmembrane domain comprising an amino acid sequence having 90% or greater sequence identity to SEQ ID NO: 18. In some embodiments, the scaffold domain comprises an extracellular domain comprising an amino acid sequence having 95% or greater sequence identity to SEQ ID NO: 17 and a transmembrane domain comprising an amino acid sequence having 95% or greater sequence identity to SEQ ID NO: 18. In some embodiments, the scaffold domain comprises an extracellular domain comprising an amino acid sequence having 97% or greater sequence identity to SEQ ID NO: 17 and a transmembrane domain comprising an amino acid sequence having 97% or greater sequence identity to SEQ ID NO: 18. In some embodiments, the scaffold domain comprises an extracellular domain comprising an amino acid sequence having 99% or greater sequence identity to SEQ ID NO: 17 and a transmembrane domain comprising an amino acid sequence having 99% or greater sequence identity to SEQ ID NO: 18. In some embodiments, the scaffold domain comprises an extracellular domain comprising an amino acid sequence having 85%, 86%, 87%, 88%, 89%, 90%, 91%, 92%, 93%, 94%, 95%, 96%, 97%, 98%, 99%, or 100% sequence identity to SEQ ID NO: 17 and a transmembrane domain comprising an amino acid sequence having 85%, 86%, 87%, 88%, 89%, 90%, 91%, 92%, 93%, 94%, 95%, 96%, 97%, 98%, 99%, or 100% sequence identity to SEQ ID NO: 18. In some embodiments, the scaffold domain comprises an extracellular domain comprising an amino acid sequence having 100% sequence identity to SEQ ID NO: 17 and a transmembrane domain comprising an amino acid sequence having 100% sequence identity to SEQ ID NO: 18.

In some embodiments, the scaffold domain is derived from an epidermal growth factor receptor (EGFR) (See, e.g., Purba E R et al., Cells, 2017, 6(2). pii: E13). In some embodiments, the EGFR comprises the amino acid sequence of

(SEQ ID NO: 19)
MRPSGTAGAALLALLAALCPASRALEEKKVCQGTSNKLTQLGTFEDHFL
SLQRMFNNCEVVLGNLEITYVQRNYDLSFLKTIQEVAGYVLIALNTVER
IPLENLQHIRGNMYYENSYALAVLSNYDANKTGLKELPMRNLQEILHGA
VRFSNNPALCNVESIQWRDIVSSDFLSNMSMDFQNHLGSCQKCDPSCPN
GSCWGAGEENCQKLTKIICAQQCSGRCRGKSPSDCCHNQCAAGCTGPRE
SDCLVCRKFRDEATCKDTCPPLMLYNPTTYQMDVNPEGKYSFGATCVKK
CPRNYVVTDHGSCVRACGADSYEMEEDGVRKCKKCEGPCRKVCNGIGIG
EFKDSLSINATNIKHFKNCTSISGDLHILPVAFRGDSFTHTPPLDPQEL
DILKTVKEITGFLLIQAWPENRTDLHAFENLEIIRGRTKQHGQFSLAVV
SLNITSLGLRSLKEISDGDVIISGNKNLCYANTINWKKLFGTSGQKTKI
ISNRGENSCKATGQVCHALCSPEGCWGPEPRDCVSCRNVSRGRECVDKC
NLLEGEPREFVENSECIQCHPECLPQAMNITCTGRGPDNCIQCAHYIDG
PHCVKTCPAGVMGENNTLVWKYADAGHVCHLCHPNCTYGCTGPGLEGCP
TNGPKIPSIATGMVGALLLLLVVALGIGLFMRRRHIVRKRTLRRLLQER
ELVEPLTPSGEAPNQALLRILKETEFKKIKVLGSGAFGTVYKGIWIPEG
EKVKIPVAIKELREATSPKANKEILDEAYVMASVDNPHVCRLLGICLTS
TVQLITQLMPFGCLLDYVREHKDNIGSQYLLNWCVQIAKGMNYLEDRRL
VHRDLAARNVLVKTPQHVKITDFGLAKLLGAEEKEYHAEGGKVPIKWMA
LESILHRIYTHQSDVWSYGVTVWELMTFGSKPYDGIPASEISSILEKGE
RLPQPPICTIDVYMIMVKCWMIDADSRPKFRELIIEFSKMARDPQRYLV
IQGDERMHLPSPTDSNFYRALMDEEDMDDVVDADEYLIPQQGFFSSPST
SRTPLLSSLSATSNNSTVACIDRNGLQSCPIKEDSFLQRYSSDPTGALT
EDSIDDTFLPVPEYINQSVPKRPAGSVQNPVYHNQPLNPAPSRDPHYQD
PHSTAVGNPEYLNTVQPTCVNSTFDSPAHWAQKGSHQISLDNPDYQQDF
FPKEAKPNGIFKGSTAENAEYLRVAPQSSEFIGA.

In some embodiments, the scaffold domain comprises an extracellular domain and transmembrane domain comprising an amino acid sequence having 60-100% sequence identity to SEQ ID NO: 20, such as 70-100%, 80-100%, 85-100%, 90-100%, 95-100%, 97-100%, or 99-100% sequence identity to LEEKKVCQGTSNKLTQLGTFEDHFLSLQRMFNNCEVVLGNLEITYVQRNYDLSFLKTIQ EVAGYVLIALNTVERIPLENLQIIRGNMYYENSYALAVLSNYDANKTGLKELPMRNLQEI LHGAVRFSNNPALCNVESIQWRDIVSSDFLSNMSMDFQNHLGSCQKCDPSCPNGSCWG AGEENCQKLTKIICAQQCSGRCRGKSPSDCCHNQCAAGCTGPRESDCLVCRKFRDEATC KDTCPPLMLYNPTTYQMDVNPEGKYSFGATCVKKCPRNYVVTDHGSCVRACGADSYE MEEDGVRKCKKCEGPCRKVCNGIGIGEFKDSLSINATNIKHFKNCTSISGDLHILPVAFRG DSFTHTPPLDPQELDILKTVKEITGFLLIQAWPENRTDLHAFENLEIIRGRTKQHGQFSLA VVSLNITSLGLRSLKEISDGDVIISGNKNLCYANTINWKKLFGTSGQKTKIISNRGENSCK ATGQVCHALCSPEGCWGPEPRDCVSCRNVSRGRECVDKCNLLEGEPREFVENSECIQCH PECLPQAMNITCTGRGPDNCIQCAHYIDGPHCVKTCPAGVMGENNTLVWKYADAGHV CHLCHPNCTYGCTGPGLEGCPTNGPKIPSIATGMVGALLLLLVVALGIGLFM (SEQ ID NO: 20). In some embodiments, the scaffold domain comprises an extracellular domain and transmembrane domain comprising an amino acid sequence having 60% or greater sequence identity to SEQ ID NO: 20. In some embodiments, the scaffold domain comprises an extracellular domain and transmembrane domain comprising an amino acid sequence having 70% or greater sequence identity to SEQ ID NO: 20. In some embodiments, the scaffold domain comprises an extracellular domain and transmembrane domain comprising an amino acid sequence having 80% or greater sequence identity to SEQ ID NO: 20. In some embodiments, the scaffold domain comprises an extracellular domain and transmembrane domain comprising an amino acid sequence having 85% or greater sequence identity to SEQ ID NO: 20. In some embodiments, the scaffold domain comprises an extracellular domain and transmembrane domain comprising an amino acid sequence having 90% or greater sequence identity to SEQ ID NO: 20. In some embodiments, the scaffold domain comprises an extracellular domain and transmembrane domain comprising an amino acid sequence having 95% or greater sequence identity to SEQ ID NO: 20. In some embodiments, the scaffold domain comprises an extracellular domain and transmembrane domain comprising an amino acid sequence having 97% or greater sequence identity to SEQ ID NO: 20. In some embodiments, the scaffold domain comprises an extracellular domain and transmembrane domain comprising an amino acid sequence having 99% or greater sequence identity to SEQ ID NO: 20. In some embodiments, the scaffold domain comprises an extracellular domain and transmembrane domain comprising an amino acid sequence having 85%, 86%, 87%, 88%, 89%, 90%, 91%, 92%, 93%, 94%, 95%, 96%, 97%, 98%, 99%, or 100% sequence identity to SEQ ID NO: 20. In some embodiments, the scaffold domain comprises an extracellular domain and transmembrane domain comprising an amino acid sequence having 100% sequence identity to SEQ ID NO: 20.

In some embodiments, the extracellular domain comprises an amino acid sequence having 60-100% sequence identity to SEQ ID NO: 21, such as 70-100%, 80-100%, 85-100%, 90-100%, 95-100%, 97-100%, or 99-100% sequence identity to LEEKKVCQGTSNKLTQLGTFEDHFLSLQRMFNNCEVVLGNLEITYVQRNYDLSFLKTIQ EVAGYVLIALNTVERIPLENLQIIRGNMYYENSYALAVLSNYDANKTGLKELPMRNLQEI LHGAVRFSNNPALCNVESIQWRDIVSSDFLSNMSMDFQNHLGSCQKCDPSCPNGSCWG AGEENCQKLTKIICAQQCSGRCRGKSPSDCCHNQCAAGCTGPRESDCLVCRKFRDEATC KDTCPPLMLYNPTTYQMDVNPEGKYSFGATCVKKCPRNYVVTDHGSCVRACGADSYE MEEDGVRKCKKCEGPCRKVCNGIGIGEFKDSLSINATNIKHFKNCTSISGDLHILPVAFRG DSFTHTPPLDPQELDILKTVKEITGFILIQAWPENRTDLHAFENLEIIRGRTKQHGQFSLA VVSLNITSLGLRSLKEISDGDVIISGNKNLCYANTINWKKLFGTSGQKTKIISNRGENSCK ATGQVCHALCSPEGCWGPEPRDCVSCRNVSRGRECVDKCNLLEGEPREFVENSECIQCH PECLPQAMNITCTGRGPDNCIQCAHYIDGPHCVKTCPAGVMGENNTLVWKYADAGHV CHLCHPNCTYGCTGPGLEGCPTNGPKIPS (SEQ ID NO: 21). In some embodiments, the extracellular domain comprises an amino acid sequence having 60% or greater sequence identity to SEQ ID NO: 21. In some embodiments, the extracellular domain comprises an amino acid sequence having 70% or greater sequence identity to SEQ ID NO: 21. In some embodiments, the extracellular domain comprises an amino acid sequence having 80% or greater sequence identity to SEQ ID NO: 21. In some embodiments, the extracellular domain comprises an amino acid sequence having 85% or greater sequence identity to SEQ ID NO: 21. In some embodiments, the extracellular domain comprises an amino acid sequence having 90% or greater sequence identity to SEQ ID NO: 21. In some embodiments, the extracellular domain comprises an amino acid sequence having 95% or greater sequence identity to SEQ ID NO: 21. In some embodiments, the extracellular domain comprises an amino acid sequence having 97% or greater sequence identity to SEQ ID NO: 21. In some embodiments, the extracellular domain comprises an amino acid sequence having 99% or greater sequence identity to SEQ ID NO: 21. In some embodiments, the extracellular domain comprises an amino acid sequence having 85%, 86%, 87%, 88%, 89%, 90%, 91%, 92%, 93%, 94%, 95%, 96%, 97%, 98%, 99%, or 100% sequence identity to SEQ ID NO: 21. In some embodiments, the extracellular domain comprises an amino acid sequence having 100% sequence identity to SEQ ID NO: 21.

In some embodiments, the transmembrane domain comprises an amino acid sequence having 60-100% sequence identity to SEQ ID NO: 22, such as 70-100%, 80-100%, 85-100%, 90-100%, 95-100%, 97-100%, or 99-100% sequence identity to IATGMVGALLLLLVVALGIGLFM (SEQ ID NO: 22). In some embodiments, the transmembrane domain comprises an amino acid sequence having 60% or greater sequence identity to SEQ ID NO: 22. In some embodiments, the transmembrane domain comprises an amino acid sequence having 70% or greater sequence identity to SEQ ID NO: 22. In some embodiments, the transmembrane domain comprises an amino acid sequence having 80% or greater sequence identity to SEQ ID NO: 22. In some embodiments, the transmembrane domain comprises an amino acid sequence having 85% or greater sequence identity to SEQ ID NO: 22. In some embodiments, the transmembrane domain comprises an amino acid sequence having 90% or greater sequence identity to SEQ ID NO: 22. In some embodiments, the transmembrane domain comprises an amino acid sequence having 95% or greater sequence identity to SEQ ID NO: 22. In some embodiments, the transmembrane domain comprises an amino acid sequence having 97% or greater sequence identity to SEQ ID NO: 22. In some embodiments, the transmembrane domain comprises an amino acid sequence having 99% or greater sequence identity to SEQ ID NO: 22. In some embodiments, the transmembrane domain comprises an amino acid sequence having 85%, 86%, 87%, 88%, 89%, 90%, 91%, 92%, 93%, 94%, 95%, 96%, 97%, 98%, 99%, or 100% sequence identity to SEQ ID NO: 22. In some embodiments, the transmembrane domain comprises an amino acid sequence having 100% sequence identity to SEQ ID NO: 22.

In some embodiments, the scaffold domain comprises an extracellular domain comprising an amino acid sequence having 80-100% sequence identity to SEQ ID NO: 21, such as 85-100%, 90-100%, 95-100%, 97-100%, or 99-100% sequence identity to SEQ ID NO: 21 and a transmembrane domain comprising an amino acid sequence having 80-100% sequence identity to SEQ ID NO: 22, such as 85-100%, 90-100%, 95-100%, 97-100%, or 99-100% sequence identity to SEQ ID NO: 22. In some embodiments, the scaffold domain comprises an extracellular domain comprising an amino acid sequence having 80% or greater sequence identity to SEQ ID NO: 21 and a transmembrane domain comprising an amino acid sequence having 80% or greater sequence identity to SEQ ID NO: 22. In some embodiments, the scaffold domain comprises an extracellular domain comprising an amino acid sequence having 85% or greater sequence identity to SEQ ID NO: 21 and a transmembrane domain comprising an amino acid sequence having 85% or greater sequence identity to SEQ ID NO: 22. In some embodiments, the scaffold domain comprises an extracellular domain comprising an amino acid sequence having 90% or greater sequence identity to SEQ ID NO: 21 and a transmembrane domain comprising an amino acid sequence having 90% or greater sequence identity to SEQ ID NO: 22. In some embodiments, the scaffold domain comprises an extracellular domain comprising an amino acid sequence having 95% or greater sequence identity to SEQ ID NO: 21 and a transmembrane domain comprising an amino acid sequence having 95% or greater sequence identity to SEQ ID NO: 22. In some embodiments, the scaffold domain comprises an extracellular domain comprising an amino acid sequence having 97% or greater sequence identity to SEQ ID NO: 21 and a transmembrane domain comprising an amino acid sequence having 97% or greater sequence identity to SEQ ID NO: 22. In some embodiments, the scaffold domain comprises an extracellular domain comprising an amino acid sequence having 99% or greater sequence identity to SEQ ID NO: 21 and a transmembrane domain comprising an amino acid sequence having 99% or greater sequence identity to SEQ ID NO: 22. In some embodiments, the scaffold domain comprises an extracellular domain comprising an amino acid sequence having 85%, 86%, 87%, 88%, 89%, 90%, 91%, 92%, 93%, 94%, 95%, 96%, 97%, 98%, 99%, or 100% sequence identity to SEQ ID NO: 21 and a transmembrane domain comprising an amino acid sequence having 85%, 86%, 87%, 88%, 89%, 90%, 91%, 92%, 93%, 94%, 95%, 96%, 97%, 98%, 99%, or 100% sequence identity to SEQ ID NO: 22. In some embodiments, the scaffold domain comprises an extracellular domain comprising an amino acid sequence having 100% sequence identity to SEQ ID NO: 21 and a transmembrane domain comprising an amino acid sequence having 100% sequence identity to SEQ ID NO: 22.

In some embodiments, the scaffold domain is derived from a brain-derived neurotrophic receptor, such as Trk-B or p75^{NTR} (See, e.g., Maruyama I N, Bioessays, 2015, 37:959-967).

In some embodiments, the Trk-B comprises the amino acid sequence of

(SEQ ID NO: 23)
MSSWIRWHGPAMARLWGFCWLVVGFWRAAFACPTSCKCSASRIWCSDPS
PGIVAFPRLEPNSVDPENITEIFIANQKRLEIINEDDVEAYVGLRNLTI
VDSGLKFVAHKAIIKNSNLQHINFTRNKLTSLSRKHFRHLDLSELILVG
NPFTCSCDIMWIKTLQEAKSSPDTQDLYCLNESSKNIPLANLQIPNCGI
PSANLAAPNLTVEEGKSITLSCSVAGDPVPNMYWDVGNLVSKHMNETSH
TQGSLRITNISSDDSGKQISCVAENLVGEDQDSVNLTVHFAPTITFLES
PTSDHHWCIPFTVKGNPKPALQWFYNGAILNESKYICTKIHVTNHTEYH
GCLQLDNPTHMNNGDYTLIAKNEYGKDEKQISAHFMGWPGIDDGANPNY
PDVIYEDYGTAANDIGDTTNRSNEIPSTDVTDKTGREHLSVYAVVVIAS
VVGFCLLVMLFLLKLARHSKFGMKGPASVISNDDDSASPLHHISNGSNT
PSSSEGGPDAVIIGMTKIPVIENPQYFGITNSQLKPDTFVQHIKRHNIV
LKRELGEGAFGKVFLAECYNLCPEQDKILVAVKTLKDASDNARKDFHRE
AELLTNLQHEHIVKFYGVCVEGDPLIMVFEYMKHGDLNKFLRAHGPDAV
LMAEGNPPTELTQSQMLHIAQQIAAGMVYLASQHFVHRDLATRNCLVGE
NLLVKIGDFGMSRDVYSTDYYRVGGHTMLPIRWMPPESIMYRKFTTESD
VWSLGVVLWEIFTYGKQPWYQLSNNEVIECITQGRVLQRPRTCPQEVYE
LMLGCWQREPHMRKNIKGIHTLLQNLAKASPVYLDILG.

In some embodiments, the scaffold domain comprises an extracellular domain and transmembrane domain comprising an amino acid sequence having 60-100% sequence identity to SEQ ID NO: 24, such as 70-100%, 80-100%, 85-100%, 90-100%, 95-100%, 97-100%, or 99-100% sequence identity to CPTSCKCSASRIWCSDPSPGIVAFPRLEPNSVDPENITEIFIANQKRLEIINEDDVEAYVGL RNLTIVDSGLKFVAHKAFLKNSNLQHINFTRNKLTSLSRKHFRHLDLSELILVGNPFTCSC DIMWIKTLQEAKSSPDTQDLYCLNESSKNIPLANLQIPNCGLPSANLAAPNLTVTEEGKSIT LSCSVAGDPVPNMYWDVGNLVSKHMNETSHTQGSLRITNISSDDSGKQISCVAENLVGE DQDSVNLTVHFAPTITFLESPTSDHHWCIPFTVKGNPKPALQWFYNGAILNESKYICTKIH VTNHTEYHGCLQLDNPTHMNGDYTLIAKNEYGKDEKQISAHFMGWPGIDDGANPNY PDVIYEDYGTAANDIGDTTNRSNEIPSTDVTDKTGREHLSVYAVVVIASVVGFCLLVMLF LL (SEQ ID NO: 24). In some embodiments, the scaffold domain comprises an extracellular domain and transmembrane domain comprising an amino acid sequence having 60% or greater sequence identity to SEQ ID NO: 24. In some embodiments, the scaffold domain comprises an extracellular domain and transmembrane domain comprising an amino acid sequence having 70% or greater sequence identity to SEQ ID NO: 24. In some embodiments, the scaffold domain comprises an extracellular domain and transmembrane domain comprising an amino acid sequence having 80% or greater sequence identity to SEQ ID NO: 24. In some embodiments, the scaffold domain comprises an extracellular domain and transmembrane domain comprising an amino acid sequence having 85% or greater sequence identity to SEQ ID NO: 24. In some embodiments, the scaffold domain comprises an extracellular domain and transmembrane domain comprising an amino acid sequence having 90% or greater sequence identity to SEQ ID NO: 24. In some embodiments, the scaffold domain comprises an extracellular domain and transmembrane domain comprising an amino acid sequence having 95% or greater sequence identity to SEQ ID NO: 24. In some embodiments, the scaffold domain comprises an extracellular domain and transmembrane domain comprising an amino acid sequence having 97% or greater sequence identity to SEQ ID NO: 24. In some embodiments, the scaffold domain comprises an extracellular domain and transmembrane domain comprising an amino acid sequence having 99% or greater sequence identity to SEQ ID NO: 24. In some embodiments, the scaffold domain comprises an extracellular domain and transmembrane domain comprising an amino acid sequence having 85%, 86%, 87%, 88%, 89%, 90%, 91%, 92%, 93%, 94%, 95%, 96%, 97%, 98%, 99%, or 100% sequence identity to SEQ ID NO: 24. In some embodiments, the scaffold domain comprises an extracellular domain and transmembrane domain comprising an amino acid sequence having 100% sequence identity to SEQ ID NO: 24.

In some embodiments, the extracellular domain comprises an amino acid sequence having 60-100% sequence identity to SEQ ID NO: 25, such as 70-100%, 80-100%, 85-100%, 90-100%, 95-100%, 97-100%, or 99-100% sequence identity to CPTSCKCSASRIWCSDPSPGIVAFPRLEPNSVDPENITEIFIANQKRLEIINEDDVEAYVGL RNLTIVDSCGKFVAHKAFLKNSNLQHINFTRNKLTSLSRKHFRHLDLSELILVGNPFTCSC DIMWIKTLQEAKSSPDTQDLYCLNESSKNIPLANLQIPNCGLPSANLAAPNLTVEEGKSIT LSCSVAGDPVPNMYWDVGNLVSKHMNETSHTQGSLRITNISSDDSGKQISCVAENLVGE DQDSVNLTVHFAPTITFLESPTSDHHWCIPFTVKGNPKPALQWFYNGAILNESKYICTKIH VTNHTEYHGCLQLDNPTHMNNGDYTLIAKNEYGKDEKQISAHFMGWPGIDDGANPNY PDVIYEDYGTAANDIGDTTNRSNEIPSTDVTDKTGREH (SEQ ID NO: 25). In some embodiments, the extracellular domain comprises an amino acid sequence having 60% or greater sequence identity to SEQ ID NO: 25. In some embodiments, the extracellular domain comprises an amino acid sequence having 70% or greater sequence identity to SEQ ID NO: 25. In some embodiments, the extracellular domain comprises an amino acid sequence having 80% or greater sequence identity to SEQ ID NO: 25. In some embodiments, the extracellular domain comprises an amino acid sequence having 85% or greater sequence identity to SEQ ID NO: 25. In some embodiments, the extracellular domain comprises an amino acid sequence having 90% or greater sequence identity to SEQ ID NO: 25. In some embodiments, the extracellular domain comprises an amino acid sequence having 95% or greater sequence identity to SEQ ID NO: 25. In some embodiments, the extracellular domain comprises an amino acid sequence having 97% or greater sequence identity to SEQ ID NO: 25. In some embodiments, the extracellular domain comprises an amino acid sequence having 99% or greater sequence identity to SEQ ID NO: 25. In some embodiments, the extracellular domain comprises an amino acid sequence having 85%, 86%, 87%, 88%, 89%, 90%, 91%, 92%, 93%, 94%, 95%, 96%, 97%. 98%, 99%, or 100% sequence identity to SEQ ID NO: 25. In some embodiments, the extracellular domain comprises an amino acid sequence having 100% sequence identity to SEQ ID NO: 25.

In some embodiments, the transmembrane domain comprises an amino acid sequence having 60-100% sequence identity to SEQ ID NO: 26, such as 70-100%, 80-100%, 85-100%, 90-100%, 95-100%, 97-100%, or 99-100% sequence identity to LSVYAVVVIASVVGFCLLVMLFLL (SEQ ID NO: 26). In some embodiments, the transmembrane domain comprises an amino acid sequence having 60% or greater sequence identity to SEQ ID NO: 26. In some embodiments, the transmembrane domain comprises an amino acid sequence having 70% or greater sequence identity to SEQ ID NO: 26. In some embodiments, the transmembrane domain comprises an amino acid sequence having 80% or greater sequence identity to SEQ ID NO: 26. In some embodiments, the transmembrane domain comprises an amino acid sequence having 85% or greater sequence identity to SEQ ID NO: 26. In some embodiments, the transmembrane domain comprises an amino acid sequence having 90% or greater sequence identity to SEQ ID NO: 26. In some embodiments, the transmembrane domain comprises an amino acid sequence having 95% or greater sequence identity to SEQ ID NO: 26. In some embodiments, the transmembrane domain comprises an amino acid sequence having 97% or greater sequence identity to SEQ ID NO: 26. In some embodiments, the transmembrane domain comprises an amino acid sequence having 99% or greater sequence identity to SEQ ID NO: 26. In some embodiments, the transmembrane domain comprises an amino acid sequence having 85%, 86%, 87%, 88%, 89%, 90%, 91%, 92%, 93%, 94%, 95%, 96%, 97%, 98%, 99%, or 100% sequence identity to SEQ ID NO: 26. In some embodiments, the transmembrane domain comprises an amino acid sequence having 100% sequence identity to SEQ ID NO: 26.

In some embodiments, the scaffold domain comprises an extracellular domain comprising an amino acid sequence having 80-100% sequence identity to SEQ ID NO: 25, such as 85-100%, 90-100%, 95-100%, 97-100%, or 99-100% sequence identity to SEQ ID NO: 25 and a transmembrane domain comprising an amino acid sequence having 80-100% sequence identity to SEQ ID NO: 26, such as 85-100%, 90-100%, 95-100%, 97-100%, or 99-100% sequence identity to SEQ ID NO: 26. In some embodiments, the scaffold domain comprises an extracellular domain comprising an amino acid sequence having 80% or greater sequence identity to SEQ ID NO: 25 and a transmembrane domain comprising an amino acid sequence having 80% or greater sequence identity to SEQ ID NO: 26. In some embodiments, the scaffold domain comprises an extracellular domain comprising an amino acid sequence having 85% or greater sequence identity to SEQ ID NO: 25 and a transmembrane domain comprising an amino acid sequence having 85% or greater sequence identity to SEQ ID NO: 26. In some embodiments, the scaffold domain comprises an extracellular domain comprising an amino acid sequence having 90% or greater sequence identity to SEQ ID NO: 25 and a transmembrane domain comprising an amino acid sequence having 90% or greater sequence identity to SEQ ID NO: 26. In some embodiments, the scaffold domain comprises an extracellular domain comprising an amino acid sequence having 95% or greater sequence identity to SEQ ID NO: 25 and a transmembrane domain comprising an amino acid sequence having 95% or greater sequence identity to SEQ ID NO: 26. In some embodiments, the scaffold domain comprises an extracellular domain comprising an amino acid sequence having 97% or greater sequence identity to SEQ ID NO: 25 and a transmembrane domain comprising an amino acid sequence having 97% or greater sequence identity to SEQ ID NO: 26. In some embodiments, the scaffold domain comprises an extracellular domain comprising an amino acid sequence having 99% or greater sequence identity to SEQ ID NO: 25 and a transmembrane domain comprising an amino acid sequence having 99% or greater sequence identity to SEQ ID NO: 26. In some embodiments, the scaffold domain comprises an extracellular domain comprising an amino acid sequence having 85%, 86%, 87%, 88%, 89%, 90%, 91%, 92%, 93%, 94%, 95%, 96%, 97%, 98%, 99%, or 100% sequence identity to SEQ ID NO: 25 and a transmembrane domain comprising an amino acid sequence having 85%, 86%, 87%, 88%, 89%, 90%, 91%, 92%, 93%, 94%, 95%, 96%, 97%, 98%, 99%, or 100% sequence identity to SEQ ID NO: 26. In some embodiments, the scaffold domain comprises an extracellular domain comprising an amino acid sequence having 100% sequence identity to SEQ ID NO: 25 and a transmembrane domain comprising an amino acid sequence having 100% sequence identity to SEQ ID NO: 26.

In some embodiments, the p75^{NTR} comprises the amino acid sequence of

(SEQ ID NO: 27)
MGAGATGRAMDGPRLLLLLLLGVSLGGAKEACPTGLYTHSGECCKACNL
GEGVAQPCGANQTVCEPCLDSVTFSDVVSATEPCKPCTECVGLQSMSAP
CVEADDAVCRCAYGYYQDETTGRCEACRVCEAGSGLVFSCQDKQNTVCE
ECPDGTYSDEANHVDPCLPCTVCEDTERQLRECTRWADAECEEIPGRWI
TRSTPPEGSDSTAPSTQEPEAPPEQDLIASTVAGVVTTVMGSSQPVVTR
GTTDNLIPVYCSILAAVVVGLVAYIAFKRWNSCKQNKQGANSRPVNQTP
PPEGEKLHSDSGISVDSQSLHDQQPHTQTASGQALKGDGGLYSSLPPAK
REEVEKLLNGSAGDTWRHLAGELGYQPEHIDSFTHEACPVRALLASWAT
QDSATLDALLAALRRIQRADLVESLCSESTATSPV.

In some embodiments, the scaffold domain comprises an extracellular domain and transmembrane domain comprising an amino acid sequence having 60-100% sequence identity to SEQ ID NO: 28, such as 70-100%, 80-100%, 85-100%, 90-100%, 95-100%, 97-100%, or 99-100% sequence identity to KEACPTGLYTHSGECCKACNLGEGVAQPCGANQTVCEPCLDSVTFSDVVSATEPCKPCT ECVGLQSMSAPCVEADDAVCRCAYGYYQDETTGRCEACRVCEAGSGLVFSCQDKQNT VCEECPDGTYSDEANHVDPCLPCTVCEDTERQLRECTRWADAECEEIPGRWITRSTPPE GSDSTAPSTQEPEAPPEQDLIASTIVAGVVTVMGSSQPVVTRGTTDNLIPVYCSILAAVV VGLVAYIAF (SEQ ID NO: 28). In some embodiments, the scaffold domain comprises an extracellular domain and transmembrane domain comprising an amino acid sequence having 60% or greater sequence identity to SEQ ID NO: 28. In some embodiments, the scaffold domain comprises an extracellular domain and transmembrane domain comprising an amino acid sequence having 70% or greater sequence identity to SEQ ID NO: 28. In some embodiments, the scaffold domain comprises an extracellular domain and transmembrane domain comprising an amino acid sequence having 80% or greater sequence identity to SEQ ID NO: 28. In some embodiments, the scaffold domain comprises an extracellular domain and transmembrane domain comprising an amino acid sequence having 85% or greater sequence identity to SEQ ID NO: 28. In some embodiments, the scaffold domain comprises an extracellular domain and transmembrane domain comprising an amino acid sequence having 90% or greater sequence identity to SEQ ID NO: 28. In some embodiments, the scaffold domain comprises an extracellular domain and transmembrane domain comprising an amino acid sequence having 95% or greater sequence identity to SEQ ID NO: 28. In some embodiments, the scaffold domain comprises an extracellular domain and transmembrane domain comprising an amino acid sequence having 97% or greater sequence identity to SEQ ID NO: 28. In some embodiments, the scaffold domain comprises an extracellular domain and transmembrane domain comprising an amino acid sequence having 99% or greater sequence identity to SEQ ID NO: 28. In some embodiments, the scaffold domain comprises an extracellular domain and transmembrane domain comprising an amino acid sequence having 85%, 86%, 87%, 88%, 89%, 90%, 91%, 92%, 93%, 94%, 95%, 96%, 97%, 98%, 99%, or 100% sequence identity to SEQ ID NO: 28. In some embodiments, the scaffold domain comprises an extracellular domain and transmembrane domain comprising an amino acid sequence having 100% sequence identity to SEQ ID NO: 28.

In some embodiments, the extracellular domain comprises an amino acid sequence having 60-100% sequence identity to SEQ ID NO: 29, such as 70-100%, 80-100%, 85-100%, 90-100%, 95-100%, 97-100%, or 99-100% sequence identity to

(SEQ ID NO: 29)
KEACPTGLYTHSGECCKACNLGEGVAQPCGANQTVCEPCLDSVTFSDVV
SATEPCKPCTECVGLQSMSAPCVEADDAVCRCAYGYYQDETTGRCEACR
VCEAGSGLVFSCQDKQNTVCEECPDGTYSDEANHVDPCLPCTVCEDTER
QLRECTRWADAECEEIPGRWITRSTPPEGSDSTAPSTQEPEAPPEQDLI
ASTVAGVVTTVMGSSQPVVTRGTTDN.

In some embodiments, the extracellular domain comprises an amino acid sequence having 60% or greater sequence identity to SEQ ID NO: 29. In some embodiments, the extracellular domain comprises an amino acid sequence having 70% or greater sequence identity to SEQ ID NO: 29. In some embodiments, the extracellular domain comprises an amino acid sequence having 80% or greater sequence identity to SEQ ID NO: 29. In some embodiments, the extracellular domain comprises an amino acid sequence having 85% or greater sequence identity to SEQ ID NO: 29. In some embodiments, the extracellular domain comprises an amino acid sequence having 90% or greater sequence identity to SEQ ID NO: 29. In some embodiments, the extracellular domain comprises an amino acid sequence having 95% or greater sequence identity to SEQ ID NO: 29. In some embodiments, the extracellular domain comprises an amino acid sequence having 97% or greater sequence identity to SEQ ID NO: 29. In some embodiments, the extracellular domain comprises an amino acid sequence having 99% or greater sequence identity to SEQ ID NO: 29. In some embodiments, the extracellular domain comprises an amino acid sequence having 85%, 86%, 87%, 88%, 89%, 90%, 91%, 92%, 93%, 94%, 95%, 96%, 97%, 98%, 99%, or 100% sequence identity to SEQ ID NO: 29. In some embodiments, the extracellular domain comprises an amino acid sequence having 100% sequence identity to SEQ ID NO: 29.

In some embodiments, the transmembrane domain comprises an amino acid sequence having 60-100% sequence identity to SEQ ID NO: 30, such as 70-100%, 80-100%, 85-100%, 90-100%, 95-100%, 97-100%, or 99-100% sequence identity to LIPVYCSILAAVVVGLVAYIAF (SEQ ID NO: 30). In some embodiments, the transmembrane domain comprises an amino acid sequence having 60% or greater sequence identity to SEQ ID NO: 30. In some embodiments, the transmembrane domain comprises an amino acid sequence having 70% or greater sequence identity to SEQ ID NO: 30. In some embodiments, the transmembrane domain comprises an amino acid sequence having 80% or greater sequence identity to SEQ ID NO: 30. In some embodiments, the transmembrane domain comprises an amino acid sequence having 85% or greater sequence identity to SEQ ID NO: 30. In some embodiments, the transmembrane domain comprises an amino acid sequence having 90% or greater sequence identity to SEQ ID NO: 30. In some embodiments, the transmembrane domain comprises an amino acid sequence having 95% or greater sequence identity to SEQ ID NO: 30. In some embodiments, the transmembrane domain comprises an amino acid sequence having 97% or greater sequence identity to SEQ ID NO: 30. In some embodiments, the transmembrane domain comprises an amino acid sequence having 99% or greater sequence identity to SEQ ID NO: 30. In some embodiments, the transmembrane domain comprises an amino acid sequence having 85%, 86%, 87%, 88%, 89%, 90%, 91%, 92%, 93%, 94%, 95%, 96%, 97%, 98%, 99%, or 100% sequence identity to SEQ ID NO: 30. In some embodiments, the transmembrane domain comprises an amino acid sequence having 100% sequence identity to SEQ ID NO: 30.

In some embodiments, the scaffold domain comprises an extracellular domain comprising an amino acid sequence having 80-100% sequence identity to SEQ ID NO: 29, such as 85-100%, 90-100%, 95-100%, 97-100%, or 99-100% sequence identity to SEQ ID NO: 29 and a transmembrane domain comprising an amino acid sequence having 80-100% sequence identity to SEQ ID NO: 30, such as 85-100%, 90-100%, 95-100%, 97-100%, or 99-100% sequence identity to SEQ ID NO: 30. In some embodiments, the scaffold domain comprises an extracellular domain comprising an amino acid sequence having 80% or greater sequence identity to SEQ ID NO: 29 and a transmembrane domain comprising an amino acid sequence having 80% or greater sequence identity to SEQ ID NO: 30. In some embodiments, the scaffold domain comprises an extracellular domain comprising an amino acid sequence having 85% or greater sequence identity to SEQ ID NO: 29 and a transmembrane domain comprising an amino acid sequence having 85% or greater sequence identity to SEQ ID NO: 30. In some embodiments, the scaffold domain comprises an extracellular domain comprising an amino acid sequence having 90% or greater sequence identity to SEQ ID NO: 29 and a transmembrane domain comprising an amino acid sequence having 90% or greater sequence identity to SEQ ID NO: 30. In some embodiments, the scaffold domain comprises an extracellular domain comprising an amino acid sequence having 95% or greater sequence identity to SEQ ID NO: 29 and a transmembrane domain comprising an amino acid sequence having 95% or greater sequence identity to SEQ ID NO: 30. In some embodiments, the scaffold domain comprises an extracellular domain comprising an amino acid sequence having 97% or greater sequence identity to SEQ ID NO: 29 and a transmembrane domain comprising an amino acid sequence having 97% or greater sequence identity to SEQ ID NO: 30. In some embodiments, the scaffold domain comprises an extracellular domain comprising an amino acid sequence having 99% or greater sequence identity to SEQ ID NO: 29 and a transmembrane domain comprising an amino acid sequence having 99% or greater sequence identity to SEQ ID NO: 30. In some embodiments, the scaffold domain comprises an extracellular domain comprising an amino acid sequence having 85%, 86%, 87%, 88%, 89%, 90%, 91%, 92%, 93%, 94%, 95%, 96%, 97%, 98%, 99%, or 100% sequence identity to SEQ ID NO: 29 and a transmembrane domain comprising an amino acid sequence having 85%, 86%, 87%, 88%, 89%, 90%, 91%, 92%, 93%, 94%, 95%, 96%, 97%, 98%, 99%, or 100% sequence identity to SEQ ID NO: 30. In some embodiments, the scaffold domain comprises an extracellular domain comprising an amino acid sequence having 100% sequence identity to SEQ ID NO: 29 and a transmembrane domain comprising an amino acid sequence having 100% sequence identity to SEQ ID NO: 30.

### Ligand Binding Domain

In some embodiments, the ligand binding domain does not bind a native ligand of the scaffold domain. In some embodiments, the ligand binding domain does not comprise an endogenous ligand binding domain of the scaffold domain. In some embodiments, when the scaffold domain is derived from a cytokine receptor, the ligand binding domain does not bind to the cognate cytokine. In some embodiments, when the scaffold domain is derived from a cytokine receptor, the ligand binding domain is not derived from the same cytokine receptor. In some embodiments, when the scaffold domain is derived from an erythropoietin receptor (EpoR), the ligand binding domain does not bind to erythropoietin. In some embodiments, when the scaffold domain is derived from an erythropoietin receptor (EpoR), the ligand binding domain is not derived from the erythropoietin receptor (EpoR). In some embodiments, when the scaffold domain is derived from a leptin receptor (Ob-R), the ligand binding domain does not bind to leptin. In some embodiments, when the scaffold domain is derived from a leptin receptor (Ob-R), the ligand binding domain is not derived from the leptin receptor (Ob-R). In some embodiments, when the scaffold domain is derived from a growth hormone receptor (GHR), the ligand binding domain does not bind to growth hormone. In some embodiments, when the scaffold domain is derived from a growth hormone receptor (GHR), the ligand binding domain is not derived from the growth hormone receptor (GHR). In some embodiments, when the scaffold domain is derived from an epidermal growth factor receptor (EGFR), the ligand binding domain does not bind to epidermal growth factor (EGF). In some embodiments, when the scaffold domain is derived from an epidermal growth factor receptor (EGFR), the ligand binding domain is not derived from the epidermal growth factor receptor (EGFR). In some embodiments, when the scaffold domain is derived from a brain-derived neurotrophic receptor (Trk-B or p75^{NTR}) the ligand binding domain does not bind to brain-derived neurotrophic factor (BDNF). In some embodiments, when the scaffold domain is derived from a brain-derived neurotrophic receptor (Trk-B or p75^{NTR}), the ligand binding domain is not derived from the brain-derived neurotrophic receptor (Trk-B or p75^{NTR}).

In some embodiments, the ligand binding domain binds to a synthetic or designer ligand. In some embodiments, the ligand binding domain binds to a natural ligand. In some embodiments, the ligand binding domain binds to a ligand selected from the group consisting of a protein complex, a protein, a peptide, a nucleic acid, a small molecule, and a chemical agent. In some embodiments, the ligand is selected from the group consisting of an antigen, a cytokine, a survival factor, a chemokine, a hormone, a transmitter, a growth factor, extracellular matrix, and a death factor. In some embodiments, the ligand binding domain binds to a ligand displayed on a surface or expressed on a cell surface. In some embodiments, the ligand expressed on a cell surface is a tumor-associated antigen. The terms “tumor-associated antigen” and “cancer-associated-antigen” are used interchangeably herein and refer to a protein, carbohydrate, or other molecule whose expression is relatively restricted to tumor or cancer cells, but is also expressed on normal cells under certain conditions. The tumor-associated antigen may be any suitable such antigen known in the art. Exemplary tumor-associated antigens include, but are not limited to, MS4A3, VSTM1, LAT2, MLC1, CD131, GAPT, PRAM1, SLC22A16, SLC17A9, SPNS3, FLT3, CD33, CLEC12A, ADGRE2, IL3RA, CD117, CD93, IL1RAP, CD244, CCR1, LILRB2, PIEZO1, CD38, EMB, MYADM, LILRA2, CD300LF, CD70, 5T4, ADAM9, AFP, AXL, B7-H3, B7-H4, B7-H6, C4.4, CA6, Cadherin 3, Cadherin 6, CCR4, CD123, CD133, CD138, CD142, CD166, CD25, CD30, CD352, CD37, CD38, CD44, CD56, CD66e, CD71, CD74, CD79b, CD80, CEA, CEACAM5, Claudin18,2, cMet, CSPG4, CTLA, DLK1, DLL3, DR5, EGFR, ENPP3, EpCAM, EphA2, Ephrin A4, ETBR, FGFR2, FGFR3, FRalpha, FRb, GCC, GD2, GFRa4, gpA33, GPC3, gpNBM, GPRC5, HER2, IL-13Ra2, IL-8, IL-15, IL1RAP, Integrin aV, KIT, L1CAM, LAMP1, Lewis Y, LeY, LIV-1, LRRC, LY6E, MCSP, Mesothelin, MUC1, MUC16, MUC1C, NaPi2B, Nectin 4, NKG2D, NOTCH3, NY ESO 1, Ovarin, P-cadherin, pan-Erb2, PSCA, PSMA, PTK7, ROR1, S Aures, SCT, SLAMF7, SLITRK6, SSTR2, STEAP1, Survivin, TDGF1, TIM1, TROP2, and WT1.

In some embodiments, the ligand binding domain binds to a soluble ligand. In some embodiments, the soluble ligand may be a secreted cytokine, such as an immunosuppressive cytokine. The terms “immunosuppressive cytokine” and “anti-inflammatory cytokine” are used interchangeably herein to refer to cytokines that either inhibit pro-inflammatory cytokine synthesis or control pro-inflammatory cytokine-mediated cellular activities. Exemplary secreted cytokines include, but are not limited to, TGFbeta, IL-10, IL-6, IL-23, IL-8, CCL2, CXCL12, CXCL8, CXCL1, CXCL2, CXCL3, CXCL8, CXCL13, CCL5, CCL17, and CCL22. In some embodiments, the ligand binding domain binds to rapamycin. In some embodiments, the ligand binding domain binds to RR120. In some embodiments, the ligand binding domain binds to nicotine. In some embodiments, the ligand binding domain binds to an antigen. In some embodiments, the ligand binding domain binds to a cancer antigen. In some embodiments, the ligand binding domain binds to a tumor antigen. In some embodiments, the ligand binding domain binds to a pathogen antigen. In some embodiments, the ligand binding domain binds to a cell surface antigen expressed on a tumor cell. In some embodiments, the ligand binding domain binds to a cell surface antigen expressed on a tumor cell. In some embodiments, the ligand binding domain binds to a prostate-specific antigen (PSA). In some embodiments, the ligand binding domain binds to a peptide tag. In some embodiments, the ligand binding domain binds to a SunTag.

In some embodiments, the ligand binding domains of each receptor subunit are the same as one another. In some embodiments, the ligand binding domains of each receptor subunit are distinct from one another. In some embodiments, the ligand binding domain comprises an antibody, or antigen-binding fragment thereof. In some embodiments, the ligand binding domain comprises a single chain variable fragment (scFv), or a single-domain antibody (sdAb). In some embodiments, each of the ligand binding domains comprises a single chain variable fragment (scFv), optionally wherein each scFv specifically binds to a distinct epitope of the antigen. In some embodiments, the chimeric ligand receptor comprises two ligand binding domains, and wherein one ligand binding domain comprises an immunoglobulin heavy chain variable domain (V_{H}) and the second ligand binding domain comprises an immunoglobulin light chain variable domain (V_{L}). In some embodiments, the ligand binding domain comprises a single-domain VHH camelid antibody domain. In some embodiments, the ligand binding domain comprises a camelid heavy chain antibody domain (VHH) that homodimerizes in the presence of RR120. In some embodiments, the ligand binding domain comprises an immunoglobulin heavy chain variable domain (V_{H}) and an immunoglobulin light chain variable domain (V_{L}) of a nicotine antibody. In some embodiments, the ligand binding domain comprises a GCN4-specific scFv. In some embodiments, the ligand binding domain comprises an scFv that binds to the prostate-specific antigen (PSA). In some embodiments, the ligand binding domain comprises an FKBP-rapamycin binding protein (FRB) and an FK506 and rapamycin binding protein (FKBP). In some embodiments, the ligand binding domain comprises a leucine zipper domain. In some embodiments, the ligand binding domain comprises a PSD95-Dlg 1-zo-1 (PDZ) domain, a streptavidin domain and a streptavidin binding protein (SBP) domain, or a PYL domain and an ABI domain. In some embodiments, the ligand binding domain comprises a cyclophilin-Fas fusion protein (CyP-Fas) and a FK506 and rapamycin binding protein (FKBP). In some embodiments, the ligand binding domain comprises calcineurinA (CNA) and a FK506 and rapamycin binding protein (FKBP). In some embodiments, the ligand binding domain comprises gibberellin insensitive (GIA) and gibberellin insensitive dwarf1 (GID1). In some embodiments, the ligand binding domain comprises Snap-tag and Halo tag. In some embodiments, the ligand binding domain comprises T14-3-3-cdeltaC and C-Terminal peptides of PMA2 (CT52). Further description of suitable ligand binding domain can be found in the art, e.g. WO2017091546.

### Intracellular Signaling Domain

In some embodiments, the intracellular signaling domain is inert to native ligand binding of the scaffold domain. In some embodiments, the intracellular signaling domain does not comprise an endogenous intracellular signaling domain of the scaffold domain. In some embodiments, when the scaffold domain is derived from a cytokine receptor, the intracellular signaling domain is inert to the corresponding cytokine bound by the cytokine receptor. In some embodiments, when the scaffold domain is derived from a cytokine receptor, the intracellular signaling domain does not comprise an endogenous intracellular signaling domain of the cytokine receptor. In some embodiments, when the scaffold domain is derived from an erythropoietin receptor (EpoR), the intracellular signaling domain is inert to erythropoietin. In some embodiments, when the scaffold domain is derived from an erythropoietin receptor (EpoR), the intracellular signaling domain does not comprise an endogenous intracellular signaling domain of the erythropoietin receptor (EpoR). In some embodiments, when the scaffold domain is derived from a leptin receptor (Ob-R), the intracellular signaling domain is inert to leptin. In some embodiments, when the scaffold domain is derived from a leptin receptor (Ob-R), the intracellular signaling domain does not comprise an endogenous intracellular signaling domain of the leptin receptor (Ob-R). In some embodiments, when the scaffold domain is derived from a growth hormone receptor (GHR), the intracellular signaling domain is inert to growth hormone. In some embodiments, when the scaffold domain is derived from a growth hormone receptor (GHR), the intracellular signaling domain does not comprise an endogenous intracellular signaling domain of the growth hormone receptor (GHR). In some embodiments, when the scaffold domain is derived from an epidermal growth factor receptor (EGFR), the intracellular signaling domain is inert to epidermal growth factor. In some embodiments, when the scaffold domain is derived from an epidermal growth factor receptor (EGFR), the intracellular signaling domain does not comprise an endogenous intracellular signaling domain of the epidermal growth factor receptor (EGFR). In some embodiments, when the scaffold domain is derived from a brain-derived neurotrophic receptor (Trk-B or p75^{NTR}), the intracellular signaling domain is inert to brain-derived neurotrophic factor (BDNF). In some embodiments, when the scaffold domain is derived from a brain-derived neurotrophic receptor (Trk-B or p75^{NTR}), the intracellular signaling domain does not comprise an endogenous intracellular signaling domain of the brain-derived neurotrophic receptor (Trk-B or p75^{NTR}).

In some embodiments, the intracellular signaling domain induces downstream signaling via a JAK/STAT (Janus kinase/signal transducer and activator of transcription) signaling pathway, a MAPK (mitogen-activated protein kinase) signaling pathway, a PLCG (phospholipase C gamma) signaling pathway, or a PI3K/Akt (phosphatidylinositol 3-kinase/protein kinase B) signaling pathway. In some embodiments, the intracellular signaling domain is selected from the group consisting of an intracellular signal transduction domain of IL-6RB (interleukin 6 receptor B), an intracellular signal transduction domain of FGFR1 (fibroblast growth factor receptor 1), and an intracellular signal transduction domain of VEGFR2 (vascular endothelial growth factor receptor 2). In some embodiments, the intracellular signaling domain is an intracellular signal transduction domain of IL-6RB and induces downstream signaling via the JAK/STAT signaling pathway. In some embodiments, the intracellular signaling domain is an intracellular signal transduction domain of FGFR1 and induces downstream signaling via the MAPK signaling pathway. In some embodiments, the intracellular signaling domain is an intracellular signal transduction domain of VEGFR2 and induces downstream signaling via the PLCG signaling pathway. In some embodiments, the intracellular signaling domain is an intracellular signal transduction domain of VEGFR2 and induces downstream signaling via the PI3K/Akt signaling pathways. In some embodiments, the intracellular signaling domain is an intracellular signal transduction domain derived from an IL-2 signaling pathway, an IL-7 signaling pathway, or an IL-15 signaling pathway.

In some embodiments, the intracellular signaling domain comprises one or more modifications that modulate signaling activity of the intracellular signaling domain. In some embodiments, the modification comprises an amino acid insertion, an amino acid deletion, or an amino acid substitution. In some embodiments, the modification comprises an amino acid substitution. In some embodiments, the modification comprises a chemical modification. In some embodiments, the modification reduces negative feedback or reduces the cross-action of a secondary signaling pathway. In some embodiments, the modification comprises substitution of one or more tyrosine residues. In some embodiments, when the intracellular signaling domain is an intracellular signal transduction domain of IL-6RB, the intracellular signaling domain comprises a Y759A amino acid substitution. In some embodiments, when the intracellular signaling domain is an intracellular signal transduction domain of EGFR1, the intracellular signaling domain comprises a Y677F amino acid substitution. In some embodiments, when the intracellular signaling domain is an intracellular signal transduction domain of EGFR1, the intracellular signaling domain comprises a Y766F amino acid substitution. In some embodiments, when the intracellular signaling domain is an intracellular signal transduction domain of EGFR1, the intracellular signaling domain comprises a Y677F amino acid substitution and a Y766F amino acid substitution.

### Polynucleotides and Vectors

In another aspect, disclosed herein are isolated polynucleotides or sets of isolated polynucleotides encoding the chimeric ligand receptor. In some embodiments, the expression of the polynucleotide is under the control of a constitutively active promoter. In some embodiments, the expression of the polynucleotide is under the control of an inducible promoter. In some embodiments, the inducible promoter is a tetracycline-inducible promoter. Exemplary promoters for use in mammalian cells include but are not limited to cytomegalovirus (CMV) promoter, simian virus 40 (SV40) promoter, Rous sarcoma virus (RSV) promoter, elongation factor 1α (EF1α) promoter, and phosphoglycerate kinase (PGK) promoter.

In some embodiments, the polynucleotide encoding the chimeric ligand receptor is integrated into the genome by homologous recombination. In some embodiments, the polynucleotide encoding the chimeric ligand receptor is transiently transfected into the cells. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide. In some embodiments, polynucleotide encoding chimeric ligand receptor is delivered to a cell, cell line, or tissue using a CRISPR/Cas9 based system.

### Cells

In another aspect, disclosed herein are genetically engineered cells comprising the polynucleotide or expressing the chimeric ligand receptor. In some embodiments, the cell is a mammalian cell. In some embodiments, the mammalian cell is a primary cell. In some embodiments, the mammalian cell is a cell line. In some embodiments, the mammalian cell is a skin cell, a blood cell, a muscle cell, a bone cell, a neuronal cell, a fat cell, a liver cell, or a heart cell. In some embodiments, the cell is a stem cell. Exemplary stem cells include embryonic stem cells (ESCs), induced pluripotent stem cells (iPSCs), adult stem cells, and tissue-specific stem cells, such as hematopoietic stem cells (blood stem cells), mesenchymal stem cells (MSC), neural stem cells, epithelial stem cells, or skin stem cells. In some embodiments, the cell is an immune cell. Exemplary immune cells include T cells (e.g., helper T cells, cytotoxic T cells, memory T cells, regulatory T cells, natural killer T cells, and gamma delta T cells), B cells, natural killer (NK) cells, dendritic cells, macrophages, monocytes, innate lymphoid cells, mast cells, eosinophils, basophils, and neutrophils. In some embodiments, the T cell is a cytokine-induced killer cell. In some embodiments, the cell is a neuronal cell. Exemplary neuronal cells include neural progenitor cells, neurons (e.g., sensory neurons, motor neurons, cholinergic neurons, GABAergic neurons, glutamatergic neurons, dopaminergic neurons, or serotonergic neurons), astrocytes, oligodendrocytes, and microglia.

In some embodiments, the genetically engineered cell further comprises an engineered transgene. In some embodiments, the transgene comprises a synthetic promoter operably linked to a polynucleotide comprising a nucleic acid sequence encoding a target product. In some embodiments, the synthetic promoter is responsive to intracellular signaling from the chimeric ligand receptor. In some embodiments, the target product is selected from the group consisting of a therapeutic molecule (e.g., enzymes or antibodies), a prophylactic molecule, and a diagnostic molecule. In some embodiments, the target product is glucagon-like peptide 1, luciferase, secreted alkaline phosphatase (SEAP), or insulin. In some embodiment, the target product is a chimeric antigen receptor (CAR), chimeric immunoreceptor (e.g., a chimeric T cell receptor), an artificial immunoreceptor (e.g., an artificial T cell receptor), a synthetic immunoreceptor (e.g., a synthetic T cell receptor), an engineered immunoreceptor (e.g., an engineered T cell receptor), or an antibody-coupled receptor (e.g., an antibody-coupled T cell receptor). In some embodiments, the target product is a proinflammatory cytokine, such as IL-2, IL-7, IL-12, IL-15, IL-18, and IL-21. In some embodiments, the target product is CD40L. In some embodiments, the target product is 4-1BBL.

In some embodiments, the genetically engineered cell comprises two or more chimeric ligand receptors. In some embodiments, the chimeric ligand receptors are each distinct from one another. In some embodiments, the chimeric ligand receptors each bind a different ligand. In some embodiments, the genetically engineered cell further comprises two or more engineered transgenes. In some embodiments, each transgene comprises a synthetic promoter operably linked to a polynucleotide comprising a nucleic acid sequence encoding a target product. In some embodiments, each synthetic promoter is responsive to intracellular signaling from a distinct chimeric ligand receptor from the two or more chimeric ligand receptors expressed on the cell. In some embodiments, each target product is independently selected from the group consisting of a therapeutic molecule, a prophylactic molecule, and a diagnostic molecule.

### Methods

In another aspect, disclosed herein are methods of contacting the chimeric ligand receptor or the genetically engineered cell with a biological tissue or biological fluid. In some embodiments, the biological tissue or biological fluid is in a subject or is obtained from a subject. In some embodiments, the subject has been diagnosed with, is at risk of developing, or is suspected of having a medical condition. In some embodiments, the medical condition is a cancer or inflammatory condition.

In another aspect, disclosed herein are methods of activating a signaling pathway. The method comprises contacting the chimeric ligand receptor or the genetically engineered cell with a cognate ligand under conditions suitable for the chimeric ligand receptor to bind the cognate ligand, wherein binding of the cognate ligand with the chimeric ligand receptor induces a conformational reorganization of the multimerized scaffold domains that activates the intracellular signaling domains. In some embodiments, the method further comprises administering the cognate ligand to a surface of a cell.

In another aspect, disclosed herein are methods of producing a genetically engineered cell expressing a chimeric ligand receptor. The method comprises: synthesizing a chimeric ligand receptor expression vector encoding a chimeric ligand receptor comprising a scaffold domain capable of muitimerizing and comprising an extracellular domain and a transmembrane domain, a ligand binding domain operably linked to the extracellular binding domain of the scaffold domain, and an intracellular signaling domain operably linked to the transmembrane domain of the scaffold domain, by fusing a first nucleic acid encoding the ligand binding domain to a second nucleic acid encoding the scaffold domain, and fusing the second nucleic acid with a third nucleic acid encoding the intracellular signaling domain; transfecting the chimeric ligand receptor expression vector into a cell; and inducing expression of the chimeric ligand receptor in the cell. In some embodiments, the method comprises transfecting the isolated polynucleotide or set of isolated polynucleotides or the vector or set of vectors into a cell; and inducing expression of the chimeric ligand receptor in the cell. In some embodiments, the method further comprises transfecting into the cell an isolated polynucleotide comprising a synthetic promoter operably linked to a nucleic acid sequence encoding a target product. In some embodiments, the synthetic promoter is responsive to intracellular signaling from the chimeric ligand receptor. In some embodiments, inducing expression of the chimeric ligand receptor comprises culturing the cell under conditions suitable for the cell to express the chimeric ligand receptor on a cell membrane of the cell.

### EXAMPLES

The following are examples of methods and compositions of the present disclosure. It is understood that various other embodiments may be practiced, given the general description provided herein.

Below are examples of specific embodiments for carrying out the claimed invention. The examples are offered for illustrative purposes only, and are not intended to limit the scope of the present disclosure in any way. Efforts have been made to ensure accuracy with respect to numbers used (e.g., amounts, temperatures, etc.), but some experimental error and deviation should, of course, be allowed for.

The practice of the present disclosure will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa: Mack Publishing Company, 1990); Carey and Sundberg Advanced Organic Chemistry 3^{rd} Ed. (Plenum Press) Vols A and B(1992).

Materials and Methods

HEK-293 cell culture and transduction. HEK-Blue-IL6 reporter cells were purchased from InvivoGen. The cells express the reporter SEAP upon activation of the IL6/STAT3 signaling pathway. GEMS_{RR120} virus was used for transduction into HEK-Blue-IL 6 cells. 50,000 cells per well were seeded in a 96 well plate and untreated or treated with 100 ng/mL IL6 (positive control) or with 100 ng/mL of RR120 in complete DMEM media. Cells were incubated at 37 C for 20-24 hrs.

SEAP measurement. SEAP readout was measured using Quanti-Blue solution (InvivoGen). After incubating assay supernatant with Quanti-Blue for 1-2 hours, the plate was read using a spectrophotometer at 655 nm to determine SEAP secretion.

### Example 1

### Validation of the GEMS Receptor Scaffold

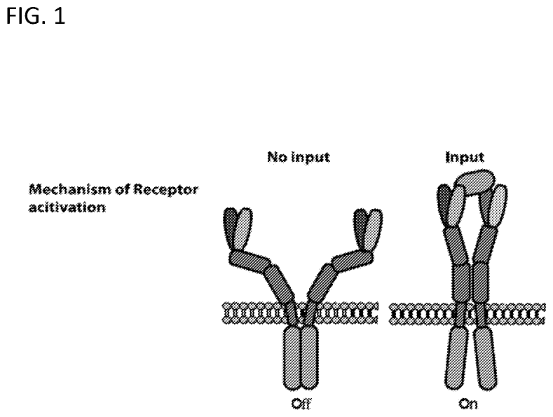

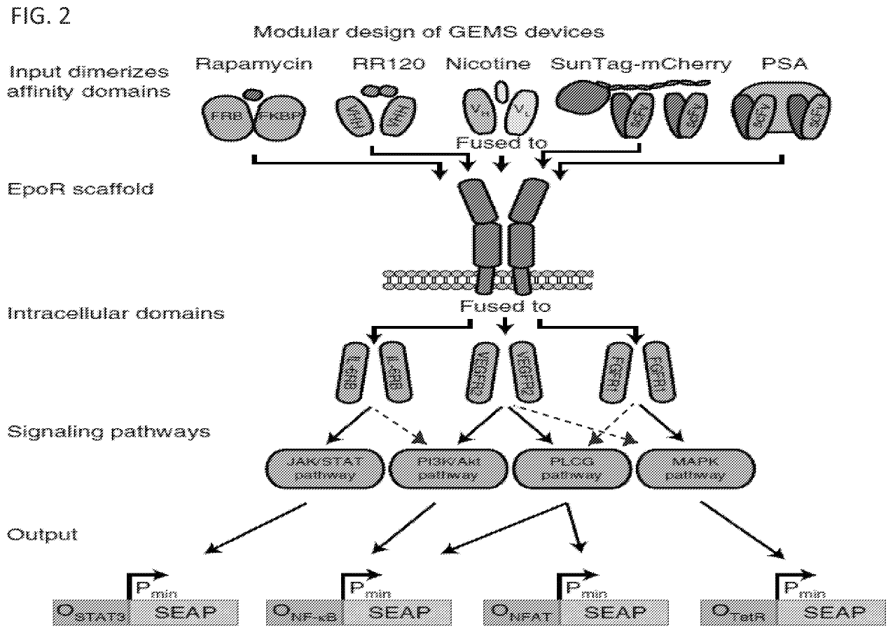

The GEMS system functions by the mechanism of dimerization of extracellular receptor domains, which causes activation of intracellular signaling domains (FIG. 1). Cytokine receptors have a modular structure that tolerates the combination of intracellular and extracellular domains of different receptors to produce functional chimeras (See Arber C et al., Curr. Opin. Biotechnol., 2017, 47:92-101; Kawahara M & Nagamune T, Curr. Opin. Chem. Eng., 2012, 1:411-417). Inactive EpoR dimers are locked by transmembrane helix interactions in a conformation that prevents downstream signaling (See Seubert N et al., Mol. Cell, 2003, 12:1239-1250). Ligand binding to the receptors is thought to rotate each receptor subunit around its own axis and is likely accompanied by an increase in the distance between intracellular domains. The combination of these effects triggers downstream signaling (See Pang X & Zhou H X, PLoS Comput. Biol., 2012, 8:e1002427). By introducing a mutation into the erythropoietin receptor, it can be rendered inert to erythropoietin and can then be fused to affinity domains such as antibody fragments that dimerize in the presence of target molecules. As shown in FIG. 2, the EpoR transmembrane domain may be fused to the intracellular signal transduction domains of IL-6RB (interleukin 6 receptor B), FGFR1 (fibroblast growth factor receptor 1), or VEGFR2 (vascular endothelial growth factor receptor 2). Once activated, these intracellular domains can induce downstream signaling via JAK/STAT (Janus kinase/signal transducer and activator of transcription; induced by IL-6RB), MAPK (mitogen-activated protein kinase; induced by FGFR1) and PLCG (phospholipase C gamma; induced by VEGFR2), as well as PI3K/Akt (phosphatidylinositol 3-kinase/protein kinase B; induced by VEGFR2). Minimal promoters that are selectively responsive to the indicated pathways may be used to rewire signaling to transgene expression.

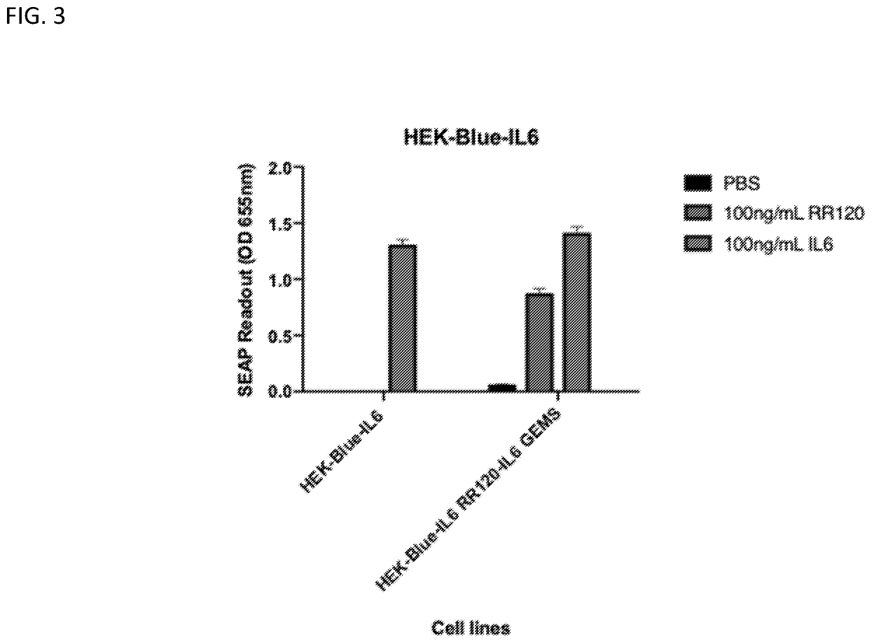

The GEMS architecture was validated in the context of an RR120 binding extracellular domain and IL-6 intracellular signaling domain (FIG. 3). This model system provides a positive control for future GEMS experiments in which the intracellular signaling domain and the extracellular binding domain will be switched for inputs and signaling pathways of interest.

### Sequences

| SEQ ID NO: | SEQUENCE | DESCRIPTION |
|---|---|---|
| 1 | MDKLRVPLWPRVGPLCLLLAGAAWAPSPSLPDPKFESKAALLASRGSEELLCF | EpoR precursor |
| | TQRLEDLVCFWEEAASSGMDFNYSFSYQLEGESRKSCSLHQAPTVRGSVRFWC | (mouse) |
| | SLPTADTSSFVPLELQVTEASGSPRYHRIIHINEVVLLDAPAGLLARRAEEGS | |
| | HVVLRWLPPPGAPMTTHIRYEVDVSAGNRAGGTQRVEVLEGRTECVLSNLRGG | |
| | TRYTFAVRARMAEPSFSGFWSAWSEPASLLTASDLDPLILTLSLILVLISLLL | |
| | TVLALLSHRRTLQQKIWPGIPSPESEFEGLFTTHKGNFQLWLLQRDGCLWWSP | |
| | GSSFPEDPPAHLEVLSEPRWAVTQAGDPGADDEGPLLEPVGSEHAQDTYLVLD | |
| | KWLLPRTPCSENLSGPGGSVDPVTMDEASETSSCPSDLASKPRPEGTSPSSFE | |
| | YTILDPSSQLLCPRALPPELPPTPPHLKYLYLVVSDSGISTDYSSGGSQGVHG | |
| | DSSDGPYSHPYENSLVPDSEPLHPGYVACS | |
| 2 | APSPSLPDPKFESKAALLASRGSEELLCFTQRLEDLVCFWEEAASSGMDFNYS | Mature EpoR |
| | FSYQLEGESRKSCSLHQAPTVRGSVRFWCSLPTADTSSFVPLELQVTEASGSP | (mouse) |
| | RYHRIIHINEVVLLDAPAGLLARRAEEGSHVVLRWLPPPGAPMTTHIRYEVDV | |
| | SAGNRAGGTQRVEVLEGRTECVLSNLRGGTRYTFAVRARMAEPSFSGFWSAWS | |
| | EPASLLTASDLDPLILTLSLILVLISLLLTVLALLSHRRTLQQKIWPGIPSPE | |
| | SEFEGLFTTHKGNFQLWLLQRDGCLWWSPGSSFPEDPPAHLEVLSEPRWAVTQ | |
| | AGDPGADDEGPLLEPVGSEHAQDTYLVLDKWLLPRTPCSENLSGPGGSVDPVT | |
| | MDEASETSSCPSDLASKPRPEGTSPSSFEYTILDPSSQLLCPRALPPELPPTP | |
| | PHLKYLYLVVSDSGISTDYSSGGSQGVHGDSSDGPYSHPYENSLVPDSEPLHP | |
| | GYVACS | |
| 3 | MDKLVPLWPRVGPLCLLLAGAAWAPSPSLPDPKFESKAALLASRGSEELLCFT | Isoform EpoR-S; |
| | QRLEDLVCFWEEAASSGMDFNYSFSYQLEGESRKSCSLHQAPTVRGSVRFWCS | soluble form |
| | LPTADTSSFVPLELQVTEASGSPRYHRIIHINEVVLLDAPAGLLARRAEEGSH | (mouse) |
| | VVLRWLPPPGAPMTTHIRYEVDVSAGNRAGGTQRVEVLEGRTECVLSNLRGGT | |
| | RYTFAVRARMAEPSFSGFWSAWSEPASLLTASGEALVPRGAGGAGPNTRQTP | |
| 4 | MDHLGASLWPQVGSLCLLLAGAAWAPPPNLPDPKFESKAALLAARGPEELLCF | EpoR precursor |
| | TERLEDLVCFWEEAASAGVGPGNYSFSYQLEDEPWKLCRLHQAPTARGAVRFW | (human) |
| | CSLPTADTSSFVPLELRVTAASGAPRYHRVIHINEVVLLDAPVGLVARLADES | |
| | GHVVLRWLPPPETPMTSHIRYEVDVSAGNGAGSVQRVEILEGRTECVLSNLRG | |
| | RTRYTFAVRARMAEPSFGGFWSAWSEPVSLLTPSDLDPLILTLSLILVVILVL | |
| | LTVLALLSHRRALKQKIWPGIPSPESEFEGLFTTHKGNFQLWLYQNDGCLWWS | |
| | PCTPFTEDPPASLEVLSERCWGTMQAVEPGTDDEGPLLEPVGSEHAQDTYLVL | |
| | DKWLLPRNPPSEDLPGPGGSVDIVAMDEGSEASSCSSALASKPSPEGASAASF | |
| | EYTILDPSSQLLRPWTLCPELPPTPPHLKYLYLVVSDSGISTDYSSGDSQGAQ | |
| | GGLSDGPYSNPYENSLIPAAEPLPPSYVACS | |
| 5 | APPNLPDPKFESKAALLAARGPEELLCFTERLEDLVCFWEEAASAGVGPGNYS | Mature EpoR |
| | FSYQLEDEPWKLCRLHQAPTARGAVRFWCSLPTADTSSFVPLELRVTAASGAP | (human) |
| | RYHRVIHINEVVLLDAPVGLVARLADESGHVVLRWLPPPETPMTSHIRYEVDV | |
| | SAGNGAGSVQRVEILEGRTECVLSNLRGRTRYTFAVRARMAEPSFGGFWSAWS | |
| | EPVSLLTPSDLDPLILTLSLILVVILVLLTVLALLSHRRALKQKIWPGIPSPE | |
| | SEFEGLFTTHKGNFQLWLYQNDGCLWWSPCTPFTEDPPASLEVLSERCWGTMQ | |
| | AVEPGTDDEGPLLEPVGSEHAQDTYLVLDKWLLPRNPPSEDLPGPGGSVDIVA | |
| | MDEGSEASSCSSALASKPSPEGASAASFEYTILDPSSQLLRPWTLCPELPPTP | |
| | PHLKYLYLVVSDSGISTDYSSGDSQGAQGGLSDGPYSNPYENSLIPAAEPLPP | |
| | SYVACS | |
| 6 | MDHLGASLWPQVGSLCLLLAGAAWAPPPNLPDPKFESKAALLAARGPEELLCF | Isoform EpoR-S; |
| | TERLEDLVCFWEEAASAGVGPGNYSFSYQLEDEPWKLCRLHQAPTARGAVRFW | soluble form |
| | CSLPTADTSSFVPLELRVTAASGAPRYHRVIHINEVVLLDAPVGLVARLADES | (human) |
| | GHVVLRWLPPPETPMTSHIRYEVDVSAGNGAGSVQRGTVFLSPDWLSSTRARP | |
| | HVIYFCLLRVPRPDSAPRWRSWRAAPSVC | |
| 7 | MDHLGASLWPQVGSLCLLLAGAAWAPPPNLPDPKFESKAALLAARGPEELLCF | Isoform EpoR-T; |
| | TERLEDLVCFWEEAASAGVGPGNYSFSYQLEDEPWKLCRLHQAPTARGAVRFW | truncated form |
| | CSLPTADTSSFVPLELRVTAASGAPRYHRVIHINEVVLLDAPVGLVARLADES | (human) |
| | GHVVLRWLPPPETPMTSHIRYEVDVSAGNGAGSVQRVEILEGRTECVLSNLRG | |
| | RTRYTFAVRARMAEPSFGGFWSAWSEPVSLLTPSDLDPLILTLSLILVVILVL | |
| | LTVLALLSHRRALKQKIWPGIPSPESEFEGLFTTHKGNFQVGGLVVPSVPGLP | |
| | CFLQPNCRPL | |
| 8 | APSPSLPDPKFESKAALLASRGSEELLCFTQRLEDLVCFWEEAASSGMDFNYS | Minimal EpoR |
| | FSYQLEGESRKSCSLHQAPTVRGSVRFWCSLPTADTSSFVPLELQVTEASGSP | scaffold domain |
| | RYHRIIHINEVVLLDAPAGLLARRAEEGSHVVLRWLPPPGAPMTTHIRYEVDV | |
| | SAGNRAGGTQRVEVLEGRTECVLSNLRGGTRYTFAVRARMAEPSFSGFWSAWS | |
| | EPASLLTASDLDPLILTLSLILVLISLLLTVLALLS | |
| 9 | APSPSLPDPKFESKAALLASRGSEELLCFTQRLEDLVCFWEEAASSGMDFNYS | Minimal EpoR |
| | FSYQLEGESRKSCSLHQAPTVRGSVRFWCSLPTADTSSFVPLELQVTEASGSP | extracellular |
| | RYHRIIHINEVVLLDAPAGLLARRAEEGSHVVLRWLPPPGAPMTTHIRYEVDV | domain |
| | SAGNRAGGTQRVEVLEGRTECVLSNLRGGTRYTFAVRARMAEPSFSGFWSAWS | |
| | EPASLLTASDLDP | |
| 10 | LILTLSLILVLISLLLTVLALLS | Minimal EpoR |
| | | transmembrane |
| | | domain |
| 11 | MICQKFCVVLLHWEFIYVITAFNLSYPITPWRFKLSCMPPNSTYDYFLLPAGL | leptin receptor |
| | SKNTSNSNGHYETAVEPKFNSSGTHFSNLSKTTFHCCFRSEQDRNCSLCADNI | (Ob-R) |
| | EGKTFVSTVNSLVFQQIDANWNIQCWLKGDLKLFICYVESLFKNLFRNYNYKV | |
| | HLLYVLPEVLEDSPLVPQKGSFQMVHCNCSVHECCECLVPVPTAKLNDTLLMC | |
| | LKITSGGVIFQSPLMSVQPINMVKPDPPLGLHMEITDDGNLKISWSSPPLVPF | |
| | PLQYQVKYSENSTTVIREADKIVSATSLLVDSILPGSSYEVQVRGKRLDGPGI | |
| | WSDWSTPRVFTTQDVIYFPPKILTSVGSNVSFHCIYKKENKIVPSKEIVWWMN | |
| | LAEKIPQSQYDVVSDHVSKVTFFNLNETKPRGKFTYDAVYCCNEHECHHRYAE | |
| | LYVIDVNINISCETDGYLTKMTCRWSTSTIQSLAESTLQLRYHRSSLYCSDIP | |
| | SIHPISEPKDCYLQSDGFYECIFQPIFLLSGYTMWIRINHSLGSLDSPPTCVL | |
| | PDSVVKPLPPSSVKAEITINIGLLKISWEKPVFPENNLQFQIRYGLSGKEVQW | |
| | KMYEVYDAKSKSVSLPVPDLCAVYAVQVRCKRLDGLGYWSNWSNPAYTVVMDI | |
| | KVPMRGPEFWRIINGDTMKKEKNVTLLWKPLMKNDSLCSVQRYVINHHTSCNG | |
| | TWSEDVGNHTKFTFLWTEQAHTVTVLAINSIGASVANFNLTFSWPMSKVNIVQ | |
| | SLSAYPLNSSCVIVSWILSPSDYKLMYFIIEWKNLNEDGEIKWLRISSSVKKY | |
| | YIHDHFIPIEKYQFSLYPIFMEGVGKPKIINSFTQDDIEKHQSDAGLYVIVPV | |
| | IISSSILLLGTLLISHQRMKKLFWEDVPNPKNCSWAQGLNFQKPETFEHLFIK | |
| | HTASVTCGPLLLEPETISEDISVDTSWKNKDEMMPTTVVSLLSTTDLEKGSVC | |
| | ISDQFNSVNFSEAEGTEVTYESESQRQPFVKYATLISNSKPSETGEEQGLINS | |
| | SVTKCFSSKNSPLKDSFSNSSWEIEAQAFFILSDQHPNIISPHLTFSEGLDEL | |
| | LKLEGNFPEENNDKKSIYYLGVTSIKKRESGVLLTDKSRVSCPFPAPCLFTDI | |
| | RVLQDSCSHFVENNINLGTSSKKTFASYMPQFQTCSTQTHKIMENKMCDLTV | |
| 12 | FNLSYPITPWRFKLSCMPPNSTYDYFLLPAGLSKNTSNSGHYETAVEPKFNSS | leptin receptor |
| | GTHFSNLSKTTFHCCFRSEQDRNCSLCADNIEGKTFVSTVNSLVFQQIDANWN | (Ob-R) |
| | IQCWLKGDLKLFICYVESLFKNLFRNYNYKVHLLYVLPEVLESDPLVPQKGSF | |
| | QMVHCNCSVHECCECLVPVPTAKLNDTLLMCLKITSGGVIFQSPLMSVQPINM | |
| | VKPDPPLGLHMEITDDGNLKISWSSPPLVPFPLQYQVKYSENSTTVIREADKI | |
| | VSATSLLVDSILPGSSYEVQVRGKRLDGPGIWSDWSTPRVFTTQDVIYFPPKI | |
| | LTSVGSNVSFHCIYKKENKIVPSKEIVWWMNLAEKIPQSQYDVVSDHVSKVTF | |
| | FNLNETKPRGKFTYDAVYCCNEHECHHRYAELYVIDVNINISCETDGYLTKMT | |
| | CRWSTSTIQSLAESTLQLRYHRSSLYCSDIPSIHPISEPKDCYLQSDGYFECI | |
| | FQPIFLLSGYTMWIRINHSLGSLDSPPTCVLPDSVVKPLPPSSVKAEITINIG | |
| | LLKISWEKPVFPENNLQFQIRYGLSGKEVQWKMYEVYDAKSKSVSLPVPDLCA | |
| | VYAVQVRCKRLDGLGYWSNWSNPAYTVVMDIKVPMRGPEFWRIINGDTMKKEK | |
| | NVTLLWKPLMKNDSLCSVQRYVINHYHTSCNGTWSEDVGNHTKFTFLWTEQAH | |
| | TVTVLAINSIGASVANFNLTFSWPMSKVNIVQSLSAYPLSNNCVIVSWILSPS | |
| | DYKLMYFIIEWKNLNEDGEIKWLRISSSVKKYYIHDHFIPIEKYQFSLYPIFM | |
| | EGVGKPKIINSFTQDDIEKHQSDAGLYVIVPVIISSSILLLGTLLI | |
| 13 | FNLSYPITPWRFKLSCMPPNSTYDYFLLPAGLSKNTSNSNGHYETAVEPKFNS | leptin receptor |
| | SGTHFSNLSKTTFHCCFRSEQDRNCSLCADNIEGKTFVSTVNSLVFQQIDANW | (Ob-R) |
| | IQCWLKGDLKLFICYVESLFKNLFRNYNYKVHLLYVLPEVLEDSPLVPQKGSF | |
| | QMVHCNCSVHECCECLVPVPTAKLNDTLLMCLKITSGGVIFQSPLSMVQPINM | |
| | VKPDPPLGLHMEITDDGNLKISWSSPPLVPFPLQYQVKYSENSTTVIREADKI | |
| | VSATSLLVDSILPGGYEVQVRGKRLDGPGIWSDWSTPRVFTTQDVIYFPPKIL | |
| | TSVGSNVSFHCIYKKENKIVPSKEIVWWMNLAEKIPQSQYDVVSDHVSKVTFF | |
| | NLNETKPRGKFTYDAVYCCNEHECHHRYAELYVIDVNINISCETDGYLTKMTC | |
| | RWSTSTIQSLAESTLQLRYHRSSLYCSDIPSIHPISEPKDCYLQSDGFYECIF | |
| | QPIFLLSGYTMWIRINHSLGSLDSPPTCVLPDSVVKPLPPSSVKAEITINIGL | |
| | LKISWEKPVFPENNLQFQIRYGLSGKEVQWKMYEVYDAKSKSVSLPVPDLCAV | |
| | YAVQVRCKRLDGLGYWSNWSNPAYTVVMDIKVPMRGPEFWRIIGDTMKKEKNV | |
| | TLLWKPLMKNDSLCSVQRYVINHHTSCNGTWSEDVGNHTKFTFLWTEQAHTVT | |
| | VLAINSIGASVANFNLTFSWPMSKVNIVQSLSAYPLNSSCVIVSWILSPSDYK | |
| | LMYFIIEWKNLNEDGEIKWLRISSSVKKYYIHDHFIPIEKYQFSLYPIFMEGV | |
| | GKPKIINSFTQDDIEKHQSD | |
| 14 | AGLYVIVPVIISSSILLLGTLLI | |
| 15 | MDLWQLLLTLALAGSSDAFSGSEATAAILSRAPWSLQSVNPGLKTMSSKEPKF | Growth hormone |
| | TKCRSPERETFSCHWTDEVHHGTKNLGPIQLFYTRRNTQEWTQEWKECPDYVS | receptor (GHR) |
| | AGENSCYFNSSFTSIWIPYCIKLTSNGGTVDEKCFSVDEIVQPDPPIALNWTL | |
| | LNVSLTGIHADIQVRWEAPRNADIQKGWMVLEYELQYKEVNETKWKMMDPILT | |
| | TSVPVYSLKVDKEYEVRVRSKQRNSGNYGEFSEVLYVTLPQMSQFTCEEDFYF | |
| | PWLLIIIFGIFGLTVMLFVFLFSKQQRIKMLILPPVPVPKIKGIDPDLLKEGK | |
| | LEEVNTILAIHDSYKPEFHSDDSWVEFIELDIDEPDEKTEESDTDRLLSSDHE | |
| | KSHSNLGVKDGDSGRTSCCEPDILETDFNANDIHEGTSEVAQPQRLKGEADLL | |
| | CLDQKNQNNSPYHDACPATQQPSVIQAEKNKPQPLPTEGAESTHQAAHIQLSN | |
| | PSSLSNIDFYAQVSDITPAGSVVLSPGQKNKAGMSQCDMHPEMVSLCQENFLM | |
| | DNAYFCEADAKKCIPVAPHIKVESHIQPSLNQEDIYITTESLTTAAGRPGTGE | |
| | HVPGSEMPVPDYTSIHIVQSPQGLILNATALPLPDKEFLSSCGYVSTDQLNKI | |
| | MP | |
| 16 | FSGSEATAAILSRAPWSLQSVNPGLKTNSSKEPKFTKCRSPERETFSCHWTDE | Growth hormone |
| | VHHGTKNLGPIQLFYTRRNTQEWTQEWKECPDYVSAGENSCYFNSSFTSIWIP | receptor (GHR) |
| | YCIKLTSNGGTVDEKCFSVDEIVQPDPPIALNWTLLNVSLTGIHADIQVRWEA | |
| | PRNADIQKGWMVLEYELQYKEVNETKWKMMDPILTTSVPVYSLKVDKEYEVRV | |
| | RSKQRNSGNYGEFSEVLYVTLPQMSQFTCEEDFYFPWLLIIIFGIFGLTVMLF | |
| | VFLFS | |
| 17 | FSGSEATAAILSRAPWSLQSVNPGLKTNSSKEPKFTKVRSPERETFSCHWTDE | Growth hormone |
| | VHHGTKNLGPIQLFYTRRNTQEWTQEWKECPDYVSAGENSCYFNSSFTSIWIP | receptor (GHR) |
| | YCIKLTSNGGTVDEKCFSVDEIVQPDPPIALNWTLLNVSLTGIHADIQVRWEA | |
| | PRNADIQKGWMVLEYELQYKEVNETKWKMMDPILTTSVPVYSLKVKDEYEVRV | |
| | RSKQRNSGNYGEFSEVLYVTLPQMSQFTCEEDFY | |
| 18 | FPWLLIIIFGIFGLTVMLFVFLFS | |
| 19 | MRPSGTAGAALLALLAALCPASRALEEKKVCQGTSNKLTQLGTFEDHFLSLQR | epidermal |
| | MFNNCEVVLGNLEITYVQRNYDLSFLKTIQEVAGYVLIALNTVERIPLENLQI | growth factor |
| | IRGNMYYENSYALAVLSNYDANKTGLKELPMRNLQEILHGAVRFSNNPALCNV | receptor (EGFR) |
| | ESIQWRDIVSSDFLSNMSMDFQNHLGSCQKCDPSCPNGSCWGAGEENCQKLTK | |
| | IICAQQCSGRCRGKSPSDCCHNQCAAGCTGPRESDCLVCRKFRDEATCKDTCP | |
| | PLMLYNPTTYQMDVNPEGKYSFGATCVKKCPRNYVVTDHGSCVRACGADSYEM | |
| | EEDGVRKCKKCEGPCRKVCNGIGIGEFKDSLSINATNIKHFKNCTSISGDLHI | |
| | LPVAFRGDSFTHTPPLDPQELDILKTVKEITGFLLIQAWPENRTDLHAFENLE | |
| | IIRGRTKQHGQFSLAVVSLNITSLGLRSLKEISDGDVIISGNKNLCYANTINW | |
| | KKLFGTSGQKTKIISNRGENSCKATGQVCHALCSPEGCWGPEPRDCVSCRNVS | |
| | RGRECVDKCNLLEGEPREFVENSECIQCHPECLPQAMNITCTGRGPDNCIQCA | |
| | HYIDGPHCVKTCPAGVMGENNTLVWKADAGHVCHLCHPNCTYGCTGPGLEGCP | |
| | TNGPKIPSIATGMVGALLLLLVVALGIGLFMRRRHIVRKRTLRRLLQERELVE | |
| | PLTPSGEAPNQALLRILKETEFKKIKVLGSGAFGTVYKGLWIPEGEKVKIPVA | |
| | IKELREATSPKANKEILDEAYVMASVDNPHVCRLLGICLTSTVQLITQLMPFG | |
| | CLLDYVREHKDNIGSQYLLNWCVQIAKGMNYLEDRRLVHRDLAARNVLVKTPQ | |
| | HVKITDFGLAKLLGAEEKEYHAEGGKVPIKWMALESILHRIYTHQSDVWSYGV | |
| | TVWELMTFGSKPYDGIPASEISSILEKGERLPQPPICTIDVYMIMVKCWMIDA | |
| | DSRPKFRELIIEFSKMARDPQRYLVIQGDERMHLPSPTDSNFYRALMDEEMDD | |
| | VVDADEYLIPQQGFFSSPSTSRTPLLSSLSATSNNSTVACIDRNGLQSCPIKE | |
| | DSFLQRYSSDPTGALTEDSIDDTFLPVPEYINQSVPKRPAGSVQNPVYHNQPL | |
| | NPAPSRDPHYQDPHSTAVGNPEYLNTVQPTCVNSTFDSPAHWAQKGSHQISLD | |
| | NPDYQQDFFPKEAKPNGIFKGSTAENAEYLRVAPQSSEFIGA | |
| 20 | LEEKKVCQGTSNKLTQLGTFEDHFLSLQRMFNNCEVVLGNLEITYVQRNYDLS | epidermal |
| | FLKTIQEVAGYVLIALNTVERIPLENLQIIRGNMYYENSYALAVLSNYDANKT | growth factor |
| | GLKELPMRNLQEILHGAVRFSNNPALCNVESIQWRDIVSSDFLSNMSMDFQNH | receptor(EGFR) |
| | LGSCQKCDPSCPNGSCWGAGEENCQKLTKIICAQQCSGRCRGKSPSDCCHNQC | |
| | AAGCTGPRESDCLVCRKFRDEATCKDTCPPLMLYNPTTYQMDVNPEGKYSFGA | |
| | TCVKKCPRNYVVTDHGSCVRACGADSYEMEEDGVRKCKKCEGPCRKVCNGIGI | |
| | GEFKDSLSINATNIKHFKNCTSISGDLHILPVAFRGDSFTHTPPLDPQELDIL | |
| | KTVKEITGFLLIQAWPENRTDLHAFENLEIIRGRTKQHGQFSLAVVSLNITSL | |
| | GLRSLKEISDGDVIISGNKNLVYANTINWKKLFGTSGQKTKIISNRGENSCKA | |
| | TGQVCHALCSPEGCWGPEPRDCVSCRNVSRGRECVDKCNLLEGEPREFVENSE | |
| | CIQCHPECLPQAMNITCTGRGPDNCIQCAHYIDGPHCVKTCPAGVMGENNTLV | |
| | WKYADAGHVCHLCHPNCTYGCTGPGLEGCPTNGPKIPSIATGMVGALLLLLVV | |
| | ALGIGLFM | |
| 21 | LEEKKVCQGTSNKLTQLGTFEDHFLSLQRMFNNCEVVLGNLEITYVQRNYDLS | epidermal |
| | FLKTIQEVAGYVLIALNTVERIPLENLQIIRGNMYYENSYALAVLSNYDANKT | growth factor |
| | GLKELPMRNLQEILHGAVRFSNNPALCNVESIQWRDIVSSDFLSNMSMDFQNH | receptor(EGFR) |
| | LGSCQKCDPSCPNGSCWGAGEENCQKLTKIICAQQCSGRCRGKSPSDCCHNQC | |
| | AAGCTGPRESDCLVCRKFRDEATCKDTCPPLMLYNPTTYQMDVNPEGKYSFGA | |
| | TCVKKCPRNYVVTDHGSCVRACGADSYEMEEDGVRKCKKCEGPCRKVCNGIGI | |
| | GEFKDSLSINATNIKHFKNCTSISGDLHILPVAFRGDSFTHTPPLDPQELDIL | |
| | KTVKEITGFLLIQAWPENRTDLHAFENLEIIRGRTKQHGQFSLAVVSLNITSL | |
| | GLRSLKEISDGDVIISGNKNLCYANTINWKKLFGTSGQKTKIISNRGENSCKA | |
| | TGQVCHALCSPEGCWGPEPRDCVSCRNVSRGRECVDKCNLLEGEPREFVENSE | |
| | CIQCHPECLPQAMNITCTGRGPDNCIQCAHYIDGPHCVKTCPAGVMGENNTLV | |
| | WKYADAGHVCHLCHPNCTYGCTGPGLEGCPTNGPKIPS | |
| 22 | IATGMVGALLLLLVVALGIGLFM | |
| 23 | MSSWIRWHGPAMARLWGFCWLVVGFWRAAFACPTSCKCSASRIWCSDPSPGIV | Trk-B |
| | AFPRLEPNSVDPENITEIFIANQKRLEIINEDDVEAYVGLRNLTIVDSGLKFV | |
| | AHKAFLKNSNLQHINFTRNKLTSLSRKHFRHLDLSELILVGNPFTCSCDIMWI | |
| | KTLQEAKSSPDTQDLYCLNESSKNIPLANLQIPNCGLPSANLAAPNLTVEEGK | |
| | SITLSCSVAGDPVPNMYWDVGNLVSKHMNETSHTQGSLRITNISSDDSGKQIS | |
| | CVAENLVGEDQDSVNLTVHFAPTITFLESPTSDHHWCIPFTVKGNPKPALQWF | |
| | YNGAILNESKYICTKIHVTNHTEYHGCLQLDNPTHMNNGDYTLIAKNEYGKDE | |
| | KQISAHFMGWPGIDDGANPNYPDVIYEDYGTAANDIGDTTNRSNEIPSTDVTD | |
| | KTGREHLSVYAVVVIASVVGFCLLVMLFLLKLARHSKFGMKGPASVISNDDDS | |
| | ASPLHHISNGSNTPSSSEGGPDAVIIGMTKIPVIENPQYFGITNSQLKPDTFV | |
| | QHIKRHNIVLKRELGEGAFGKVFLAECYNLCPEQDKILVAVKTLKDASDNARK | |
| | DFHREAELLTNLQHEHIVKFYGVCVEGDPLIMVFEYMKHGDLNKFLRAHGPDA | |
| | VLMAEGNPPTELTQSQMLHIAQQIAAGMVYLASQHFVHRDLATRNCLVGENLL | |
| | VKIGDFGMSRDVYSTDYYRVGGHTMLPIRWMPPESIMYRKFTTESDVWSLGVV | |
| | LWEIFTYGKQPWYQLSNNEVIECITQGRVLQRPRTCPQEVYELMLGCWQREPH | |
| | MRKNIKGIHTLLQNLAKASPVYLDILG | |
| 24 | CPTSCKCSASRIWCSDPSPGIVAFPRLEPNSVDPENITEIFIANQKRLEIINE | Trk-B |
| | DDVEAYVGLRNLTIVDSGLKFVAHKAFLKNSNLQHINFTRNKLTSLSRKHFRH | |
| | LDLSELILVGNPFTCSCDIMWIKTLQEAKSSPDTQDLYCLNESSKNIPLANLQ | |
| | IPNCGLPSANLAAPNLTVEEGKSITLSCSVAGDPVPNMYWDVGNLVSKHMNET | |
| | SHTQGSLRITNISSDDSGKQISCVAENLVGEDQDSVNLTVHFAPTITFLESPT | |
| | SDHHWCIPFTVKGNPKPALQWFYNGAILNESKYICTKIHVTNHTEYHGCLQLD | |
| | NPTHMNNGDYTLIAKNEYGKDEKQISAHFMGWPGIDDGANPNYPDVIYEDYGT | |
| | AANDIGDTTNRSNEIPSTDVTDKTGREHLSVYAVVVIASVVGFCLLVMLFLL | |
| 25 | CPTSCKCSASRIWCSDPSPGIVAFPRLEPNSVDPENITEIFIANQKRLEIINE | Trk-B |
| | DDVEAYVGLRNLTIVDSGLKFVAHKAFLKNSNLQHINFTRNKLTSLSRKHFRH | |
| | LDLSELILVGNPFTCSCDIMWIKTLQEAKSSPDTQDLYCLNESSKNIPLANLQ | |
| | IPNCGLPSANLAAPNLTVEEGKSITLSCSVAGDPVPNMYWDVGNLVSKHMNET | |
| | SHTQGSLRITNISSDDSGKQISCVAENLVGEDQDSVNLTVHFAPTITFLESPT | |
| | SDHHWCIPFTVKGNPKPALQWFYNGAILNESKYICTKIHVTNHTEYHGCLQLD | |
| | NPTHMNNGDYTLIAKNEYGKDEKQISAHFMGWPGIDDGANPNYPDVIYEDYGT | |
| | AANDIGDTTNRSNEIPSTDVTDKTGREH | |
| 26 | LSVYAVVVIASVVGFCLLVMLFLL | |
| 27 | MGAGATGRAMDGPRLLLLLLLGVSLGGAKEACPTGLYTHSGECCKACNLGEGV | p75^{NTR} |
| | AQPCGANQTVCEPCLDSVTFSDVVSATEPCKPCTECVGLQSMSAPCVEADDAV | |
| | CRCAYGYYQDETTGRCEACRVCEAGSGLVFSCQDKQNTVCEECPDGTYSDEAN | |
| | HVDPCLPCTVCEDTERQLRECTRWASAECEEIPGRWITRSTPPEGSDSTAPST | |
| | QEPEAPPEQDLIASTVAGVVTTVMGSSQPVVTRGTTDNLIPVYCSILAAVVVG | |
| | LVAYIAFKRWNSCKQNKQGANSRPVNQTPPPEGEKLHSDSGISVDSQSLHDQQ | |
| | PHTQTASGQALKGDGGLYSSLPPAKREEVEKLLNGSAGDTWRHLAGELGYQPE | |
| | HIDSFTHEACPVRALLASWATQDSATLDALLAALRRIQRADLVESLCSESTAT | |
| | SPV | |
| 28 | KEACPTGLYTHSGECCKACNLGEGVAQPCGANQTVCEPCLDSVTFSDVVSATE | |
| | PCKPCTECVGLQSMSAPCVEADDAVCRCAYGYYQDETTGRCEACRVCEAGSGL | |
| | VFSCQDKQNTVCEECPDGTYSDEANHVDPCLPCTVCEDTERQLRECTRWADAE | |
| | CEEIPGRWITRSTPPEGSDSTAPSTQEPEAPPEQDLIASTVAGVVTTVMGSSQ | |
| | PVVTRGTTDNLIPVYCSILAAVVVGLVAYIAF | |
| 29 | KEACPTGLYTHSGECCKACNLGEGVAQPCGANQTVCEPCLDSVTFSDVVSATE | |
| | PCKPCTECVGLQSMSAPCVEADDAVCRCAYGYYQDETTGRCEACRVCEAGSGL | |
| | VFSCQDKQNTVCEECPDGTYSDEANHVDPCLPCTVCEDTERQLRECTRWADAE | |
| | CEEIPGRWITRSTPPEGSDSTAPSTQEPEAPPEQDLIASTVAGVVTTVMGSSQ | |
| | PVVTRGTTDN | |
| 30 | LIPVYCSILAAVVGLVAYAIF | |
| 31 | APSPSLPDPKFESKAALLASRGSEELLCFTQRLEDLVCFWEEAASSGMDFNYS | Minimal EpoRm |
| | FSYQLEGESRKSCSLHQAPTVRGSVRFWCSLPTADTSSAVPLELQVTEASGSP | scaffold domain |
| | RYHRIIHINEVVLLDAPAGLLARRAEEGSHVVLRWLPPPGAPMTTHIRYEVDV | with F93A |
| | SAGNRAGGTQRVEVLEGRTECVLSNLRGGTRYTFAVRARMAEPSFSGFWSAWS | mutation |
| | EPASLLTASDLDPLILTLSLILVLISLLLTVLALLS | |
| 32 | MASRILWHEMWHEGLEEASRLYFGERNVKGMFEVLEPLHAMMERGPQTLKETS | FRB |
| | FNQAYGRDLMEAQEWCRKYMKSGNVKDLLQAWDLYYHVFRRISK | |
| 33 | MGVQVETISPGDGRTFPKRGQTCVVHYTGMLEDGKKFDSSRDRNKPFMFMLGK | FKBP |
| | QEVIRGWEEGVAQMSVGQRAKLTISPDYAYGATGHPGIIPPHATLVFDVELLK | |
| | LE | |
| 34 | VQLQESGGGLVQAGDSLKLSCEASGDSIGTYVIGWFRQAPGKERIYLATIGRN | VHH_{A52} |
| | LVGPSDFYTRYADSVKGRFAVSRDNAKNTVNLQMNSLKPEDTAVYYCAAKTTT | |
| | WGGNDPNNWNYWGQGTQVTV | |
| 35 | DIVMTQSPSSLSASVGDRVTITCRSSTGAVTTSNYASWVQEKPGKLFKGLIGG | SCFV_{αGCN4} |
| | TNNRAPGVPSRFSGSLIGDKATLTISSLQPEDFATYFCALWYSNHWVFGQGTK | |
| | VELKRGGGGSGGGGSGGGGSSGGGSEVKLLESGGGLVQPGGSLKLSCAVSGFS | |
| | LTDYGVNWVRQAPGRGLEWIGVIWGDGITDYNSALKDRFIISKDNGKNTVYLQ | |
| | MSKVRSDDTALYYCVTGLFDYWGQGTLVTVS | |
| 36 | DIVMTQTAPSVFVTPGESVSISCRSSKSLLHSNGNTYLYWFLQRPGQSPQLLI | SCF_{V5d3d11} |
| | YRMSNLASGVPDRFSGSGSGTDFTLRISRVEAEDVGVYYCMQHLEYPVTFGAG | |
| | TKVEIKRGGGGSGGGGSGGGGSGGGGSQVQLQQSGPELVKPGASVKISCKVSG | |
| | YAISSSWMNWVKQRPGHGLEWIGRIYPGDGDTKYNGKFKDKATLTVDKSSSTA | |
| | YMQLSSLTSVDSAVYFCARDGYRYYFDYWGQGTSVTVSS | |
| 37 | DIVLTQSPPSLAVSLGQRATISVRASESIDLYGFTFMHWYQQKPGQPPKILIY | ScF_{V5a5} |
| | RASNLESGIPARFSGSGRTDFTLTINPVEADDVATYYCQQTHEDPYTFGGGTK | |
| | LEIKRGGGGSGGGGSGGGGSGGGGSQVQLQQSGAELAKPGASVKMSCKTSGYS | |
| | FSSYMHWVKQRPGQGLEWIGYINPSTGYTENNQKFKDKVTLTADKSSNTAYMQ | |
| | LNSLTSEDSAVYYCARSGRLYFDVWGAGTTVTVSS | |
| 38 | DIVLTQSPASLAVSLGQRATISCKASQSVDFDGDSYMNWYQQKPGQPPKLLIF | SCF_{V8g8f5} |
| | AASNLASGIPARLSGSGSGTDFTLNIQPVEEEDAATYYCQQSNEDPYTFGGGT | |
| | KLEIKGGGGSGGGGSGGGGSGGGGSQVQLQQSGDDLVKPGASVKLSCKASGYT | |
| | FTTYYINWMRQRPGQGLEWIGRIAPASGTTYSSEMFKDKATLTVDTSSNTAYI | |
| | QLSSLSSEDSAVYFCARADYGFNSGEAMDYWGQGTSVTVSS | |
| 39 | QSELTQPPSASGTPGQRVTISCSGSSSNIGSNYVYWYQQLPGTAPKLLIYRNN | Nic12_{VL} |
| | QRPSGVPDRFSGSKSGTSASLAISGLRSEDEADYYCAAWDDSLSAWVFGGGTQ | |
| | LDILG | |
| 40 | QMQLLESGPGLVKPSETLSLTCTVSGGSIWGWIRQPPGKGLEWIGSIYSSGST | Nicl2_{VH} |
| | YYNPSLKSRVTTSVDTSKNQFSLRLSSVTAADTAVYYCVAWFGDLLSLKGVEL | |
| | WGQGTLVTVS | |
| 41 | NKRDLIKKHIWPNVPDPSKSHIAQWSPHTPPRHNFNSKDQMYSDGNFTDVSVV | IL-6RBᵢₙₜ |
| | EIEANDKKPFPEDLKSLDLFKKEKINTEGHSSGIGGSSCMSSSRPSISSSDEN | |
| | ESSQNTSSTVQYSTVVHSGYRHQVPSVQVFSRSESTQPLLDSEERPEDLQLVD | |
| | HVDGGDGILPRQQYFKQNCSQHESSPDISHFERSKQVSSVNEEDFVRLKQQIS | |
| | DHISQSCGSGQMKMFQEVSAADAFGPGTEGQVERFETVGMEAATDEGMPKSYL | |
| | PQTVRQGGYMPQ | |
| 42 | NKRDLIKKHIWPNVPDPSKSHIAQWSPHTPPRHNFNSKDQMYSDGNFTDVSVV | IL-6RBₘ with |
| | EIEANDKKPFPEDLKSLDLFKKEKINTEGHSSGIGGSSCMSSSRPSISSSDEN | Y759A mutation |
| | ESSQNTSSTVQASTVVHSGYRHQVPSVQVFSRSESTQPLLDSEERPEDLQLVD | |
| | HVDGGDGILPRQQYFKQNCSQHESSPDISHFERSKQVSSVNEEDFVRLKQQIS | |
| | DHISQSCGSGQMKMFQEVSAADAFGPGTEGQVERFETVGMEAATDEGMPKSYL | |
| | PQTVRQGGYMPQ | |
| 43 | LRTVKRANGGELKTGYLSIVMDPDELPLDEHCERLPYDASKWEFPRDRLKLGK | VEGFR2ᵢₙₜ |
| | PLGRGAFGQVIEADAFGIDKTATCRTVAVKMLKEGATHSEHRALMSELKILIH | |
| | GHHLNVVNLLGACTKPGGPLMVIVEFCKFGNLSTYLRSKRNEFVPYKTKGARF | |
| | RQGKDYVGAIPVDLKRRLDSITSSQSSASSGFVEEKSLSDVEEEEAPEDLYKD | |
| | FLTLEHLICYSFQVAKGMEFLASRKCIHRDLAARNILLSEKNVVKICDFGLAR | |
| | IYKDPDYVRKGDARLPLKWMAPETIFDRVYTIQSDVWSFGVLLWEIFSLGASP | |
| | YPGVKIDEEFCRRLKEGTRMRAPDYTTPEMYQTMLDCWHGEPSQRPTFSELVE | |
| | HLGNLLQANAQQDGKDYIVLPISETLSMEEDSGLSLPTSPVSCMEEEEVCDPK | |
| | FHYDNTAGISQYLQNSKRKSRPVSVKTFEDIPLEEPEVKVIPDDNQTDSGMVL | |
| | ASEELKTLEDRTKLSPSFGGMVPSKSRESVASEGSNQTSGYQSGYHSDDTTVY | |
| | SSEEAELLKLIEIGVQTGSTAQILQPDSGTTLSSPPV | |
| 44 | MKSGTKKSDFHSQMAVHKLAKSIPLRRQVTVSADSSASMNSGVLLVRPSRLSS | FGFR1ᵢₙₜ |
| | SGTPMLAGVSEYELPEDPRWELPRDRLVLGKPLGEGCFGQVVLAEAIGLDKDK | |
| | PNRVTKVAVKMLKSDATEKDLSDLISEMEMMKMIGKHKNIINLLGACTQDGPL | |
| | YVIVEYASKGNLREYLQARRPPGLEYCYNPSHNPEEQLSSKDLVSCAYQVARG | |
| | MEYLASKKCIHRDLAARNVLVTEDNVMKIADFGLARDIHHIDYYKKTTNGRLP | |
| | VKWMAPEALFDRIYTHQSDVWSFGVLLWEIFTLGGSPYPGVPVEELFKLLKEG | |
| | HRMDKPSNCTNELYMMMRDCWHAVPSQRPTFKQLVEDLDRIVALTSNQEYLDL | |
| | SIPLDQYSPSFPDTRSSTCSSGEDSVFSHEPLPEEPCLPRHPTQLANSGLKRR | |

For reasons of completeness, various aspects of the disclosure are set out in the following numbered embodiments:
1. A chimeric ligand receptor comprising a receptor subunit, wherein the receptor subunit comprises a scaffold domain;
   wherein the scaffold domain comprises an extracellular domain and a transmembrane domain of a receptor selected from the group consisting of a leptin receptor (Ob-R), a growth hormone receptor (GHR), an epidermal growth factor receptor (EGFR), Trk-B, and p75^{NTR};
   wherein the extracellular domain is operably linked to a ligand binding domain;
   wherein the transmembrane domain is operably linked to an intracellular signaling domain;
   wherein the receptor subunit multimerizes via its scaffold domain in the presence of one or more additional receptor subunits; and
   wherein the multimerized receptor subunits undergo a conformational reorganization upon ligand binding to the chimeric ligand receptor.
2. The chimeric ligand receptor of claim 1, further comprising one or more additional receptor subunits, wherein each additional receptor subunit comprises a scaffold domain;
   wherein the scaffold domain of each additional receptor subunit comprises an extracellular domain and a transmembrane domain of a receptor selected from the group consisting of a leptin receptor (Ob-R), a growth hormone receptor (GHR), an epidermal growth factor receptor (EGFR), Trk-B, and p75^{NTR};
   wherein the extracellular domain of each additional receptor subunit is operably linked to a ligand binding domain; and
   wherein the transmembrane domain of each additional receptor subunit is operably linked to an intracellular signaling domain.
3. The chimeric ligand receptor of claim 2, wherein the receptor subunits multimerize via their scaffold domains to form the chimeric ligand receptor.
4. The chimeric ligand receptor of claim 3, wherein the multimerized receptor subunits undergo a conformational reorganization upon ligand binding to the chimeric receptor.
5. The chimeric ligand receptor of any one of claims 1-4, wherein the chimeric ligand binding domains of each receptor subunit bind the same ligand.
6. A chimeric ligand receptor comprising two or more receptor subunits,
   wherein each receptor subunit comprises a scaffold domain;
   wherein the receptor subunits multimerize via their scaffold domains to form the chimeric ligand receptor;
   wherein each scaffold domain comprises an extracellular domain and a transmembrane domain of a receptor selected from the group consisting of a leptin receptor (Ob-R), a growth hormone receptor (GHR), an epidermal growth factor receptor (EGFR), Trk-B, and p75^{NTR};
   wherein the extracellular domain is operably linked to a ligand binding domain, wherein the ligand binding domains of each receptor subunit bind the same ligand;
   wherein the transmembrane domain is operably linked to an intracellular signaling domain; and
   wherein binding of the ligand induces a conformational reorganization of the multimerized receptor subunits.
7. The chimeric ligand receptor of any one of claims 3-6, wherein multimerization of the receptor subunits occurs prior to ligand binding.
8. The chimeric ligand receptor of any one of claims 3-7, wherein the multimerized receptor subunits comprise a dimer, a trimer, tetramer, pentamer, or hexamer.
9. The chimeric ligand receptor of any one of claims 3-8, wherein the multimerized receptor subunits comprise a dimer.
10. The chimeric ligand receptor of any one of claims 4-9, wherein the conformational reorganization comprises a rotation of each scaffold domain around its own axis.
11. The chimeric ligand receptor of any one of claims 4-10, wherein the conformational reorganization activates the intracellular signaling domains of each receptor subunit.
12. The chimeric ligand receptor of any one of claims 4-10, wherein the conformational reorganization inhibits the intracellular signaling domains of each receptor subunit.
13. The chimeric ligand receptor of any one of claims 1-12, wherein the scaffold domain is inert to its native ligand.
14. The chimeric ligand receptor of any one of claims 1-13, wherein the scaffold domain comprises the extracellular domain and transmembrane domain of a leptin receptor (Ob-R).
15. The chimeric ligand receptor of claim 14, wherein the scaffold domain is inert to leptin.
16. The chimeric ligand receptor of any one of claims 1-15, wherein the scaffold domain comprises an extracellular domain and transmembrane domain comprising an amino acid sequence having 90% or greater sequence identity to SEQ ID NO: 12.
17. The chimeric ligand receptor of any one of claims 1-13, wherein the scaffold domain comprises the extracellular domain and transmembrane domain of a growth hormone receptor (GHR).
18. The chimeric ligand receptor of claim 17, wherein the scaffold domain is inert to growth hormone.
19. The chimeric ligand receptor of claim 17 or claim 18, wherein the scaffold domain comprises an extracellular domain and transmembrane domain comprising an amino acid sequence having 90% or greater sequence identity to SEQ ID NO: 16.
20. The chimeric ligand receptor of any one of claims 1-13, wherein the scaffold domain comprises the extracellular domain and transmembrane domain of an epidermal growth factor receptor (EGFR).
21. The chimeric ligand receptor of claim 20, wherein the scaffold domain is inert to epidermal growth factor (EGF).
22. The chimeric ligand receptor of claim 20 or claim 21, wherein the scaffold domain comprises an extracellular domain and transmembrane domain comprising an amino acid sequence having 90% or greater sequence identity to SEQ ID NO: 20.
23. The chimeric ligand receptor of any one of claims 1-13, wherein the scaffold domain comprises the extracellular domain and transmembrane domain of Trk-B.
24. The chimeric ligand receptor of any one of claims 1-13, wherein the scaffold domain comprises the extracellular domain and transmembrane domain of p75^{NTR}.
25. The chimeric ligand receptor of claim 23 or claim 24, wherein the scaffold domain is inert to brain-derived neurotrophic factor (BDNF).
26. The chimeric ligand receptor of claim 23 or claim 25, wherein the scaffold domain comprises an extracellular domain and transmembrane domain comprising an amino acid sequence having 90% or greater sequence identity to SEQ ID NO: 24.
27. The chimeric ligand receptor of claim 24 or claim 25, wherein the scaffold domain comprises an extracellular domain and transmembrane domain comprising an amino acid sequence having 90% or greater sequence identity to SEQ ID NO: 28.
28. The chimeric ligand receptor of any one of claims 1-27, wherein the extracellular domain, the transmembrane domain, or both the extracellular domain and transmembrane domain of the scaffold domain comprise one or more modifications.
29. The chimeric ligand receptor of claim 1-28, wherein the extracellular domain comprises one or more amino acid substitutions.
30. The chimeric ligand receptor of any one of claims 1-29, wherein one or more additional amino acid residues are inserted adjacent to the transmembrane domain.
31. The chimeric ligand receptor of any one of claims 1-29, wherein one or more additional amino acid residues are inserted within the transmembrane domain.
32. The chimeric ligand receptor of claim 30 or claim 31, wherein the one or more additional amino acid residues are alanine residues.
33. The chimeric ligand receptor of claim 32, wherein the transmembrane domain further comprises one, two, three, or four additional alanine residues.
34. The chimeric ligand receptor of any one of claims 30-33, wherein the one or more additional amino acid residues are inserted C-terminal to the transmembrane domain.
35. The chimeric ligand receptor of any one of claims 1-34, wherein the ligand binding domain is linked to the extracellular domain through an extracellular linker region.
36. The chimeric ligand receptor of claim 35, wherein the extracellular linker region comprises one or more amino acid residues, optionally wherein the one or more amino acid residues comprise amino acids residues Serine-Glycine-Glutamic acid-Phenylalanine.
37. The chimeric ligand receptor of any one of claims 1-36, wherein the ligand binding domain does not bind a native ligand of the scaffold domain.
38. The chimeric ligand receptor of any one of claims 1-36, wherein the ligand binding domain does not bind leptin.
39. The chimeric ligand receptor of any one of claims 1-36, wherein the ligand binding domain does not bind growth hormone.
40. The chimeric ligand receptor of any one of claims 1-36, wherein the ligand binding domain does not bind epidermal growth factor (EGF).
41. The chimeric ligand receptor of any one of claims 1-36, wherein the ligand binding domain does not bind brain-derived neurotrophic factor (BDNF).
42. The chimeric ligand receptor of any one of claims 1-41, wherein the ligand binding domain binds to a ligand selected from the group consisting of a protein complex, a protein, a peptide, a nucleic acid, a small molecule, and a chemical agent.
43. The chimeric ligand receptor of claim 42, wherein the ligand is selected from the group consisting of an antigen, a cytokine, a survival factor, a chemokine, a hormone, a transmitter, a growth factor, extracellular matrix, and a death factor.
44. The chimeric ligand receptor of any one of claims 1-43, wherein the ligand binding domain binds to a ligand displayed on a surface or expressed on a cell surface.
45. The chimeric ligand receptor of claim 44, wherein the the ligand expressed on a cell surface is a tumor-associated antigen.
46. The chimeric ligand receptor of claim, wherein the tumor-associated antigen is selected from the group consisting of MS4A3, VSTM1, LAT2, MLC1, CD131, GAPT, PRAM1, SLC22A16, SLC17A9, SPNS3, FLT3, CD33, CLEC12A, ADGRE2, IL3RA, CD117, CD93, IL1RAP, CD244, CCR1, LILRB2, PIEZO1, CD38, EMB, MYADM, LILRA2, CD300LF, CD70, 5T4, ADAM9, AFP, AXL, B7-H3, B7-H4, B7-H6, C4.4, CA6, Cadherin 3, Cadherin 6, CCR4, CD123, CD133, CD138, CD142, CD166, CD25, CD30, CD352, CD37, CD38, CD44, CD56, CD66e, CD71, CD74, CD79b, CD80, CEA, CEACAM5, Claudin18.2, cMet, CSPG4, CTLA, DLK1, DLL3, DR5, EGFR, GCC, ENPP3, EpCAM, EphA2, Ephrin A4, ETBR, FGFR2, FGFR3, FRalpha, FRb, GCC, GD2, GFRa4, gpA33, GPC3, gpNBM, GPRC5, HER2, IL-13R, IL-13Ra2, IL-8, IL-15, IL1RAP, Integrin aV, KIT, L1CAM, LAMP1, Lewis Y, LeY, LIV-1, LRRC, LY6E, MCSP, Mesothelin, MUC1, MUC16, MUC1C, NaPi2B, Nectin 4, NKG2D, NOTCH3, NY ESO 1, Ovarin, P-cadherin, pan-Erb2, PSCA, PSMA, PTK7, ROR1, S Aures, SCT, SLAMF7, SLITRK6, SSTR2, STEAP1, Survivin, TDGF1, TIM1, TROP2, and WT1.
47. The chimeric ligand receptor of any one of claims 1-43, wherein the ligand binding domain binds to a soluble ligand.
48. The chimeric ligand receptor of claim 47, wherein the soluble ligand is a secreted cytokine.
49. The chimeric ligand receptor of claim 48, wherein the secreted cytokine is an immunosuppressive cytokine.
50. The chimeric ligand receptor of claim 48, wherein the secreted cytokine is selected from the group consisting of TGFbeta, IL-10, IL-6, IL-23, IL-8, CCL2, CXCL12, CXCL8, CXCL1, CXCL2, CXCL3, CXCL8, CXCL13, CCL5, CCL17, and CCL22.
51. The chimeric ligand receptor of any one of claims 1-50, wherein the ligand binding domain binds to an antigen.
52. The chimeric ligand receptor of claim 51, wherein the ligand binding domain comprises an antibody, or antigen-binding fragment thereof.
53. The chimeric ligand receptor of claim 52, wherein the ligand binding domain comprises a single chain variable fragment (scFv), or a single-domain antibody (sdAb).
54. The chimeric ligand receptor of any one of claims 2-48, wherein each of the ligand binding domains comprises a single chain variable fragment (scFv), optionally wherein each scFv specifically binds to a distinct epitope of the antigen.
55. The chimeric ligand receptor of any one of claims 2-54, wherein the ligand binding domains of each receptor subunit are distinct from one another.
56. The chimeric ligand receptor of claim 55, wherein the chimeric ligand receptor comprises two ligand binding domains, and wherein one ligand binding domain comprises an immunoglobulin heavy chain variable domain (V_{H}) and the second ligand binding domain comprises an immunoglobulin light chain variable domain (V_{L}).
57. The chimeric ligand receptor of any one of claims 2-54, wherein the ligand binding domains of each receptor subunit are the same as one another.
58. The chimeric ligand receptor of claim 52, wherein the antibody, or antigen-binding fragment thereof, is a single-domain VHH camelid antibody.
59. The chimeric ligand receptor of any one of claims 1-58, wherein the intracellular signaling domain is inert to native ligand binding of the scaffold domain.
60. The chimeric ligand receptor of any one of claims 1-59, wherein the intracellular signaling domain does not comprise an endogenous intracellular signaling domain of the scaffold domain.
61. The chimeric ligand receptor of any one of claims 1-59, wherein the intracellular signaling domain does not comprise an endogenous leptin receptor (Ob-R) intracellular signaling domain.
62. The chimeric ligand receptor of any one of claims 1-59, wherein the intracellular signaling domain does not comprise an endogenous growth hormone receptor (GHR) intracellular signaling domain.
63. The chimeric ligand receptor of any one of claims 1-59, wherein the intracellular signaling domain does not comprise an endogenous epidermal growth factor receptor (EGFR) intracellular signaling domain.
64. The chimeric ligand receptor of any one of claims 1-59, wherein the intracellular signaling domain does not comprise an endogenous Trk-B intracellular signaling domain.
65. The chimeric ligand receptor of any one of claims 1-59, wherein the intracellular signaling domain does not comprise an endogenous p75^{NTR} intracellular signaling domain.
66. The chimeric ligand receptor of any one of claims 1-65, wherein the intracellular signaling domain induces downstream signaling via a JAK/STAT (Janus kinase/signal transducer and activator of transcription) signaling pathway, a MAPK (mitogen-activated protein kinase) signaling pathway, a PLCG (phospholipase C gamma) signaling pathway, or a PI3K/Akt (phosphatidylinositol 3-kinase/protein kinase B) signaling pathway.
67. The chimeric ligand receptor of any one of claims 1-66, wherein the intracellular signaling domain is selected from the group consisting of an intracellular signal transduction domain of IL-6RB (interleukin 6 receptor B), an intracellular signal transduction domain of FGFR1 (fibroblast growth factor receptor 1), and an intracellular signal transduction domain of VEGFR2 (vascular endothelial growth factor receptor 2).
68. The chimeric ligand receptor of any one of claims 1-65, wherein the intracellular signaling domain is an intracellular signal transduction domain of IL-6RB and induces downstream signaling via the JAK/STAT signaling pathway.
69. The chimeric ligand receptor of any one of claims 1-65, wherein the intracellular signaling domain is an intracellular signal transduction domain of FGFR1 and induces downstream signaling via the MAPK signaling pathway.
70. The chimeric ligand receptor of any one of claims 1-65, wherein the intracellular signaling domain is an intracellular signal transduction domain of VEGFR2 and induces downstream signaling via the PLCG signaling pathway.
71. The chimeric ligand receptor of any one of claims 1-65, wherein the intracellular signaling domain is an intracellular signal transduction domain of VEGFR2 and induces downstream signaling via the PI3K/Akt signaling pathways.
72. The chimeric ligand receptor of any one of claims 1-65, wherein the intracellular signaling domain is an intracellular signal transduction domain derived from an IL-2 signaling pathway, an IL-7 signaling pathway, or an IL-15 signaling pathway.
73. The chimeric ligand receptor of any one of claims 1-72, wherein the intracellular signaling domain comprises one or more modifications that modulate signaling activity of the intracellular signaling domain, optionally wherein the one or more modifications are one or more amino acid substitutions.
74. An isolated polynucleotide or a set of isolated polynucleotides encoding the chimeric ligand receptor of any one of claims 1-73.
75. The isolated polynucleotide or set of isolated polynucleotides of claim 74, comprising the cDNA of the chimeric ligand receptor of any one of claims 1-73.
76. An isolated polynucleotide or a set of isolated polynucleotides comprising a nucleic acid sequence encoding a chimeric ligand receptor subunit, wherein the receptor subunit comprises a scaffold domain capable of multimerizing the receptor subunit in the presence of one or more additional receptor subunits and wherein the receptor subunit is capable of undergoing a conformational reorganization induced by ligand binding when multimerized;
   wherein the scaffold domain comprises an extracellular domain and a transmembrane domain of a receptor selected from the group consisting of a leptin receptor (Ob-R), a growth hormone receptor (GHR), an epidermal growth factor receptor (EGFR), Trk-B, and p75^{NTR};
   wherein the extracellular domain is operably linked to a ligand binding domain; and
   wherein the transmembrane domain is operably linked to an intracellular signaling domain.
77. The isolated polynucleotide or set of isolated polynucleotides of claim 76, further comprising one or more nucleic acid sequences, wherein each additional nucleic acid sequence encoding an additional chimeric ligand receptor subunit;
   wherein each additional receptor subunit comprises a scaffold domain;
   wherein the scaffold domain of each additional receptor subunit comprises an extracellular domain and a transmembrane domain of a receptor selected from the group consisting of a leptin receptor (Ob-R), a growth hormone receptor (GHR), an epidermal growth factor receptor (EGFR), Trk-B, and p75^{NTR};
   wherein the extracellular domain of each additional receptor subunit is operably linked to a ligand binding domain; and
   wherein the transmembrane domain of each additional receptor subunit is operably linked to an intracellular signaling domain.
78. The isolated polynucleotide or set of isolated polynucleotides of claim 76 or claim 77, wherein the receptor subunits multimerize via their scaffold domains to form the chimeric ligand receptor.
79. The isolated polynucleotide or set of isolated polynucleotides of any one of claims 76-78, wherein the multimerized receptor subunits undergo a conformational reorganization upon ligand binding to the chimeric receptor.
80. The isolated polynucleotide or set of isolated polynucleotides of any one of claims 76-79, wherein the chimeric ligand binding domains of each receptor subunit bind the same ligand.
81. An isolated polynucleotide or a set of isolated polynucleotides comprising two or more nucleic acid sequences, wherein each nucleic acid sequence encoding a chimeric ligand receptor subunit;
   wherein each receptor subunit comprises a scaffold domain;
   wherein the receptor subunits multimerize via their scaffold domains to form the chimeric ligand receptor;
   wherein each scaffold domain comprises an extracellular domain and a transmembrane domain of a receptor selected from the group consisting of a leptin receptor (Ob-R), a growth hormone receptor (GHR), an epidermal growth factor receptor (EGFR), Trk-B, and p75^{NTR}:
   wherein the extracellular domain is operably linked to a ligand binding domain, wherein the ligand binding domains of each receptor subunit bind the same ligand;
   wherein the transmembrane domain is operably linked to an intracellular signaling domain; and
   wherein binding of the ligand induces a conformational reorganization of the multimerized receptor subunits.
82. The isolated polynucleotide or set of isolated polynucleotides of any one of claims 78-81, wherein multimerization of the receptor subunits occurs prior to ligand binding.
83. The isolated polynucleotide or set of isolated polynucleotides of any one of claims 78-82, wherein the multimerized receptor subunits comprise a dimer, a trimer, tetramer, pentamer, or hexamer.
84. The isolated polynucleotide or set of isolated polynucleotides of any one of claims 78-83, wherein the multimerized receptor subunits comprise a dimer.
85. The isolated polynucleotide or set of isolated polynucleotides of any one of claims 79-84, wherein the conformational reorganization comprises a rotation of each scaffold domain around its own axis.
86. The isolated polynucleotide or set of isolated polynucleotides of any one of claims 79-85, wherein the conformational reorganization activates the intracellular signaling domains of each receptor subunit.
87. The isolated polynucleotide or set of isolated polynucleotides of any one of claims 79-85, wherein the conformational reorganization inhibits the intracellular signaling domains of each receptor subunit.
88. The isolated polynucleotide or set of isolated polynucleotides of any one of claims 76-87, wherein the scaffold domain is inert to its native ligand.
89. The isolated polynucleotide or set of isolated polynucleotides of any one of claims 76-88, wherein the scaffold domain comprises the extracellular domain and transmembrane domain of a leptin receptor (Ob-R).
90. The isolated polynucleotide or set of isolated polynucleotides of claim 89, wherein the scaffold domain is inert to leptin.
91. The isolated polynucleotide or set of isolated polynucleotides of claim 89 or claim 90, wherein the scaffold domain comprises an extracellular domain and transmembrane domain comprising an amino acid sequence having 90% or greater sequence identity to SEQ ID NO: 12.
92. The isolated polynucleotide or set of isolated polynucleotides of any one of claims 76-88, wherein the scaffold domain comprises the extracellular domain and transmembrane domain of a growth hormone receptor (GHR).
93. The isolated polynucleotide or set of isolated polynucleotides of claim 92, wherein the scaffold domain is inert to growth hormone.
94. The isolated polynucleotide or set of isolated polynucleotides of claim 92 or claim 93, wherein the scaffold domain comprises an extracellular domain and transmembrane domain comprising an amino acid sequence having 90% or greater sequence identity to SEQ ID NO: 16.
95. The isolated polynucleotide or set of isolated polynucleotides of any one of claims 76-88, wherein the scaffold domain comprises the extracellular domain and transmembrane domain of an epidermal growth factor receptor (EGFR).
96. The isolated polynucleotide or set of isolated polynucleotides of claim 95, wherein the scaffold domain is inert to epidermal growth factor (EGF).
97. The isolated polynucleotide or set of isolated polynucleotides of claim 95 or claim 96, wherein the scaffold domain comprises an extracellular domain and transmembrane domain comprising an amino acid sequence having 90% or greater sequence identity to SEQ ID NO: 20.
98. The isolated polynucleotide or set of isolated polynucleotides of any one of claims 76-88, wherein the scaffold domain comprises the extracellular domain and transmembrane domain of Trk-B.
99. The isolated polynucleotide or set of isolated polynucleotides of any one of claims 76-88, wherein the scaffold domain comprises the extracellular domain and transmembrane domain of p75^{NTR}.
100. The isolated polynucleotide or set of isolated polynucleotides of claim 98 or claim 99, wherein the scaffold domain is inert to brain-derived neurotrophic factor (BDNF).
101. The isolated polynucleotide or set of isolated polynucleotides of claim 98 or claim 100, wherein the scaffold domain comprises an extracellular domain and transmembrane domain comprising an amino acid sequence having 90% or greater sequence identity to SEQ ID NO: 24.
102. The isolated polynucleotide or set of isolated polynucleotides of claim 99 or claim 100, wherein the scaffold domain comprises an extracellular domain and transmembrane domain comprising an amino acid sequence having 90% or greater sequence identity to SEQ ID NO: 28.
103. The isolated polynucleotide or set of isolated polynucleotides of any one of claims 76-102, wherein the extracellular domain, the transmembrane domain, or both the extracellular domain and transmembrane domain of the scaffold domain comprise one or more modifications.
104. The isolated polynucleotide or set of isolated polynucleotides of any one of claims 76-103, wherein the extracellular domain comprises one or more amino acid substitutions.
105. The isolated polynucleotide or set of isolated polynucleotides of any one of claims 76-104, wherein one or more additional amino acid residues are inserted adjacent to the transmembrane domain.
106. The isolated polynucleotide or set of isolated polynucleotides of any one of claims 76-104, wherein one or more additional amino acid residues are inserted within the transmembrane domain.
107. The isolated polynucleotide or set of isolated polynucleotides of claim 105 or claim 100, wherein the one or more additional amino acid residues are alanine residues.
108. The isolated polynucleotide or set of isolated polynucleotides of claim 107, wherein the transmembrane domain further comprises one, two, three, or four additional alanine residues.
109. The isolated polynucleotide or set of isolated polynucleotides of any one of claims 105-108, wherein the one or more additional amino acid residues are inserted C-terminal to the transmembrane domain.
110. The isolated polynucleotide or set of isolated polynucleotides of any one of claims 76-109, wherein the ligand binding domain is linked to the extracellular domain through an extracellular linker region.
111. The isolated polynucleotide or set of isolated polynucleotides of claim 110, wherein the extracellular linker region comprises one or more amino acid residues, optionally wherein the one or more amino acid residues comprise amino acids residues Serine-Glycine-Glutamic acid-Phenylalanine.
112. The isolated polynucleotide or set of isolated polynucleotides of any one of claims 76-111, wherein the ligand binding domain does not bind a native ligand of the scaffold domain.
113. The isolated polynucleotide or set of isolated polynucleotides of any one of claims 76-111, wherein the ligand binding domain does not bind leptin.
114. The isolated polynucleotide or set of isolated polynucleotides of any one of claims 76-111, wherein the ligand binding domain does not bind growth hormone.
115. The isolated polynucleotide or set of isolated polynucleotides of any one of claims 76-111, wherein the ligand binding domain does not bind epidermal growth factor (EGF).
116. The isolated polynucleotide or set of isolated polynucleotides of any one of claims 76-111, wherein the ligand binding domain does not bind brain-derived neurotrophic factor (BDNF).
117. The isolated polynucleotide or set of isolated polynucleotides of any one of claims 76-116, wherein the ligand binding domain binds to a ligand selected from the group consisting of a protein complex, a protein, a peptide, a nucleic acid, a small molecule, and a chemical agent.
118. The isolated polynucleotide or set of isolated polynucleotides of claim 117, wherein the ligand is selected from the group consisting of an antigen, a cytokine, a survival factor, a chemokine, a hormone, a transmitter, a growth factor, extracellular matrix, and a death factor.
119. The isolated polynucleotide or set of isolated polynucleotides of any one of claims 76-118, wherein the ligand binding domain binds to a ligand displayed on a surface or expressed on a cell surface.
120. The isolated polynucleotide or set of isolated polynucleotides of claim 119, wherein the the ligand expressed on a cell surface is a tumor-associated antigen.
121. The isolated polynucleotide or set of isolated polynucleotides of claim 120, wherein the tumor-associated antigen is selected from the group consisting of MS4A3, VSTM1, LAT2, MLC1, CD131, GAPT, PRAM1, SLC22A16, SLC17A9, SPNS3, FLT3, CD33, CLEC12A, ADGRE2, IL3RA, CD117, CD93, IL1RAP, CD244, CCR1, LILRB2, PIEZO1, CD38, EMB, MYADM, LILRA2, CD300LF, CD70, 5T4, ADAM9, AFP, AXL, B7-H3, B7-H4, B7-H6, C4.4, CA6, Cadherin 3, Cadherin 6, CCR4, CD123, CD133, CD138, CD142, CD166, CD25, CD30, CD352, CD37, CD38, CD44, CD56, CD66e, CD71, CD74, CD79b, CD80, CEA, CEACAM5, Claudin18.2, cMet, CSPG4, CTLA, DLK1, DLL3, DR5, EGFR, ENPP3, EpCAM, EphA2, Ephrin A4, ETBR, FGFR2, FGFR3, FRalpha, FRb, GCC, GD2, GFRa4, gpA33, GPC3, gpNBM, GPRC5, HER2, IL-13R, IL-13Ra2, IL-8, IL-15, IL1RAP, Integrin aV, KIT, L1CAM, LAMP1, Lewis Y, LeY, LIV-1, LRRC, LY6E, MCSP, Mesothelin, MUC1, MUC16, MUC1C, NaPi2B, Nectin 4, NKG2D, NOTCH3, NY ESO 1, Ovarin, P-cadherin, pan-Erb2, PSCA, PSMA, PTK7, ROR1, S Aures, SCT, SLAMF7, SLITRK6, SSTR2, STEAP1, Survivin, TDGF1, TIM1, TROP2, and WT1.
122. The isolated polynucleotide or set of isolated polynucleotides of any one of claims 76-118, wherein the ligand binding domain binds to a soluble ligand.
123. The isolated polynucleotide or set of isolated polynucleotides of claim 122, wherein the soluble ligand is a secreted cytokine.
124. The isolated polynucleotide or set of isolated polynucleotides of claim 123, wherein the secreted cytokine is an immunosuppressive cytokine.
125. The isolated polynucleotide or set of isolated polynucleotides of claim 123, wherein the secreted cytokine is selected from the group consisting of TGFbeta, IL-10, IL-6, IL-23, IL-8, CCL2, CXCL12, CXCL8, CXCL1, CXCL2, CXCL3, CXCL8, CXCL13, CCL5, CCL17, and CCL22.
126. The isolated polynucleotide or set of isolated polynucleotides of any one of claims 76-125, wherein the ligand binding domain binds to an antigen.
127. The isolated polynucleotide or set of isolated polynucleotides of claim 126, wherein the ligand binding domain comprises an antibody, or antigen-binding fragment thereof.
128. The isolated polynucleotide or set of isolated polynucleotides of claim 127, wherein the ligand binding domain comprises a single chain variable fragment (scFv), or a single-domain antibody (sdAb).
129. The isolated polynucleotide or set of isolated polynucleotides of any one of claims 77-128, wherein each of the ligand binding domains comprises a single chain variable fragment (scFv), optionally wherein each scFv specifically binds to a distinct epitope of the antigen.
130. The isolated polynucleotide or set of isolated polynucleotides of any one of claims 77-129, wherein the ligand binding domains of each receptor subunit are distinct from one another.
131. The isolated polynucleotide or set of isolated polynucleotides of claim 130, wherein the chimeric ligand receptor comprises two ligand binding domains, and wherein one ligand binding domain comprises an immunoglobulin heavy chain variable domain (V_{H}) and the second ligand binding domain comprises an immunoglobulin light chain variable domain (V_{L}).
132. The isolated polynucleotide or set of isolated polynucleotides of any one of claims 77-129, wherein the ligand binding domains of each receptor subunit are the same as one another.
133. The isolated polynucleotide or set of isolated polynucleotides of claim 127, wherein the antibody, or antigen-binding fragment thereof, is a single-domain VHH camelid antibody.
134. The isolated polynucleotide or set of isolated polynucleotides of any one of claims 76-133, wherein the intracellular signaling domain is inert to native ligand binding of the scaffold domain.
135. The isolated polynucleotide or set of isolated polynucleotides of any one of claims 76-134, wherein the intracellular signaling domain does not comprise an endogenous intracellular signaling domain of the scaffold domain.
136. The isolated polynucleotide or set of isolated polynucleotides of any one of claims 76-134, wherein the intracellular signaling domain does not comprise an endogenous leptin receptor (Ob-R) intracellular signaling domain.
137. The isolated polynucleotide or set of isolated polynucleotides of any one of claims 76-134, wherein the intracellular signaling domain does not comprise an endogenous growth hormone receptor (GHR) intracellular signaling domain.
138. The isolated polynucleotide or set of isolated polynucleotides of any one of claims 76-134, wherein the intracellular signaling domain does not comprise an endogenous epidermal growth factor receptor (EGFR) intracellular signaling domain.
139. The isolated polynucleotide or set of isolated polynucleotides of any one of claims 76-134, wherein the intracellular signaling domain does not comprise an endogenous Trk-B intracellular signaling domain.
140. The isolated polynucleotide or set of isolated polynucleotides of any one of claims 76-134, wherein the intracellular signaling domain does not comprise an endogenous p75^{NTR} intracellular signaling domain.
141. The isolated polynucleotide or set of isolated polynucleotides of any one of claims 76-140, wherein the intracellular signaling domain induces downstream signaling via a JAK/STAT (Janus kinase/signal transducer and activator of transcription) signaling pathway, a MAPK (mitogen-activated protein kinase) signaling pathway, a PLCG (phospholipase C gamma) signaling pathway, or a PI3K/Akt (phosphatidylinositol 3-kinase/protein kinase B) signaling pathway.
142. The isolated polynucleotide or set of isolated polynucleotides of any one of claims 76-140, wherein the intracellular signaling domain is selected from the group consisting of an intracellular signal transduction domain of IL-6RB (interleukin 6 receptor B), an intracellular signal transduction domain of FGFR1 (fibroblast growth factor receptor 1), and an intracellular signal transduction domain of VEGFR2 (vascular endothelial growth factor receptor 2).
143. The isolated polynucleotide or set of isolated polynucleotides of any one of claims 76-140, wherein the intracellular signaling domain is an intracellular signal transduction domain of IL-6RB and induces downstream signaling via the JAK/STAT signaling pathway.
144. The isolated polynucleotide or set of isolated polynucleotides of any one of claims 76-140, wherein the intracellular signaling domain is an intracellular signal transduction domain of FGFR1 and induces downstream signaling via the MAPK signaling pathway.
145. The isolated polynucleotide or set of isolated polynucleotides of any one of claims 76-140, wherein the intracellular signaling domain is an intracellular signal transduction domain of VEGFR2 and induces downstream signaling via the PLCG signaling pathway.
146. The isolated polynucleotide or set of isolated polynucleotides of any one of claims 76-140, wherein the intracellular signaling domain is an intracellular signal transduction domain of VEGFR2 and induces downstream signaling via the PI3K/Akt signaling pathways.
147. The isolated polynucleotide or set of isolated polynucleotides of any one of claims 76-140, wherein the intracellular signaling domain is an intracellular signal transduction domain derived from an IL-2 signaling pathway, an IL-7 signaling pathway, or an IL-15 signaling pathway.
148. The isolated polynucleotide or set of isolated polynucleotides of any one of claims 76-147, wherein the intracellular signaling domain comprises one or more modifications that modulate signaling activity of the intracellular signaling domain, optionally wherein the one or more modifications are one or more amino acid substitutions.
149. A vector or a set of vectors comprising the polynucleotide or set of polynucleotides of any one of claims 74-148.
150. A genetically engineered cell comprising the polynucleotide or set of polynucleotides of any one of claims 74-148 or the vector or set of vectors of claim 149.
151. A genetically engineered cell expressing the chimeric ligand receptor of any one of claims 1-73.
152. The genetically engineered cell of claim 150 or claim 151, wherein the cell further comprises an engineered transgene, wherein the transgene comprises a synthetic promoter operably linked to a polynucleotide comprising a nucleic acid sequence encoding a target product.
153. The genetically engineered cell of claim 152, wherein the synthetic promoter is responsive to intracellular signaling from the chimeric ligand receptor.
154. The genetically engineered cell of claim 152 or claim 153, wherein the target product is selected from the group consisting of a therapeutic molecule, a prophylactic molecule, and a diagnostic molecule.
155. The genetically engineered cell of any one of claims 152-154, wherein the target product is a chimeric antigen receptor (CAR).
156. The genetically engineered cell of any one of claims 152-154, wherein the target product is a proinflammatory cytokine.
157. The genetically engineered cell of claim 156, wherein the proinflammatory cytokine is selected from the group consisting of IL-2, IL-7, IL-12, IL-15, IL-18, and IL-21.
158. The genetically engineered cell of any one of claims 150-157, wherein the cell further expresses one or more additional chimeric ligand receptors.
159. The genetically engineered cell of claim 158, wherein the chimeric ligand receptors are each distinct from one another.
160. The genetically engineered cell of claim 159, wherein the ligand binding domains of each chimeric ligand receptor bind a different ligand.
161. A genetically engineered cell expressing two or more chimeric ligand receptors of any one of claims 1-73.
162. The genetically engineered cell of claim 161, wherein the chimeric ligand receptors are each distinct from one another.
163. The genetically engineered cell of claim 162, wherein the ligand binding domains of each chimeric ligand receptor bind a different ligand.
164. The genetically engineered cell of any one of claims 158-163, wherein the cell further comprises two or more engineered transgenes, wherein each transgene comprises a synthetic promoter operably linked to a polynucleotide comprising a nucleic acid sequence encoding a target product.
165. The genetically engineered cell of claim 164, wherein each synthetic promoter is responsive to intracellular signaling from a distinct chimeric ligand receptor from the two or more chimeric ligand receptors expressed on the cell.
166. The genetically engineered cell of claim 164 or claim 165, wherein each target product is independently selected from the group consisting of a therapeutic molecule, a prophylactic molecule, and a diagnostic molecule.
167. The genetically engineered cell of any one of claims 150-166, wherein the cell is a mammalian cell.
168. The genetically engineered cell of claim 167, wherein the mammalian cell is a stem cell or a neuronal cell.
169. The genetically engineered cell of claim 168, wherein the stem cell is selected from the group consisting of an adult stem cell, an iPS cell, a bone marrow stem cell, a peripheral blood stem cell, and a mesenchymal stem cell (MSC).
170. The genetically engineered cell of claim 167, wherein the mammalian cell is an immune cell.
171. The genetically engineered cell of claim 170, wherein the immune cell is selected from the group consisting of a T cell, a B cell, an NK cell, a monocyte, a macrophage, an innate lymphoid cell, a mast cell, an eosinophil, a basophil, a neutrophil, and a dendritic cell.
172. The genetically engineered cell of claim 170, wherein the immune cell is a T cell.
173. The genetically engineered cell of claim 172, wherein the T cell is selected from the group consisting of a helper T cell, a cytotoxic T cell, a memory T cell, a regulatory T cell, a natural killer T cell, and a gamma delta T cell.
174. A method comprising contacting the chimeric ligand receptor of any one of claims 1-73 or the genetically engineered cell of any one of claims 150-173 with a biological tissue or biological fluid.
175. The method of claim 174, wherein the biological tissue or biological fluid is in a subject or is obtained from a subject.
176. The method of claim 175, wherein the subject has been diagnosed with, is at risk of developing, or is suspected of having a medical condition, optionally wherein the medical condition is a cancer or inflammatory condition.
177. A method of activating a signaling pathway, comprising:
   contacting the chimeric ligand receptor of any one of claims 1-73 or the genetically engineered cell of any one of claims 150-173 with a cognate ligand under conditions suitable for the chimeric ligand receptor to bind the cognate ligand,
   wherein binding of the cognate ligand with the chimeric ligand receptor induces a conformational reorganization of the multimerized scaffold domains that activates the intracellular signaling domains.
178. The method of claim 177, further comprising administering the cognate ligand to a surface of a cell.
179. A method of producing a genetically engineered cell expressing a chimeric ligand receptor, comprising:
   transfecting the isolated polynucleotide or set of isolated polynucleotides of any one of claims 74-148 or the vector or set of vectors of claim 149 into a cell; and
   inducing expression of the chimeric ligand receptor in the cell.
180. The method of claim 179, further comprising:
   transfecting into the cell an isolated polynucleotide comprising a synthetic promoter operably linked to a nucleic acid sequence encoding a target product.
181. The method of claim 180, wherein the synthetic promoter is responsive to intracellular signaling from the chimeric ligand receptor.
182. The method of any one of claims 179-181, wherein inducing expression of the chimeric ligand receptor comprises culturing the cell under conditions suitable for the cell to express the chimeric ligand receptor on a cell membrane of the cell.
183. A method comprising transfecting a cell with the polynucleotide or set of isolated polynucleotides of any one of claims 74-148 or the vector or set of vectors of claim 149, optionally wherein the cell is a mammalian cell, and optionally wherein the mammalian cell is an immune cell.
184. A kit comprising a polynucleotide comprising a nucleic acid sequence encoding at least one chimeric ligand receptor of any one of claims 1-73.
185. The kit of claim 184, further comprising at least one engineered transgene comprising a synthetic promoter operably linked to a polynucleotide comprising a nucleic acid sequence encoding a target product.
186. A chimeric ligand receptor comprising a receptor subunit, wherein the receptor subunit comprises a scaffold domain;
   wherein the scaffold domain comprises an extracellular domain and a transmembrane domain;
   wherein the extracellular domain is operably linked to a ligand binding domain;
   wherein the ligand binding domain binds to a ligand displayed on a surface or expressed on a cell surface;
   wherein the transmembrane domain is operably linked to an intracellular signaling domain;
   wherein the receptor subunit multimerizes via its scaffold domain in the presence of one or more additional receptor subunits; and
   wherein the multimerized receptor subunits undergo a conformational reorganization upon ligand binding to the chimeric ligand receptor.
187. The chimeric ligand receptor of claim 186, further comprising one or more additional receptor subunits, wherein each additional receptor subunit comprises a scaffold domain;
   wherein the scaffold domain of each additional receptor subunit comprises an extracellular domain and a transmembrane domain;
   wherein the extracellular domain of each additional receptor subunit is operably linked to a ligand binding domain;
   wherein the ligand binding domain binds to a ligand displayed on a surface or expressed on a cell surface; and
   wherein the transmembrane domain of each additional receptor subunit is operably linked to an intracellular signaling domain.
188. The chimeric ligand receptor of claim 187, wherein the receptor subunits multimerize via their scaffold domains to form the chimeric ligand receptor.
189. The chimeric ligand receptor of claim 188, wherein the multimerized receptor subunits undergo a conformational reorganization upon ligand binding to the chimeric receptor.
190. The chimeric ligand receptor of any one of claims 187-189, wherein the chimeric ligand binding domains of each receptor subunit bind the same ligand.
191. A chimeric ligand receptor comprising two or more receptor subunits,
   wherein each receptor subunit comprises a scaffold domain;
   wherein the receptor subunits multimerize via their scaffold domains to form the chimeric ligand receptor;
   wherein each scaffold domain comprises an extracellular domain and a transmembrane domain;
   wherein the extracellular domain is operably linked to a ligand binding domain, wherein the ligand binding domains of each receptor subunit bind the same ligand,
   wherein the ligand is displayed on a surface or expressed on a cell surface;
   wherein the transmembrane domain is operably linked to an intracellular signaling domain; and
   wherein binding of the ligand induces a conformational reorganization of the multimerized receptor subunits.
192. The chimeric ligand receptor of any one of claims 188-191, wherein multimerization of the receptor subunits occurs prior to ligand binding.
193. The chimeric ligand receptor of any one of claims 188-192, wherein the multimerized receptor subunits comprise a dimer, a trimer, tetramer, pentamer, or hexamer.
194. The chimeric ligand receptor of any one of claims 188-193 wherein the multimerized receptor subunits comprise a dimer.
195. The chimeric ligand receptor of any one of claims 189-194, wherein the conformational reorganization comprises a rotation of each scaffold domain around its own axis.
196. The chimeric ligand receptor of any one of claims 189-195, wherein the conformational reorganization activates the intracellular signaling domains of each receptor subunit.
197. The chimeric ligand receptor of any one of claims 189-195, wherein the conformational reorganization inhibits the intracellular signaling domains of each receptor subunit.
198. The chimeric ligand receptor of any one of claims 186-197, wherein the scaffold domain comprises the extracellular domain and transmembrane domain of a cytokine receptor.
199. The chimeric ligand receptor of any one of claims 186-198, wherein the scaffold domain comprises the extracellular domain and transmembrane domain of an erythropoietin receptor (EpoR).
200. The chimeric ligand receptor of claim 199, wherein the scaffold domain is inert to erythropoietin.
201. The chimeric ligand receptor of claim 199 or claim 200, wherein the scaffold domain comprises an extracellular domain and transmembrane domain comprising an amino acid sequence having 90% or greater sequence identity to SEQ ID NO: 8.
202. The chimeric ligand receptor of any one of claims 186-198, wherein the scaffold domain comprises the extracellular domain and transmembrane domain of a protein selected from the group consisting of a leptin receptor (Ob-R), a growth hormone receptor (GHR), an epidermal growth factor receptor (EGFR), Irk-B and p75^{NTR}.
203. The chimeric ligand receptor of claim 202, wherein the scaffold domain is inert to leptin, growth hormone, epidermal growth factor (EGF), or brain-derived neurotrophic factor (BDNF).
204. The chimeric ligand receptor of claim 202 or claim 203, wherein the scaffold domain comprises an extracellular domain and transmembrane domain comprising an amino acid sequence having 90% or greater sequence identity to a sequence selected from the group consisting of SEQ ID NO: 12, SEQ ID NO: 16, SEQ ID NO: 20, SEQ ID NO: 24, and SEQ ID NO: 28.
205. The chimeric ligand receptor of any one of claims 186-204, wherein the extracellular domain, the transmembrane domain, or both the extracellular domain and transmembrane domain of the scaffold domain comprise one or more modifications.
206. The chimeric ligand receptor of claim 186-205, wherein the extracellular domain comprises one or more amino acid substitutions, optionally wherein the extracellular domain comprises an F93A amino acid substitution.
207. The chimeric ligand receptor of any one of claims 186-206, wherein one or more additional amino acid residues are inserted adjacent to the transmembrane domain.
208. The chimeric ligand receptor of any one of claims 186-206, wherein one or more additional amino acid residues are inserted within the transmembrane domain.
209. The chimeric ligand receptor of claim 207 or claim 208, wherein the one or more additional amino acid residues are alanine residues.
210. The chimeric ligand receptor of claim 209, wherein the transmembrane domain further comprises one, two, three, or four additional alanine residues.
211. The chimeric ligand receptor of any one of claims 207-210, wherein the one or more additional amino acid residues are inserted C-terminal to the transmembrane domain.
212. The chimeric ligand receptor of any one of claims 186-211, wherein the ligand binding domain is linked to the extracellular domain through an extracellular linker region.
213. The chimeric ligand receptor of claim 212, wherein the extracellular linker region comprises one or more amino acid residues, optionally wherein the one or more amino acid residues comprise amino acids residues Serine-Glycine-Glutamic acid-Phenylalanine.
214. The chimeric ligand receptor of any one of claims 186-213, wherein the ligand binding domain does not bind a native ligand of the scaffold domain.
215. The chimeric ligand receptor of any one of claims 186-213, wherein the ligand binding domain does not bind erythropoietin.
216. The chimeric ligand receptor of any one of claims 186-213, wherein the ligand binding domain does not bind a ligand selected from the group consisting of leptin, growth hormone, epidermal growth factor (EGF), and brain-derived neurotrophic factor (BNDF).
217. The chimeric ligand receptor of any one of claims 186-213, wherein the ligand binding domain is not derived from a cytokine receptor.
218. The chimeric ligand receptor of any one of claims 186-217, wherein the ligand binding domain binds to a ligand selected from the group consisting of a protein complex, a protein, a peptide, a nucleic acid, a small molecule, and a chemical agent.
219. The chimeric ligand receptor of claim 218, wherein the ligand is selected from the group consisting of an antigen, a cytokine, a survival factor, a chemokine, a hormone, a transmitter, a growth factor, extracellular matrix, and a death factor.
220. The chimeric ligand receptor of any one of claims 186-219, wherein the ligand binding domain binds to an antigen.
221. The chimeric ligand receptor of claim 220, wherein the antigen is a tumor-associated antigen.
222. The chimeric ligand receptor of claim 221, wherein the tumor-associated antigen is selected from the group consisting of MS4A3, VSTM1, LAT2, CD131, GAPT, PRAM1, SLC22A16, SLC17A9, SPNS3, FLT3, CD33, CLEC12A, ADGRE2, IL3RA, CD117, CD93, IL1RAP, CD244, CCR1, LILRB2, PIEZO1, CD38, EMB, MYADM, LILRA2, CD300LF, CD70, 5T4, ADAM9, AFP, AXL, B7-H3, B7-H4, B7-H6, C4.4, CA6, Cadherin 3, Cadherin 6, CCR4, CD123, CD133, CD138, CD142, CD166, CD25, CD30, CD352, CD37, CD38, CD44, CD56, CD66e, CD71, CD74, CD79b, CD80, CEA, CEACAM5, Claudin18.2, cMet, CSPG4, CTLA, DLK1, DLL3, DR5, EGFR, ENPP3, EpCAM, EphA2, Ephrin A4, ETBR, FGFR2, FGFR3, FRalpha, FRb, GCC, GD2, GFRa4, gpA33, GPC3, gpNBM, GPRC5, HER2, IL-13R, IL-13Ra2, IL-8, IL-15, IL1RAP, Integrin aV, KIT, L1CAM, LAMP1, Lewis Y, LeY, LIV-1, LRRC, LY6E, MCSP, Mesothelin, MUC1, MUC16, MUC1C, NaPi2B, Nectin 4, NKG2D, NOTCH3, NY ESO 1, Ovarin, P-cadherin, pan-Erb2, PSCA, PSMA, PTK7, ROR1, S Aures, SCT, SLAMF7, SLITRK6, SSTR2, STEAP1, Survivin, TDGF1, TIM1, TROP2, and WT1.
223. The chimeric ligand receptor of any one of claims 186-222, wherein the ligand binding domain comprises an antibody, or antigen-binding fragment thereof.
224. The chimeric ligand receptor of claim 223, wherein the ligand binding domain comprises a single chain variable fragment (scFv), or a single-domain antibody (sdAb).
225. The chimeric ligand receptor of any one of claims 186-223, wherein each of the ligand binding domains comprises a single chain variable fragment (scFv), optionally wherein each scFv specifically binds to a distinct epitope of the antigen.
226. The chimeric ligand receptor of any one of claims 187-225, wherein the ligand binding domains of each receptor subunit are distinct from one another.
227. The chimeric ligand receptor of claim 226, wherein the chimeric ligand receptor comprises two ligand binding domains, and wherein one ligand binding domain comprises an immunoglobulin heavy chain variable domain (V_{H}) and the second ligand binding domain comprises an immunoglobulin light chain variable domain (V_{L}).
228. The chimeric ligand receptor of any one of claims 187-225, wherein the ligand binding domains of each receptor subunit are the same as one another.
229. The chimeric ligand receptor of claim 223, wherein the antibody, or antigen-binding fragment thereof, is a single-domain VHH camelid antibody.
230. The chimeric ligand receptor of any one of claims 186-229, wherein the intracellular signaling domain is inert to native ligand binding of the scaffold domain.
231. The chimeric ligand receptor of any one of claims 186-230, wherein the intracellular signaling domain does not comprise an endogenous intracellular signaling domain of the scaffold domain.
232. The chimeric ligand receptor of any one of claims 186-230, wherein the intracellular signaling domain does not comprise an endogenous erythropoietin receptor (EpoR) intracellular signaling domain.
233. The chimeric ligand receptor of any one of claims 186-230, wherein the intracellular signaling domain does not comprise an intracellular signaling domain selected from the group consisting of an endogenous leptin receptor (Ob-R) intracellular signaling domain, an endogenous growth hormone receptor (GHR) intracellular signaling domain, an endogenous epidermal growth factor receptor (EGFR) intracellular signaling domain, an endogenous Trk-B intracellular signaling domain, and an endogenous p75^{NTR} intracellular signaling domain.
234. The chimeric ligand receptor of any one of claims 186-230, wherein the intracellular signaling domain does not comprise an endogenous cytokine receptor intracellular signaling domain.
235. The chimeric ligand receptor of any one of claims 186-234, wherein the intracellular signaling domain induces downstream signaling via a JAK/STAT (Janus kinase/signal transducer and activator of transcription) signaling pathway, a MAPK (mitogen-activated protein kinase) signaling pathway, a PLCG (phospholipase C gamma) signaling pathway, or a PI3K/Akt (phosphatidylinositol 3-kinase/protein kinase B) signaling pathway.
236. The chimeric ligand receptor of any one of claims 186-234, wherein the intracellular signaling domain is selected from the group consisting of an intracellular signal transduction domain of IL-6RB (interleukin 6 receptor B), an intracellular signal transduction domain of FGFR1 (fibroblast growth factor receptor 1), and an intracellular signal transduction domain of VEGFR2 (vascular endothelial growth factor receptor 2).
237. The chimeric ligand receptor of any one of claims 186-234, wherein the intracellular signaling domain is an intracellular signal transduction domain of IL-6RB and induces downstream signaling via the JAK/STAT signaling pathway.
238. The chimeric ligand receptor of any one of claims 186-234, wherein the intracellular signaling domain is an intracellular signal transduction domain of FGFR1 and induces downstream signaling via the MAPK signaling pathway.
239. The chimeric ligand receptor of any one of claims 186-234, wherein the intracellular signaling domain is an intracellular signal transduction domain of VEGFR2 and induces downstream signaling via the PLCG signaling pathway.
240. The chimeric ligand receptor of any one of claims 186-234, wherein the intracellular signaling domain is an intracellular signal transduction domain of VEGER2 and induces downstream signaling via the PI3K/Akt signaling pathways.
241. chimeric ligand receptor of any one of claims 186-234, wherein the intracellular signaling domain is an intracellular signal transduction domain derived from an IL-2 signaling pathway, an IL-7 signaling pathway, or an IL-15 signaling pathway.
242. The chimeric ligand receptor of any one of claims 186-241, wherein the intracellular signaling domain comprises one or more modifications that modulate signaling activity of the intracellular signaling domain, optionally wherein the one or more modifications are one or more amino acid substitutions.
243. An isolated polynucleotide or a set of isolated polynucleotides encoding the chimeric ligand receptor of any one of claims 186-242.
244. The isolated polynucleotide or set of isolated polynucleotides of claim 243, comprising the cDNA of the chimeric ligand receptor of any one of claims 186-242.
245. An isolated polynucleotide or a set of isolated polynucleotides comprising a nucleic acid sequence encoding a chimeric ligand receptor subunit, wherein the receptor subunit comprises a scaffold domain capable of multimerizing the receptor subunit in the presence of one or more additional receptor subunits and wherein the receptor subunit is capable of undergoing a conformational reorganization induced by ligand binding when multimerized;
   wherein the scaffold domain comprises an extracellular domain and a transmembrane domain;
   wherein the extracellular domain is operably linked to a ligand binding domain; wherein the ligand binding domain binds to a ligand displayed on a surface or expressed on a cell surface; and
   wherein the transmembrane domain is operably linked to an intracellular signaling domain.
246. The isolated polynucleotide or set of isolated polynucleotides of claim 245, further comprising one or more nucleic acid sequences, wherein each additional nucleic acid sequence encoding an additional chimeric ligand receptor subunit;
   wherein each additional receptor subunit comprises a scaffold domain;
   wherein the scaffold domain of each additional receptor subunit comprises an extracellular domain and a transmembrane domain;
   wherein the extracellular domain of each additional receptor subunit is operably linked to a ligand binding domain;
   wherein the ligand binding domain binds to a ligand displayed on a surface or expressed on a cell surface; and
   wherein the transmembrane domain of each additional receptor subunit is operably linked to an intracellular signaling domain.
247. The isolated polynucleotide or set of isolated polynucleotides of claim 245 or claim 246, wherein the receptor subunits multimerize via their scaffold domains to form the chimeric ligand receptor.
248. The isolated polynucleotide or set of isolated polynucleotides of any one of claims 245-247, wherein the multimerized receptor subunits undergo a conformational reorganization upon ligand binding to the chimeric receptor.
249. The isolated polynucleotide or set of isolated polynucleotides of any one of claims 245-248 wherein the chimeric ligand binding domains of each receptor subunit bind the same ligand.
250. An isolated polynucleotide or a set of isolated polynucleotides comprising two or more nucleic acid sequences, wherein each nucleic acid sequence encoding a chimeric ligand receptor subunit;
   wherein each receptor subunit comprises a scaffold domain; wherein the receptor subunits multimerize via their scaffold domains to form the chimeric ligand receptor;
   wherein each scaffold domain comprises an extracellular domain and a transmembrane domain;
   wherein the extracellular domain is operably linked to a ligand binding domain, wherein the ligand binding domains of each receptor subunit bind the same ligand;
   wherein the ligand is displayed on a surface or expressed on a cell surface;
   wherein the transmembrane domain is operably linked to an intracellular signaling domain; and
   wherein binding of the ligand induces a conformational reorganization of the multimerized receptor subunits.
251. The isolated polynucleotide or set of isolated polynucleotides of any one of claims 247-250, wherein multimerization of the receptor subunits occurs prior to ligand binding.
252. The isolated polynucleotide or set of isolated polynucleotides of any one of claims 247-251, wherein the multimerized receptor subunits comprise a dimer, a trimer, tetramer, pentamer, or hexamer.
253. The isolated polynucleotide or set of isolated polynucleotides of any one of claims 247-252, wherein the multimerized receptor subunits comprise a dimer.
254. The isolated polynucleotide or set of isolated polynucleotides of any one of claims 248-253, wherein the conformational reorganization comprises a rotation of each scaffold domain around its own axis.
255. The isolated polynucleotide or set of isolated polynucleotides of any one of claims 248-254 wherein the conformational reorganization activates the intracellular signaling domains of each receptor subunit.
256. The isolated polynucleotide or set of isolated polynucleotides of any one of claims 248-254, wherein the conformational reorganization inhibits the intracellular signaling domains of each receptor subunit.
257. The isolated polynucleotide or set of isolated polynucleotides of any one of claims 245-256, wherein the scaffold domain comprises the extracellular domain and transmembrane domain of a cytokine receptor.
258. The isolated polynucleotide or set of isolated polynucleotides of any one of claims 245-257, wherein the scaffold domain is inert to its native ligand.
259. The isolated polynucleotide or set of isolated polynucleotides of any one of claims 245-257, wherein the scaffold domain comprises the extracellular domain and transmembrane domain of an erythropoietin receptor (EpoR).
260. The isolated polynucleotide or set of isolated polynucleotides of claim 259, wherein the scaffold domain is inert to erythropoietin.
261. The isolated polynucleotide or set of isolated polynucleotides of claim 259 or claim 260, wherein the scaffold domain comprises an extracellular domain and transmembrane domain comprising an amino acid sequence having 90% or greater sequence identity to SEQ ID NO: 8.
262. The isolated polynucleotide or set of isolated polynucleotides of any one of claims 245-257, wherein the scaffold domain comprises the extracellular domain and transmembrane domain of a protein selected from the group consisting of a leptin receptor (Ob-R), a growth hormone receptor (GHR), an epidermal growth factor receptor (EGFR), Trk-B and p75^{NTR}.
263. The isolated polynucleotide or set of isolated polynucleotides of claim 262, wherein the scaffold domain is inert to leptin, growth hormone, epidermal growth factor (EGF), or brain-derived neurotrophic factor (BDNF).
264. The isolated polynucleotide or set of isolated polynucleotides of claim 262 or claim 263, wherein the scaffold domain comprises an extracellular domain and transmembrane domain comprising an amino acid sequence having 90% or greater sequence identity to a sequence selected from the group consisting of SEQ ID NO: 12, SEQ ID NO: 16, SEQ ID NO: 20, SEQ ID NO: 24, and SEQ ID NO: 28.
265. The isolated polynucleotide or set of isolated polynucleotides of any one of claims 245-264, wherein the extracellular domain, the transmembrane domain, or both the extracellular domain and transmembrane domain of the scaffold domain comprise one or more modifications.
266. The isolated polynucleotide or set of isolated polynucleotides of any one of claims 245-265, wherein the extracellular domain comprises one or more amino acid substitutions, optionally wherein the extracellular domain comprises an F93A amino acid substitution.
267. The isolated polynucleotide or set of isolated polynucleotides of any one of claims 245-266, wherein one or more additional amino acid residues are inserted adjacent to the transmembrane domain.
268. The isolated polynucleotide or set of isolated polynucleotides of any one of claims 245-266, wherein one or more additional amino acid residues are inserted within the transmembrane domain.
269. The isolated polynucleotide or set of isolated polynucleotides of claim 267 or claim 268, wherein the one or more additional amino acid residues are alanine residues.
270. The isolated polynucleotide or set of isolated polynucleotides of claim 269, wherein the transmembrane domain further comprises one, two, three, or four additional alanine residues.
271. The isolated polynucleotide or set of isolated polynucleotides of any one of claims 267-270, wherein the one or more additional amino acid residues are inserted C-terminal to the transmembrane domain.
272. The isolated polynucleotide or set of isolated polynucleotides of any one of claims 245-271, wherein the ligand binding domain is linked to the extracellular domain through an extracellular linker region.
273. The isolated polynucleotide or set of isolated polynucleotides of claim 272, wherein the extracellular linker region comprises one or more amino acid residues, optionally wherein the one or more amino acid residues comprise amino acids residues Serine-Glycine-Glutamic acid-Phenylalanine.
274. The isolated polynucleotide or set of isolated polynucleotides of any one of claims 245-273, wherein the ligand binding domain does not bind a native ligand of the scaffold domain.
275. The isolated polynucleotide or set of isolated polynucleotides of any one of claims 245-273, wherein the ligand binding domain does not bind erythropoietin.
276. The isolated polynucleotide or set of isolated polynucleotides of any one of claims 245-273, wherein the ligand binding domain does not bind a ligand selected from the group consisting of leptin, growth hormone, epidermal growth factor (EGF), and brain-derived neurotrophic factor (BDNF).
277. The isolated polynucleotide or set of isolated polynucleotides of any one of claims 245-273, wherein the ligand binding domain is not derived from a cytokine receptor.
278. The isolated polynucleotide or set of isolated polynucleotides of any one of claims 245-277, wherein the ligand binding domain binds to a ligand selected from the group consisting of a protein complex, a protein, a peptide, a nucleic acid, a small molecule, and a chemical agent.
279. The isolated polynucleotide or set of isolated polynucleotides of claim 278, wherein the ligand is selected from the group consisting of an antigen, a cytokine, a survival factor, a chemokine, a hormone, a transmitter, a growth factor, extracellular matrix, and a death factor.
280. The isolated polynucleotide or set of isolated polynucleotides of any one of claims 245-279, wherein the ligand binding domain binds to an antigen.
281. The isolated polynucleotide or set of isolated polynucleotides of claim 80, wherein the antigen is a tumor-associated antigen.
282. The isolated polynucleotide or set of isolated polynucleotides of claim 281, wherein the tumor-associated antigen is selected from the group consisting of MS4A3, VSTM1, LAT2, LCI, CD131, GAPT, PRAM1, SLC22A16, SLC17A9, SPNS3, FLT3, CD33, CLEC12A, ADGRE2, IL3RA, CD117, CD3, IL1RAP, CD244, CCR1, LILRB2, PIEZO1, CD38, EMB, MYADM, LILRA2, CD300LF, CD70, 5T4, ADAM9, AFP, AXL, B7-H3, B7-H4, B7-H6, C4.4, CA6, Cadherin 3, Cadherin 6, CCR4, CD123, CD133, CD138, CD142, CD166, CD25, CD30, CD352, CD37, CD38, CD44, CD56, CD66e, CD71, CD74, CD79b, CD80, CEA, CEACAM5, Claudin18.2, cMet, CSPG4, CTLA, DLK1, DLL3, DR5, EGFR, ENPP3, EpCAM, EphA2, Ephrin A4, ETBR, FGFR2, FGFR3, FRalpha, FRb, GCC, GD2, GFRa4, gpA33, GPC3, gpNBM, GPRC5, HER2, IL-13R, IL-13Ra2, IL-8, IL-15, IL1RAP, Integrin aV, KIT, L1CAM, LAMP1, Lewis Y, LeY, LIV-1, LRRC, LY6E, MCSP, Mesothelin, MUC1, MUC16, MUC1C, NaPi2B, Nectin 4, NKG2D, NOTCH3, NY ESO 1, Ovarin, P-cadherin, pan-Erb2, PSCA, PSMA, PTK7, ROR1, S Aures, SCT, SLAMF7, SLITRK6, SSTR2, STEAP1, Survivin, TDGF1, TIM1, TROP2, and WT1.
283. The isolated polynucleotide or set of isolated polynucleotides of of any one of claims 245-282, wherein the ligand binding domain comprises an antibody, or antigen-binding fragment thereof.
284. The isolated polynucleotide or set of isolated polynucleotides of claim 283, wherein the ligand binding domain comprises a single chain variable fragment (scFv), or a single-domain antibody (sdAb).
285. The isolated polynucleotide or set of isolated polynucleotides of any one of claims 245-283, wherein each of the ligand binding domains comprises a single chain variable fragment (scFv), optionally wherein each scFv specifically binds to a distinct epitope of the antigen.
286. The isolated polynucleotide or set of isolated polynucleotides of any one of claims 246-285, wherein the ligand binding domains of each receptor subunit are distinct from one another.
287. The isolated polynucleotide or set of isolated polynucleotides of claim 286, wherein the chimeric ligand receptor comprises two ligand binding domains, and wherein one ligand binding domain comprises an immunoglobulin heavy chain variable domain (V_{H}) and the second ligand binding domain comprises an immunoglobulin light chain variable domain (V_{L}).
288. The isolated polynucleotide or set of isolated polynucleotides of any one of claims 246-285, wherein the ligand binding domains of each receptor subunit are the same as one another.
289. The isolated polynucleotide or set of isolated polynucleotides of claim 283, wherein the antibody, or antigen-binding fragment thereof, is a single-domain VHH camelid antibody.
290. The isolated polynucleotide or set of isolated polynucleotides of any one of claims 245-289, wherein the intracellular signaling domain is inert to native ligand binding of the scaffold domain.
291. The isolated polynucleotide or set of isolated polynucleotides of any one of claims 245-290, wherein the intracellular signaling domain does not comprise an endogenous intracellular signaling domain of the scaffold domain.
292. The isolated polynucleotide or set of isolated polynucleotides of any one of claims 245-290, wherein the intracellular signaling domain does not comprise an endogenous erythropoietin receptor (EpoR) intracellular signaling domain.
293. The isolated polynucleotide or set of isolated polynucleotides of any one of claims 245-290, wherein the intracellular signaling domain does not comprise an intracellular signaling domain selected from the group consisting of an endogenous leptin receptor (Ob-R) intracellular signaling domain, an endogenous growth hormone receptor (GHR) intracellular signaling domain, an endogenous epidermal growth factor receptor (EGFR) intracellular signaling domain, an endogenous Trk-B intracellular signaling domain, and an endogenous p75^{NTR} intracellular signaling domain.
294. The isolated polynucleotide or set of isolated polynucleotides of any one of claims 245-290, wherein the intracellular signaling domain does not comprise an endogenous cytokine receptor intracellular signaling domain.
295. The isolated polynucleotide or set of isolated polynucleotides of any one of claims 245-294, wherein the intracellular signaling domain induces downstream signaling via a JAK/STAT (Janus kinase/signal transducer and activator of transcription) signaling pathway, a MAPK (mitogen-activated protein kinase) signaling pathway, a PLCG (phospholipase C gamma) signaling pathway, or a PI3K/Akt (phosphatidylinositol 3-kinase/protein kinase B) signaling pathway.
296. The isolated polynucleotide or set of isolated polynucleotides of any one of claims 245-294, wherein the intracellular signaling domain is selected from the group consisting of an intracellular signal transduction domain of IL-6RB (interleukin 6 receptor B), an intracellular signal transduction domain of FGFR1 (fibroblast growth factor receptor 1), and an intracellular signal transduction domain of VEGFR2 (vascular endothelial growth factor receptor 2).
297. The isolated polynucleotide or set of isolated polynucleotides of any one of claims 245-294 wherein the intracellular signaling domain is an intracellular signal transduction domain of IL-6RB and induces downstream signaling via the JAK/STAT signaling pathway.
298. The isolated polynucleotide or set of isolated polynucleotides of any one of claims 245-294, wherein the intracellular signaling domain is an intracellular signal transduction domain of FGFR1 and induces downstream signaling via the MAPK signaling pathway.
299. The isolated polynucleotide or set of isolated polynucleotides of any one of claims 245-294, wherein the intracellular signaling domain is an intracellular signal transduction domain of VEGFR2 and induces downstream signaling via the PLCG signaling pathway.
300. The isolated polynucleotide or set of isolated polynucleotides of any one of claims 245-294, wherein the intracellular signaling domain is an intracellular signal transduction domain of VEGFR2 and induces downstream signaling via the PI3K/Akt signaling pathways.
301. The isolated polynucleotide or set of isolated polynucleotides of any one of claims 245-294, wherein the intracellular signaling domain is an intracellular signal transduction domain derived from an IL-2 signaling pathway, an IL-7 signaling pathway, or an IL-15 signaling pathway.
302. The isolated polynucleotide or set of isolated polynucleotides of any one of claims 245-301, wherein the intracellular signaling domain comprises one or more modifications that modulate signaling activity of the intracellular signaling domain, optionally wherein the one or more modifications are one or more amino acid substitutions.
303. A vector or a set of vectors comprising the polynucleotide or set of polynucleotides of any one of claims 243-302.
304. A genetically engineered cell comprising the polynucleotide or set of polynucleotides of any one of claims 243-302 or the vector or set of vectors of claim 303.
305. A genetically engineered cell expressing the chimeric ligand receptor of any one of claims 186-242.
306. The genetically engineered cell of claim 304 or claim 305, wherein the cell further comprises an engineered transgene, wherein the transgene comprises a synthetic promoter operably linked to a polynucleotide comprising a nucleic acid sequence encoding a target product.
307. The genetically engineered cell of claim 306, wherein the synthetic promoter is responsive to intracellular signaling from the chimeric ligand receptor.
308. The genetically engineered cell of claim 306 or claim 307, wherein the target product is selected from the group consisting of a therapeutic molecule, a prophylactic molecule, and a diagnostic molecule.
309. The genetically engineered cell of any one of claims 306-308, wherein the target product is a chimeric antigen receptor (CAR).
310. The genetically engineered cell of any one of claims 306-308, wherein the target product is a proinflammatory cytokine.
311. The genetically engineered cell of claim 310, wherein the proinflammatory cytokine is selected from the group consisting of IL-2, IL-7, IL-12, IL-15, IL-18, and IL-21.
312. The genetically engineered cell of any one of claims 304-311, wherein the cell further expresses one or more additional chimeric ligand receptors.
313. The genetically engineered cell of claim 312, wherein the chimeric ligand receptors are each distinct from one another.
314. The genetically engineered cell of claim 313, wherein the ligand binding domains of each chimeric ligand receptor bind a different ligand.
315. A genetically engineered cell expressing two or more chimeric ligand receptors of any one of claims 186-242.
316. The genetically engineered cell of claim 315, wherein the chimeric ligand receptors are each distinct from one another.
317. The genetically engineered cell of claim 316, wherein the ligand binding domains of each chimeric ligand receptor bind a different ligand.
318. The genetically engineered cell of any one of claims 312-317, wherein the cell further comprises two or more engineered transgenes, wherein each transgene comprises a synthetic promoter operably linked to a polynucleotide comprising a nucleic acid sequence encoding a target product.
319. The genetically engineered cell of claim 318, wherein each synthetic promoter is responsive to intracellular signaling from a distinct chimeric ligand receptor from the two or more chimeric ligand receptors expressed on the cell.
320. The genetically engineered cell of claim 318 or claim 319, wherein each target product is independently selected from the group consisting of a therapeutic molecule, a prophylactic molecule, and a diagnostic molecule.
321. The genetically engineered cell of any one of claims 304-320, wherein the cell is a mammalian cell.
322. The genetically engineered cell of claim 321, wherein the mammalian cell is a stem cell or a neuronal cell.
323. The genetically engineered cell of claim 322, wherein the stem cell is selected from the group consisting of an adult stem cell, an iPS cell, a bone marrow stem cell, a peripheral blood stem cell, and a mesenchymal stem cell (MSC).
324. The genetically engineered cell of claim 321, wherein the mammalian cell is an immune cell.
325. The genetically engineered cell of claim 324, wherein the immune cell is selected from the group consisting of a T cell, a B cell, an NK cell, a monocyte, a macrophage, an innate lymphoid cell, a mast cell, an eosinophil, a basophil, a neutrophil, and a dendritic cell.
326. The genetically engineered cell of claim 324, wherein the immune cell is a T cell.
327. The genetically engineered cell of claim 326, wherein the T cell is selected from the group consisting of a helper T cell, a cytotoxic T cell, a memory T cell, a regulatory T cell, a natural killer T cell, and a gamma delta T cell.
328. A method comprising contacting the chimeric ligand receptor of any one of claims 186-242 or the genetically engineered cell of any one of claims 304-327 with a biological tissue or biological fluid.
329. The method of claim 328, wherein the biological tissue or biological fluid is in a subject or is obtained from a subject.
330. The method of claim 329, wherein the subject has been diagnosed with, is at risk of developing, or is suspected of having a medical condition, optionally wherein the medical condition is a cancer or inflammatory condition.
331. A method of activating a signaling pathway, comprising:
   contacting the chimeric ligand receptor of any one of claims 186-242 or the genetically engineered cell of any one of claims 304-327 with a cognate ligand under conditions suitable for the chimeric ligand receptor to bind the cognate ligand,
   wherein binding of the cognate ligand with the chimeric ligand receptor induces a conformational reorganization of the multimerized scaffold domains that activates the intracellular signaling domains.
332. The method of claim 331, further comprising administering the cognate ligand to a surface of a cell.
333. A method of producing a genetically engineered cell expressing a chimeric ligand receptor, comprising:
   transfecting the isolated polynucleotide or set of isolated polynucleotides of any one of claims 243-302 or the vector or set of vectors of claim 303 into a cell; and
   inducing expression of the chimeric ligand receptor in the cell.
334. The method of claim 333, further comprising:
   transfecting into the cell an isolated polynucleotide comprising a synthetic promoter operably linked to a nucleic acid sequence encoding a target product.
335. The method of claim 334, wherein the synthetic promoter is responsive to intracellular signaling from the chimeric ligand receptor.
336. The method of any one of claims 328-335, wherein inducing expression of the chimeric ligand receptor comprises culturing the cell under conditions suitable for the cell to express the chimeric ligand receptor on a cell membrane of the cell.
337. A method comprising transfecting a cell with the polynucleotide or set of isolated polynucleotides of any one of claims 243-302 or the vector or set of vectors of claim 303, optionally wherein the cell is a mammalian cell, and optionally wherein the mammalian cell is an immune cell.
338. A kit comprising a polynucleotide comprising a nucleic acid sequence encoding at least one chimeric ligand receptor of any one of claims 186-242.
339. The kit of claim 338, further comprising at least one engineered transgene comprising a synthetic promoter operably linked to a polynucleotide comprising a nucleic acid sequence encoding a target product.
340. A chimeric ligand receptor comprising a receptor subunit, wherein the receptor subunit comprises a scaffold domain;
   wherein the scaffold domain comprises an extracellular domain and a transmembrane domain;
   wherein the extracellular domain is operably linked to a ligand binding domain;
   wherein the transmembrane domain is operably linked to an intracellular signaling domain;
   wherein the receptor subunit multimerizes via its scaffold domain in the presence of one or more additional receptor subunits;
   wherein the multimerized receptor subunits undergo a conformational reorganization upon ligand binding to the chimeric ligand receptor; and
   wherein the conformational reorganization inhibits the intracellular signaling domains of each receptor subunit.
341. The chimeric ligand receptor of claim 340, further comprising one or more additional receptor subunits, wherein each additional receptor subunit comprises a scaffold domain;
   wherein the scaffold domain of each additional receptor subunit comprises an extracellular domain and a transmembrane domain;
   wherein the extracellular domain of each additional receptor subunit is operably linked to a ligand binding domain;
   wherein the transmembrane domain of each additional receptor subunit is operably linked to an intracellular signaling domain.
342. The chimeric ligand receptor of claim 341, wherein the receptor subunits multimerize via their scaffold domains to form the chimeric ligand receptor.
343. The chimeric ligand receptor of claim 342, wherein the multimerized receptor subunits undergo a conformational reorganization upon ligand binding to the chimeric receptor and wherein the conformational reorganization inhibits the intracellular signaling domains of each receptor subunit.
344. The chimeric ligand receptor of any one of claims 340-343, wherein the chimeric ligand binding domains of each receptor subunit bind the same ligand.
345. A chimeric ligand receptor comprising two or more receptor subunits,
   wherein each receptor subunit comprises a scaffold domain;
   wherein the receptor subunits multimerize via their scaffold domains to form the chimeric ligand receptor;
   wherein each scaffold domain comprises an extracellular domain and a transmembrane domain;
   wherein the extracellular domain is operably linked to a ligand binding domain, wherein the ligand binding domains of each receptor subunit bind the same ligand,
   wherein the transmembrane domain is operably linked to an intracellular signaling domain;
   wherein binding of the ligand induces a conformational reorganization of the multimerized receptor subunits; and
   wherein the conformational reorganization inhibits the intracellular signaling domains of each receptor subunit.
346. The chimeric ligand receptor of any one of claims 342-345, wherein multimerization of the receptor subunits occurs prior to ligand binding.
347. The chimeric ligand receptor of any one of claims 342-346, wherein the multimerized receptor subunits comprise a dimer, a trimer, tetramer, pentamer, or hexamer.
348. The chimeric ligand receptor of any one of claims 342-347, wherein the multimerized receptor subunits comprise a dimer.
349. The chimeric ligand receptor of any one of claims 343-348, wherein the conformational reorganization comprises a rotation of each scaffold domain around its own axis.
350. The chimeric ligand receptor of any one of claims 340-349 wherein the scaffold domain comprises the extracellular domain and transmembrane domain of a cytokine receptor.
351. The chimeric ligand receptor of any one of claims 340-350, wherein the scaffold domain comprises the extracellular domain and transmembrane domain of an erythropoietin receptor (EpoR).
352. The chimeric ligand receptor of claim 351, wherein the scaffold domain is inert to erythropoietin.
353. The chimeric ligand receptor of claim 351 or claim 352, wherein the scaffold domain comprises an extracellular domain and transmembrane domain comprising an amino acid sequence having 90% or greater sequence identity to SEQ ID NO: 8.
354. The chimeric ligand receptor of any one of claims 340-350, wherein the scaffold domain comprises the extracellular domain and transmembrane domain of a protein selected from the group consisting of a leptin receptor (Ob-R), a growth hormone receptor (GHR), an epidermal growth factor receptor (EGFR), Irk-B and p75^{NTR}.
355. The chimeric ligand receptor of claim 354, wherein the scaffold domain is inert to leptin, growth hormone, epidermal growth factor (EGF), or brain-derived neurotrophic factor (BDNF).
356. The chimeric ligand receptor of claim 354 or claim 355 wherein the scaffold domain comprises an extracellular domain and transmembrane domain comprising an amino acid sequence having 90% or greater sequence identity to a sequence selected from the group consisting of SEQ ID NO: 12, SEQ ID NO: 16, SEQ ID NO: 20, SEQ ID NO: 24, and SEQ ID NO: 28.
357. The chimeric ligand receptor of any one of claims 340-356, wherein the extracellular domain, the transmembrane domain, or both the extracellular domain and transmembrane domain of the scaffold domain comprise one or more modifications.
358. The chimeric ligand receptor of claim 340-357, wherein the extracellular domain comprises one or more amino acid substitutions, optionally wherein the extracellular domain comprises an F93A amino acid substitution.
359. The chimeric ligand receptor of any one of claims 340-358, wherein one or more additional amino acid residues are inserted adjacent to the transmembrane domain.
360. The chimeric ligand receptor of any one of claims 340-358, wherein one or more additional amino acid residues are inserted within the transmembrane domain.
361. The chimeric ligand receptor of claim 359 or claim 360, wherein the one or more additional amino acid residues are alanine residues.
362. The chimeric ligand receptor of claim 361, wherein the transmembrane domain further comprises one, two, three, or four additional alanine residues.
363. The chimeric ligand receptor of any one of claims 359-362, wherein the one or more additional amino acid residues are inserted C-terminal to the transmembrane domain.
364. The chimeric ligand receptor of any one of claims 340-363, wherein the ligand binding domain is linked to the extracellular domain through an extracellular linker region.
365. The chimeric ligand receptor of claim 364, wherein the extracellular linker region comprises one or more amino acid residues, optionally wherein the one or more amino acid residues comprise amino acids residues Serine-Glycine-Glutamic acid-Phenylalanine.
366. The chimeric ligand receptor of any one of claims 340-365 wherein the ligand binding domain does not bind a native ligand of the scaffold domain.
367. The chimeric ligand receptor of any one of claims 340-365, wherein the ligand binding domain does not bind erythropoietin.
368. The chimeric ligand receptor of any one of claims 340-365, wherein the ligand binding domain does not bind a ligand selected from the group consisting of leptin, growth hormone, epidermal growth factor (EGF), and brain-derived neurotrophic factor (BDNF).
369. The chimeric ligand receptor of any one of claims 340-365, wherein the ligand binding domain is not derived from a cytokine receptor.
370. The chimeric ligand receptor of any one of claims 340-369, wherein the ligand binding domain binds to a ligand selected from the group consisting of a protein complex, a protein, a peptide, a nucleic acid, a small molecule, and a chemical agent.
371. The chimeric ligand receptor of claim 370, wherein the ligand is selected from the group consisting of an antigen, a cytokine, a survival factor, a chemokine, a hormone, a transmitter, a growth factor, extracellular matrix, and a death factor.
372. The chimeric ligand receptor of any one of claims 340-371, wherein the ligand binding domain binds to a ligand displayed on a surface or expressed on a cell surface.
373. The chimeric ligand receptor of claim 372, wherein the the ligand expressed on a cell surface is a tumor-associated antigen.
374. The chimeric ligand receptor of claim 373, wherein the tumor-associated antigen is selected from the group consisting of MS4A3, VSTM1, LAT2, MLC1, CD131, GAPT, PRAM1, SLC22A16, SLC17A9, SPNS3, FLT3, CD33, CLEC12A, ADGRE2, IL3RA, CD117, CD93, IL1RAP, CD244, CCR1, LILRB2, PIEZO1, CD38, EMB, MYADM, LILRA2, CD300LF, CD70, 5T4, ADAM9, AFP, AXL, B7-H3, B7-H4, B7-H6, C4.4, CA6, Cadherin 3, Cadherin 6, CCR4, CD123, CD133, CD138, CD142, CD166, CD25, CD30, CD352, CD37, CD38, CD44, CD56, CD66e, CD71, CD74, CD79b, CD80, CEA, CEACAM5, Claudin18.2, cMet, CSPG4, CTLA, DLK1, DLL3, DR5, EGFR, ENPP3, EpCAM, EphA2, Ephrin A4, ETBR, FGFR2, FGFR3, FRalpha, FRb, GCC, GD2, GFRa4, gpA33, GPC3, gpNBM, GPRC5, HER2, IL-13R, IL-13Ra2, IL-8, IL-15, IL1RAP, Integrin aV, KIT, L1CAM, LAMP1, Lewis Y, LeY, LIV-1, LRRC, LY6E, MCSP, Mesothelin, MUC1, MUC16, MUC1C, NaPi2B, Nectin 4, NKG2D, NOTCH3, NY ESO 1, Ovarin, P-cadherin, pan-Erb2, PSCA, PSMA, PTK7, ROR1, S Aures, SCT, SLAMF7, SLITRK6, SSTR2, STEAP1, Survivin, TDGF1, TIM1, TROP2, and WT1.
375. The chimeric ligand receptor of any one of claims 340-371, wherein the ligand binding domain binds to a soluble ligand.
376. The chimeric ligand receptor of claim 375, wherein the soluble ligand is a secreted cytokine.
377. The chimeric ligand receptor of claim 376, wherein the secreted cytokine is an immunosuppressive cytokine.
378. The chimeric ligand receptor of claim 376, wherein the secreted cytokine is selected from the group consisting of TGFbeta, IL-10, IL-6, IL-23, IL-8, CCL2, CXCL12, CXCL8, CXCL1, CXCL2, CXCL3, CXCL8, CXCL13, CCL5, CCL17, and CCL22.
379. The chimeric ligand receptor of any one of claims 340-378, wherein the ligand binding domain binds to an antigen.
380. The chimeric ligand receptor of claim 379, wherein the ligand binding domain comprises an antibody, or antigen-binding fragment thereof.
381. The chimeric ligand receptor of claim 380, wherein the ligand binding domain comprises a single chain variable fragment (scFv), or a single-domain antibody (sdAb).
382. The chimeric ligand receptor of any one of claims 340-380, wherein each of the ligand binding domains comprises a single chain variable fragment (scFv), optionally wherein each scFv specifically binds to a distinct epitope of the antigen.
383. The chimeric ligand receptor of any one of claims 340-382, wherein the ligand binding domains of each receptor subunit are distinct from one another.
384. The chimeric ligand receptor of claim 383, wherein the chimeric ligand receptor comprises two ligand binding domains, and wherein one ligand binding domain comprises an immunoglobulin heavy chain variable domain (V_{H}) and the second ligand binding domain comprises an immunoglobulin light chain variable domain (V_{L}).
385. The chimeric ligand receptor of any one of claims 341-382, wherein the ligand binding domains of each receptor subunit are the same as one another.
386. The chimeric ligand receptor of claim 380, wherein the antibody, or antigen-binding fragment thereof, is a single-domain VHH camelid antibody.
387. The chimeric ligand receptor of any one of claims 340-386, wherein the intracellular signaling domain is inert to native ligand binding of the scaffold domain.
388. The chimeric ligand receptor of any one of claims 340-387, wherein the intracellular signaling domain does not comprise an endogenous intracellular signaling domain of the scaffold domain.
389. The chimeric ligand receptor of any one of claims 340-387 wherein the intracellular signaling domain does not comprise an endogenous erythropoietin receptor (EpoR) intracellular signaling domain.
390. The chimeric ligand receptor of any one of claims 340-387, wherein the intracellular signaling domain does not comprise an intracellular signaling domain selected from the group consisting of an endogenous leptin receptor (Ob-R) intracellular signaling domain, an endogenous growth hormone receptor (GHR) intracellular signaling domain, an endogenous epidermal growth factor receptor (EGFR) intracellular signaling domain, an endogenous Trk-B intracellular signaling domain, and an endogenous p75^{NTR} intracellular signaling domain.
391. The chimeric ligand receptor of any one of claims 340-387, wherein the intracellular signaling domain does not comprise an endogenous cytokine receptor intracellular signaling domain.
392. The chimeric ligand receptor of any one of claims 340-391, wherein the intracellular signaling domain induces downstream signaling via a JAK/STAT (Janus kinase/signal transducer and activator of transcription) signaling pathway, a MAPK (mitogen-activated protein kinase) signaling pathway, a PLCG (phospholipase C gamma) signaling pathway, or a PI3K/Akt (phosphatidylinositol 3-kinase/protein kinase B) signaling pathway.
393. The chimeric ligand receptor of any one of claims 340-391, wherein the intracellular signaling domain is selected from the group consisting of an intracellular signal transduction domain of IL-6RB (interleukin 6 receptor B), an intracellular signal transduction domain of FGFR1 (fibroblast growth factor receptor 1), and an intracellular signal transduction domain of VEGFR2 (vascular endothelial growth factor receptor 2).
394. The chimeric ligand receptor of any one of claims 340-391, wherein the intracellular signaling domain is an intracellular signal transduction domain of IL-6RB and induces downstream signaling via the JAK/STAT signaling pathway.
395. The chimeric ligand receptor of any one of claims 340-391, wherein the intracellular signaling domain is an intracellular signal transduction domain of FGFR1 and induces downstream signaling via the MAPK signaling pathway.
396. The chimeric ligand receptor of any one of claims 340-391, wherein the intracellular signaling domain is an intracellular signal transduction domain of VEGFR2 and induces downstream signaling via the PLCG signaling pathway.
397. The chimeric ligand receptor of any one of claims 340-391, wherein the intracellular signaling domain is an intracellular signal transduction domain of VEGFR2 and induces downstream signaling via the PI3K/Akt signaling pathways.
398. The chimeric ligand receptor of any one of claims 340-391, wherein the intracellular signaling domain is an intracellular signal transduction domain derived from an IL-2 signaling pathway, an IL-7 signaling pathway, or an IL-15 signaling pathway.
399. The chimeric ligand receptor of any one of claims 340-398, wherein the intracellular signaling domain comprises one or more modifications that modulate signaling activity of the intracellular signaling domain, optionally wherein the one or more modifications are one or more amino acid substitutions.
400. An isolated polynucleotide or a set of isolated polynucleotides encoding the chimeric ligand receptor of any one of claims 340-399.
401. The isolated polynucleotide or set of isolated polynucleotides of claim 400, comprising the cDNA of the chimeric ligand receptor of any one of claims 340-399
402. An isolated polynucleotide or a set of isolated polynucleotides comprising a nucleic acid sequence encoding a chimeric ligand receptor subunit, wherein the receptor subunit comprises a scaffold domain capable of multimerizing the receptor subunit in the presence of one or more additional receptor subunits and wherein the receptor subunit is capable of undergoing a conformational reorganization induced by ligand binding when multimerized;
   wherein the conformational reorganization inhibits the intracellular signaling domains of each receptor subunit;
   wherein the scaffold domain comprises an extracellular domain and a transmembrane domain;
   wherein the extracellular domain is operably linked to a ligand binding domain; and
   wherein the transmembrane domain is operably linked to an intracellular signaling domain.
403. The isolated polynucleotide or set of isolated polynucleotides of claim 402, further comprising one or more nucleic acid sequences, wherein each additional nucleic acid sequence encoding an additional chimeric ligand receptor subunit;
   wherein each additional receptor subunit comprises a scaffold domain;
   wherein the scaffold domain of each additional receptor subunit comprises an extracellular domain and a transmembrane domain;
   wherein the extracellular domain of each additional receptor subunit is operably linked to a ligand binding domain; and
   wherein the transmembrane domain of each additional receptor subunit is operably linked to an intracellular signaling domain.
   404. The isolated polynucleotide or set of isolated polynucleotides of claim 402 or claim 403, wherein the receptor subunits multimerize via their scaffold domains to form the chimeric ligand receptor.
   405. The isolated polynucleotide or set of isolated polynucleotides of any one of claims 402-404, wherein the multimerized receptor subunits undergo a conformational reorganization upon ligand binding to the chimeric receptor, and wherein the conformational reorganization inhibits the intracellular signaling domains of each receptor subunit.
406. The isolated polynucleotide or set of isolated polynucleotides of any one of claims 402-405, wherein the chimeric ligand binding domains of each receptor subunit bind the same ligand.
407. An isolated polynucleotide or a set of isolated polynucleotides comprising two or more nucleic acid sequences, wherein each nucleic acid sequence encoding a chimeric ligand receptor subunit;
   wherein each receptor subunit comprises a scaffold domain;
   wherein the receptor subunits multimerize via their scaffold domains to form the chimeric ligand receptor;
   wherein each scaffold domain comprises an extracellular domain and a transmembrane domain;
   wherein the extracellular domain is operably linked to a ligand binding domain, wherein the ligand binding domains of each receptor subunit bind the same ligand;
   wherein the transmembrane domain is operably linked to an intracellular signaling domain;
   wherein binding of the ligand induces a conformational reorganization of the multimerized receptor subunits; and
   wherein the conformational reorganization inhibits the intracellular signaling domains of each receptor subunit.
408. The isolated polynucleotide or set of isolated polynucleotides of any one of claims 404-407, wherein multimerization of the receptor subunits occurs prior to ligand binding.
409. The isolated polynucleotide or set of isolated polynucleotides of any one of claims 404-408, wherein the multimerized receptor subunits comprise a dimer, a trimer, tetramer, pentamer, or hexamer.
410. The isolated polynucleotide or set of isolated polynucleotides of any one of claims 404-409, wherein the multimerized receptor subunits comprise a dimer.
411. The isolated polynucleotide or set of isolated polynucleotides of any one of claims 405-410, wherein the conformational reorganization comprises a rotation of each scaffold domain around its own axis.
412. The isolated polynucleotide or set of isolated polynucleotides of any one of claims 405-411, wherein the scaffold domain comprises the extracellular domain and transmembrane domain of a cytokine receptor.
413. The isolated polynucleotide or set of isolated polynucleotides of any one of claims 402-412, wherein the scaffold domain is inert to its native ligand.
414. The isolated polynucleotide or set of isolated polynucleotides of any one of claims 402-412, wherein the scaffold domain comprises the extracellular domain and transmembrane domain of an erythropoietin receptor (EpoR).
415. The isolated polynucleotide or set of isolated polynucleotides of claim 414 wherein the scaffold domain is inert to erythropoietin.
416. The isolated polynucleotide or set of isolated polynucleotides of claim 414 or claim 415, wherein the scaffold domain comprises an extracellular domain and transmembrane domain comprising an amino acid sequence having 90% or greater sequence identity to SEQ ID NO: 8.
417. The isolated polynucleotide or set of isolated polynucleotides of any one of claims 402-412, wherein the scaffold domain comprises the extracellular domain and transmembrane domain of a protein selected from the group consisting of a leptin receptor (Ob-R), a growth hormone receptor (GHR), an epidermal growth factor receptor (EGFR), Trk-B and p75^{NTR}.
418. The isolated polynucleotide or set of isolated polynucleotides of claim 417, wherein the scaffold domain is inert to leptin, growth hormone, epidermal growth factor (EGF), or brain-derived neurotrophic factor (BDNF).
419. The isolated polynucleotide or set of isolated polynucleotides of claim 417 or claim 418, wherein the scaffold domain comprises an extracellular domain and transmembrane domain comprising an amino acid sequence having 90% or greater sequence identity to a sequence selected from the group consisting of SEQ ID NO: 12, SEQ ID NO: 16, SEQ ID NO: 20, SEQ ID NO: 24, and SEQ ID NO: 28.
420. The isolated polynucleotide or set of isolated polynucleotides of any one of claims 402-419, wherein the extracellular domain, the transmembrane domain, or both the extracellular domain and transmembrane domain of the scaffold domain comprise one or more modifications.
421. The isolated polynucleotide or set of isolated polynucleotides of any one of claims 402-420, wherein the extracellular domain comprises one or more amino acid substitutions, optionally wherein the extracellular domain comprises an F93A amino acid substitution.
422. The isolated polynucleotide or set of isolated polynucleotides of any one of claims 402-421, wherein one or more additional amino acid residues are inserted adjacent to the transmembrane domain.
423. The isolated polynucleotide or set of isolated polynucleotides of any one of claims 402-421, wherein one or more additional amino acid residues are inserted within the transmembrane domain.
424. The isolated polynucleotide or set of isolated polynucleotides of claim 422 or claim 423, wherein the one or more additional amino acid residues are alanine residues.
425. The isolated polynucleotide or set of isolated polynucleotides of claim 424, wherein the transmembrane domain further comprises one, two, three, or four additional alanine residues.
426. The isolated polynucleotide or set of isolated polynucleotides of any one of claims 423-425, wherein the one or more additional amino acid residues are inserted C-terminal to the transmembrane domain.
427. The isolated polynucleotide or set of isolated polynucleotides of any one of claims 402-426, wherein the ligand binding domain is linked to the extracellular domain through an extracellular linker region.
428. The isolated polynucleotide or set of isolated polynucleotides of claim 427, wherein the extracellular linker region comprises one or more amino acid residues, optionally wherein the one or more amino acid residues comprise amino acids residues Serine-Glycine-Glutamic acid-Phenylalanine.
429. The isolated polynucleotide or set of isolated polynucleotides of any one of claims 402-428, wherein the ligand binding domain does not bind a native ligand of the scaffold domain.
430. The isolated polynucleotide or set of isolated polynucleotides of any one of claims 402-428, wherein the ligand binding domain does not bind erythropoietin.
431. The isolated polynucleotide or set of isolated polynucleotides of any one of claims 402-428, wherein the ligand binding domain does not bind a ligand selected from the group consisting of leptin, growth hormone, epidermal growth factor (EGF), and brain-derived neurotrophic factor (BDNF).
432. The isolated polynucleotide or set of isolated polynucleotides of any one of claims 402-428, wherein the ligand binding domain is not derived from a cytokine receptor.
433. The isolated polynucleotide or set of isolated polynucleotides of any one of claims 402-432, wherein the ligand binding domain binds to a ligand selected from the group consisting of a protein complex, a protein, a peptide, a nucleic acid, a small molecule, and a chemical agent.
434. The isolated polynucleotide or set of isolated polynucleotides of claim 433 wherein the ligand is selected from the group consisting of an antigen, a cytokine, a survival factor, a chemokine, a hormone, a transmitter, a growth factor, extracellular matrix, and a death factor.
435. The isolated polynucleotide or set of isolated polynucleotides of any one of claims 402-434, wherein the ligand binding domain binds to a ligand displayed on a surface or expressed on a cell surface.
436. The isolated polynucleotide or set of isolated polynucleotides of claim 435, wherein the the ligand expressed on a cell surface is a tumor-associated antigen.
437. The isolated polynucleotide or set of isolated polynucleotides of claim 436, wherein the tumor-associated antigen is selected from the group consisting of MS4A3, VSTM1, LAT2, MLC1, CD131, GAPT, PRAM1, SLC22A16, SLC17A9, SPNS3, FLT3, CD33, CLEC12A, ADGRE2, IL3RA, CD117, CD93, IL1RAP, CD244, CCR1, LILRB2, PIEZO1, CD38, EMB, MYADM, LILRA2, CD300LF, CD70, 5T4, ADAM9, AFP, AXL, B7-H3, B7-H4, B7-H6, C4.4, CA6, Cadherin 3, Cadherin 6, CCR4, CD123, CD133, CD138, CD142, CD166, CD25, CD30, CD352, CD37, CD38, CD44, CD56, CD66e, CD71, CD74, CD79b, CD80, CEA, CEACAM5, Claudin18.2, cMet, CSPG4, CTLA, DLK1, DLL3, DR5, EGFR, ENPP3, EpCAM, EphA2, Ephrin A4, ETBR, FGFR2, FGFR3, FRalpha, FRb, GCC, GD2, GFRa4, gpA33, GPC3, gpNBM, GPRC5, HER2, IL-13R, IL-13Ra2, IL-8, IL-15, IL1RAP, Integrin aV, KIT, L1CAM, LAMP1, Lewis Y, LeY, LIV-1, LRRC, LY6E, MCSP, Mesothelin, MUC1, MUC16, MUC1C, NaPi2B, Nectin 4, NKG2D, NOTCH3, NY ESO 1, Ovarin, P-cadherin, pan-Erb2, PSCA, PSMA, PTK7, ROR1, S Aures, SCT, SLAMF7, SLITRK6, SSTR2, STEAP1, Survivin, TDGF1, TIM1, TROP2, and WT1.
438. The isolated polynucleotide or set of isolated polynucleotides of any one of claims 402-434, wherein the ligand binding domain binds to a soluble ligand.
439. The isolated polynucleotide or set of isolated polynucleotides of claim 438, wherein the soluble ligand is a secreted cytokine.
440. The isolated polynucleotide or set of isolated polynucleotides of claim 439, wherein the secreted cytokine is an immunosuppressive cytokine.
441. The isolated polynucleotide or set of isolated polynucleotides of claim 439, wherein the secreted cytokine is selected from the group consisting of TGFbeta, IL-10, IL-6, IL-23, IL-8, CCL2, CXCL12, CXCL8, CXCL1, CXCL2, CXCL3, CXCL8, CXCL13, CCL5, CCL17, and CCL22.
442. The isolated polynucleotide or set of isolated polynucleotides of any one of claims 402-441, wherein the ligand binding domain binds to an antigen.
443. The isolated polynucleotide or set of isolated polynucleotides of claim 442, wherein the ligand binding domain comprises an antibody, or antigen-binding fragment thereof.
444. The isolated polynucleotide or set of isolated polynucleotides of claim 443, wherein the ligand binding domain comprises a single chain variable fragment (scFv), or a single-domain antibody (sdAb).
445. The isolated polynucleotide or set of isolated polynucleotides of any one of claims 402-443, wherein each of the ligand binding domains comprises a single chain variable fragment (scFv), optionally wherein each scFv specifically binds to a distinct epitope of the antigen.
446. The isolated polynucleotide or set of isolated polynucleotides of any one of claims 403-445, wherein the ligand binding domains of each receptor subunit are distinct from one another.
447. The isolated polynucleotide or set of isolated polynucleotides of claim 446, wherein the chimeric ligand receptor comprises two ligand binding domains, and wherein one ligand binding domain comprises an immunoglobulin heavy chain variable domain (V_{H}) and the second ligand binding domain comprises an immunoglobulin light chain variable domain (V_{L}).
448. The isolated polynucleotide or set of isolated polynucleotides of any one of 403-445, wherein the ligand binding domains of each receptor subunit are the same as one another.
449 The isolated polynucleotide or set of isolated polynucleotides of claim 443, wherein the antibody, or antigen-binding fragment thereof, is a single-domain VHH camelid antibody.
450. The isolated polynucleotide or set of isolated polynucleotides of any one of claims 402-449, wherein the intracellular signaling domain is inert to native ligand binding of the scaffold domain.
451. The isolated polynucleotide or set of isolated polynucleotides of any one of claims 402-450, wherein the intracellular signaling domain does not comprise an endogenous intracellular signaling domain of the scaffold domain.
452. The isolated polynucleotide or set of isolated polynucleotides of any one of claims 402-450, wherein the intracellular signaling domain does not comprise an endogenous erythropoietin receptor (EpoR) intracellular signaling domain.
453. The isolated polynucleotide or set of isolated polynucleotides of any one of claims 402-450, wherein the intracellular signaling domain does not comprise an intracellular signaling domain selected from the group consisting of an endogenous leptin receptor (Ob-R) intracellular signaling domain, an endogenous growth hormone receptor (GHR) intracellular signaling domain, an endogenous epidermal growth factor receptor (EGFR) intracellular signaling domain, an endogenous Trk-B intracellular signaling domain, and an endogenous p75^{NTR} intracellular signaling domain.
454. The isolated polynucleotide or set of isolated polynucleotides of any one of claims 402-450, wherein the intracellular signaling domain does not comprise an endogenous cytokine receptor intracellular signaling domain.
455 The isolated polynucleotide or set of isolated polynucleotides of any one of claims 402-454, wherein the intracellular signaling domain induces downstream signaling via a JAK/STAT (Janus kinase/signal transducer and activator of transcription) signaling pathway, a MAPK (mitogen-activated protein kinase) signaling pathway, a PLCG (phospholipase C gamma) signaling pathway, or a PI3K/Akt (phosphatidylinositol 3-kinase/protein kinase B) signaling pathway.
456. The isolated polynucleotide or set of isolated polynucleotides of any one of claims 402-454, wherein the intracellular signaling domain is selected from the group consisting of an intracellular signal transduction domain of IL-6RB (interleukin 6 receptor B), an intracellular signal transduction domain of FGFR1 (fibroblast growth factor receptor 1), and an intracellular signal transduction domain of VEGFR2 (vascular endothelial growth factor receptor 2).
457. The isolated polynucleotide or set of isolated polynucleotides of any one of claims 402-454, wherein the intracellular signaling domain is an intracellular signal transduction domain of IL-6KB and induces downstream signaling via the JAK/STAT signaling pathway.
458. The isolated polynucleotide or set of isolated polynucleotides of any one of claims 402-454, wherein the intracellular signaling domain is an intracellular signal transduction domain derived from an IL-2 signaling pathway, an IL-7 signaling pathway, or an IL-15 signaling pathway.
459. The isolated polynucleotide or set of isolated polynucleotides of any one of claims 402-454, wherein the intracellular signaling domain is an intracellular signal transduction domain of FGFR1 and induces downstream signaling via the MAPK signaling pathway.
460. The isolated polynucleotide or set of isolated polynucleotides of any one of claims 402-454, wherein the intracellular signaling domain is an intracellular signal transduction domain of VEGFR2 and induces downstream signaling via the PLCG signaling pathway.
461. The isolated polynucleotide or set of isolated polynucleotides of any one of claims 402-454, wherein the intracellular signaling domain is an intracellular signal transduction domain of VEGFR2 and induces downstream signaling via the PI3K/Akt signaling pathways.
462. The isolated polynucleotide or set of isolated polynucleotides of any one of claims 402-461, wherein the intracellular signaling domain comprises one or more modifications that modulate signaling activity of the intracellular signaling domain, optionally wherein the one or more modifications are one or more amino acid substitutions.
463. A vector or a set of vectors comprising the polynucleotide or set of polynucleotides of any one of claims 400-462.
464. A genetically engineered cell comprising the polynucleotide or set of polynucleotides of any one of claims 400-462 or the vector or set of vectors of claim 463.
465. A genetically engineered cell expressing the chimeric ligand receptor of any one of claims 340-399.
466. The genetically engineered cell of claim 464 or claim 465, wherein the cell further comprises an engineered transgene, wherein the transgene comprises a synthetic promoter operably linked to a polynucleotide comprising a nucleic acid sequence encoding a target product.
467. The genetically engineered cell of claim 466, wherein the synthetic promoter is responsive to intracellular signaling from the chimeric ligand receptor.
468. The genetically engineered cell of claim 466 or claim 467, wherein the target product is selected from the group consisting of a therapeutic molecule, a prophylactic molecule, and a diagnostic molecule.
469. The genetically engineered cell of any one of claims 466-468, wherein the target product is a chimeric antigen receptor (CAR).
470. The genetically engineered cell of any one of claims 466-468, wherein the target product is a proinflammatory cytokine.
471. The genetically engineered cell of claim 470, wherein the proinflammatory cytokine is selected from the group consisting of IL-2, IL-7, IL-12, IL-15, IL-18, and IL-21.
472. The genetically engineered cell of any one of claims 464-471, wherein the cell further expresses one or more additional chimeric ligand receptors.
473. The genetically engineered cell of claim 472, wherein the chimeric ligand receptors are each distinct from one another.
474. The genetically engineered cell of claim 473, wherein the ligand binding domains of each chimeric ligand receptor bind a different ligand.
475. A genetically engineered cell expressing two or more chimeric ligand receptors of any one of claims 340-399.
476. The genetically engineered cell of claim 475, wherein the chimeric ligand receptors are each distinct from one another.
477. The genetically engineered cell of claim 476, wherein the ligand binding domains of each chimeric ligand receptor bind a different ligand.
478. The genetically engineered cell of any one of claims 472-477, wherein the cell further comprises two or more engineered transgenes, wherein each transgene comprises a synthetic promoter operably linked to a polynucleotide comprising a nucleic acid sequence encoding a target product.
479. The genetically engineered cell of claim 478, wherein each synthetic promoter is responsive to intracellular signaling from a distinct chimeric ligand receptor from the two or more chimeric ligand receptors expressed on the cell.
480. The genetically engineered cell of claim 478 or claim 479 wherein each target product is independently selected from the group consisting of a therapeutic molecule, a prophylactic molecule, and a diagnostic molecule.
481. The genetically engineered cell of any one of claims 464-480, wherein the cell is a mammalian cell.
482. The genetically engineered cell of claim 481, wherein the mammalian cell is a stem cell or a neuronal cell.
483. The genetically engineered cell of claim 482, wherein the stem cell is selected from the group consisting of an adult stem cell, an iPS cell, a bone marrow stem cell, a peripheral blood stem cell, and a mesenchymal stem cell (MSC).
484. The genetically engineered cell of claim 481, wherein the mammalian cell is an immune cell.
485. The genetically engineered cell of claim 484, wherein the immune cell is selected from the group consisting of a T cell, a B cell, an NK cell, a monocyte, a macrophage, an innate lymphoid cell, a mast cell, an eosinophil, a basophil, a neutrophil, and a dendritic cell.
486. The genetically engineered cell of claim 484, wherein the immune cell is a T cell.
487. The genetically engineered cell of claim 486, wherein the T cell is selected from the group consisting of a helper T cell, a cytotoxic T cell, a memory T cell, a regulatory T cell, a natural killer T cell, and a gamma delta T cell.
488. A method comprising contacting the chimeric ligand receptor of any one of claims 340-399 or the genetically engineered cell of any one of claims 464-487 with a biological tissue or biological fluid.
489. The method of claim 488, wherein the biological tissue or biological fluid is in a subject or is obtained from a subject.
490. The method of claim 489, wherein the subject has been diagnosed with, is at risk of developing, or is suspected of having a medical condition, optionally wherein the medical condition is a cancer or inflammatory condition.
491. A method of activating a signaling pathway, comprising:
   contacting the chimeric ligand receptor of any one of claims 340-399 or the genetically engineered cell of any one of claims 464-487 with a cognate ligand under conditions suitable for the chimeric ligand receptor to bind the cognate ligand,
   wherein binding of the cognate ligand with the chimeric ligand receptor induces a conformational reorganization of the multimerized scaffold domains that activates the intracellular signaling domains.
492. The method of claim 491, further comprising administering the cognate ligand to a surface of a cell.
493. A method of producing a genetically engineered cell expressing a chimeric ligand receptor, comprising:
   transfecting the isolated polynucleotide or set of isolated polynucleotides of any one of claims 400-462 or the vector or set of vectors of claim 463 into a cell; and
   inducing expression of the chimeric ligand receptor in the cell.
494. The method of claim 493, further comprising:
   transfecting into the cell an isolated polynucleotide comprising a synthetic promoter operably linked to a nucleic acid sequence encoding a target product.
495. The method of claim 494 Wherein the synthetic promoter is responsive to intracellular signaling from the chimeric ligand receptor.
496. The method of any one of claims 488-495, wherein inducing expression of the chimeric ligand receptor comprises culturing the cell under conditions suitable for the cell to express the chimeric ligand receptor on a cell membrane of the cell.
497. A method comprising transfecting a cell with the polynucleotide or set of isolated polynucleotides of any one of claims 400-462 or the vector or set of vectors of claim 463, optionally wherein the cell is a mammalian cell, and optionally wherein the mammalian cell is an immune cell.
498. A kit comprising a polynucleotide comprising a nucleic acid sequence encoding at least one chimeric ligand receptor of any one of claims 340-399.
499. The kit of claim 498, further comprising at least one engineered transgene comprising a synthetic promoter operably linked to a polynucleotide comprising a nucleic acid sequence encoding a target product.
500. A genetically engineered cell expressing a chimeric ligand receptor, wherein the chimeric ligand receptor comprises two or more receptor subunits,
   wherein each receptor subunit comprises a scaffold domain;
   wherein the receptor subunits multimerize via their scaffold domains to form the chimeric ligand receptor;
   wherein each scaffold domain comprises an extracellular domain and a transmembrane domain;
   wherein the extracellular domain is operably linked to a ligand binding domain, wherein the ligand binding domains of each receptor subunit bind the same ligand;
   wherein the transmembrane domain is operably linked to an intracellular signaling domain;
   wherein binding of the ligand induces a conformational reorganization of the multimerized receptor subunits;
   wherein the cell further comprises an engineered transgene, wherein the transgene comprises a synthetic promoter operably linked to a polynucleotide comprising a nucleic acid sequence encoding a chimeric antigen receptor (CAR) or an engineered T-cell receptor (TCR).
501. The genetically engineered cell of claim 500, wherein multimerization of the receptor subunits occurs prior to ligand binding.
502. The genetically engineered cell of claim 500 or claim 501 wherein the multimerized receptor subunits comprise a dimer, a trimer, tetramer, pentamer, or hexamer.
503. The genetically engineered cell of any one of claims 500-502, wherein the multimerized receptor subunits comprise a dimer.
504. The genetically engineered cell of any one of claims 500-503, wherein the conformational reorganization comprises a rotation of each scaffold domain around its own axis.
505. The genetically engineered cell of any one of claims 500-504, wherein the conformational reorganization activates the intracellular signaling domains of each receptor subunit.
506. The genetically engineered cell of any one of claims 500-504, wherein the conformational reorganization inhibits the intracellular signaling domains of each receptor subunit.
507. The genetically engineered cell of any one of claims 500-506, wherein the scaffold domain comprises the extracellular domain and transmembrane domain of a cytokine receptor.
508. The genetically engineered cell of any one of claims 500-507, wherein the scaffold domain is inert to its native ligand.
509. The genetically engineered cell of any one of claims 500-507, wherein the scaffold domain comprises the extracellular domain and transmembrane domain of an erythropoietin receptor (EpoR).
510. The genetically engineered cell of claim 509, wherein the scaffold domain is inert to erythropoietin.
511. The genetically engineered cell of claim 509 or claim 510, wherein the scaffold domain comprises an extracellular domain and transmembrane domain comprising an amino acid sequence having 90% or greater sequence identity to SEQ ID NO: 8.
512. The genetically engineered cell of any one of claims 500-507, wherein the scaffold domain comprises the extracellular domain and transmembrane domain of a protein selected from the group consisting of a leptin receptor (Ob-R), a growth hormone receptor (GHR), an epidermal growth factor receptor (EGFR), Trk-B and p75^{NTR}.
513. The genetically engineered cell of claim 512, wherein the scaffold domain is inert to leptin, growth hormone, epidermal growth factor (EGF), or brain-derived neurotrophic factor (BDNF).
514. The genetically engineered cell of claim 512 or claim 513, wherein the scaffold domain comprises an extracellular domain and transmembrane domain comprising an amino acid sequence having 90% or greater sequence identity to a sequence selected from the group consisting of SEQ ID NO: 12, SEQ ID NO: 16, SEQ ID NO: 20, SEQ ID NO: 24, and SEQ ID NO: 28.
515. The genetically engineered cell of any one of claims 500-514, wherein the extracellular domain, the transmembrane domain, or both the extracellular domain and transmembrane domain of the scaffold domain comprise one or more modifications.
516. The genetically engineered cell of claim 500-515, wherein the extracellular domain comprises one or more amino acid substitutions, optionally wherein the extracellular domain comprises an F93A amino acid substitution.
517. The genetically engineered cell of any one of claims 500-516, wherein one or more additional amino acid residues are inserted adjacent to the transmembrane domain.
518. The genetically engineered cell of any one of claims 500-516, wherein one or more additional amino acid residues are inserted within the transmembrane domain.
519. The genetically engineered cell of claim 517 or claim 518, wherein the one or more additional amino acid residues are alanine residues.
520. The genetically engineered cell of claim 519, wherein the transmembrane domain further comprises one, two, three, or four additional alanine residues.
521. The genetically engineered cell of any one of claims 517-520, wherein the one or more additional amino acid residues are inserted C-terminal to the transmembrane domain.
522. The genetically engineered cell of any one of claims 500-521, wherein the ligand binding domain is linked to the extracellular domain through an extracellular linker region.
523. The genetically engineered cell of claim 522, wherein the extracellular linker region comprises one or more amino acid residues, optionally wherein the one or more amino acid residues comprise amino acids residues Serine-Glycine-Glutamic acid-Phenylalanine.
524. The genetically engineered cell of any one of claims 500-523, wherein the ligand binding domain does not bind a native ligand of the scaffold domain.
525. The genetically engineered cell of any one of claims 500-524, wherein the ligand binding domain does not bind erythropoietin.
526. The genetically engineered cell of any one of claims 500-524, wherein the ligand binding domain does not bind a ligand selected from the group consisting of leptin, growth hormone, epidermal growth factor (EGO, and brain-derived neurotrophic factor (BDNF).
527. The genetically engineered cell of any one of claims 500-524, wherein the ligand binding domain is not derived from a cytokine receptor.
528. The genetically engineered cell of any one of claims 500-527, wherein the ligand binding domain binds to a ligand selected from the group consisting of a protein complex, a protein, a peptide, a nucleic acid, a small molecule, and a chemical agent.
529. The genetically engineered cell of claim 528, wherein the ligand is selected from the group consisting of an antigen, a cytokine, a survival factor, a chemokine, a hormone, a transmitter, a growth factor, extracellular matrix, and a death factor.
530. The genetically engineered cell of any one of claims 500-529, wherein the ligand binding domain binds to a ligand displayed on a surface or expressed on a cell surface.
531. The genetically engineered cell of claim 530, wherein the the ligand expressed on a cell surface is a tumor-associated antigen.
532. The genetically engineered cell of claim 531, wherein the tumor-associated antigen is selected from the group consisting of MS4A3, VSTM1, LAT2, MLC1, CD131, GAPT, PRAM1, SLC22A16, SLC17A9, SPNS3, FLT3, CD33, CLEC12A, ADGRE2, IL3RA, CD117, CD93, IL1RAP, CD244, CCR1, LILRB2, PIEZO1, CD38, EMB, MYADM, LILRA2, CD300LF, CD70, 5T4, ADAM9, AFP, AXL, B7-H3, B7-H4, B7-H6, C4.4, CA6, Cadherin 3, Cadherin 6, CCR4, CD123, CD133, CD138, CD142, CD166, CD25, CD30, CD352, CD37, CD38, CD44, CD56, CD66e, CD71, CD74, CD79b, CD80, CEA, CEACAM5, Claudin18.2, cMet, CSPG4, CTLA, DLK1, DLL3, DR5, EGFR, ENPP3, EpCAM, EphA2, Ephrin A4, ETBR, FGFR2, FGFR3, FRalpha, FRb, GCC, GD2, GFRa4, gpA33, GPC3, gpNBM, GPRC5, HER2, IL-13R, IL-3Ra2, IL-8, IL-15, IL1RAP, Integrin aV, KIT, L1CAM, LAMP1, Lewis Y, LeY, LIV-1, LRRC, LY6E, MCSP, Mesothelin, MUC1, MUC16, MUC1C, NaPi2B, Nectin 4, NKG2D, NOTCH3, NY ESO 1, Ovarin, P-cadherin, pan-Erb2, PSCA, PSMA, PTK7, ROR1, S Aures, SCT, SLAMF7, SLITRK6, SSTR2, STEAP1, Survivin, TDGF1, TIM1, TROP2, and WT1.
533. The genetically engineered cell of any one of claims 500-529, wherein the ligand binding domain binds to a soluble ligand.
534. The genetically engineered cell of claim 533, wherein the soluble ligand is a secreted cytokine.
535. The genetically engineered cell of claim 534 wherein the secreted cytokine is an immunosuppressive cytokine.
536. The genetically engineered cell of claim 534, wherein the secreted cytokine is selected from the group consisting of TGFbeta, IL-10, IL-6, IL-23, IL-8, CCL2, CXCL12, CXCL8, CXCL1, CXCL2, CXCL3, CXCL8, CXCL13, CCL5, CCL17, and CCL22.
537. The genetically engineered cell of any one of claims 500-536, wherein the ligand binding domain binds to an antigen.
538. The genetically engineered cell of claim 537, wherein the ligand binding domain comprises an antibody, or antigen-binding fragment thereof.
539. The genetically engineered cell of claim 538, wherein the ligand binding domain comprises a single chain variable fragment (scFv), or a single-domain antibody (sdAb).
540. The genetically engineered cell of any one of claims 500-538, wherein each of the ligand binding domains comprises a single chain variable fragment (scFv), optionally wherein each scFv specifically binds to a distinct epitope of the antigen.
541. The genetically engineered cell of any one of claims 500-540, wherein the ligand binding domains of each receptor subunit are distinct from one another.
542. The genetically engineered cell of claim 541, wherein the chimeric ligand receptor comprises two ligand binding domains, and wherein one ligand binding domain comprises an immunoglobulin heavy chain variable domain (V_{H}) and the second ligand binding domain comprises an immunoglobulin light chain variable domain (V_{L}).
543. The genetically engineered cell of any one of claims 500-540, wherein the ligand binding domains of each receptor subunit are the same as one another.
544. The genetically engineered cell of claim 537, wherein the antibody, or antigen-binding fragment thereof, is a single-domain VHH camelid antibody.
545. The genetically engineered cell of any one of claims 500-544, wherein the intracellular signaling domain is inert to native ligand binding of the scaffold domain.
546. The genetically engineered cell of any one of claims 500-545, wherein the intracellular signaling domain does not comprise an endogenous intracellular signaling domain of the scaffold domain.
547. The genetically engineered cell of any one of claims 500-545, wherein the intracellular signaling domain does not comprise an endogenous erythropoietin receptor (EpoR) intracellular signaling domain.
548. The genetically engineered cell of any one of claims 500-545, wherein the intracellular signaling domain does not comprise an intracellular signaling domain selected from the group consisting of an endogenous leptin receptor (Ob-R) intracellular signaling domain, an endogenous growth hormone receptor (GHR) intracellular signaling domain, an endogenous epidermal growth factor receptor (EGFR) intracellular signaling domain, an endogenous Trk-B intracellular signaling domain, and an endogenous p75^{NTR} intracellular signaling domain.
549. The genetically engineered cell of any one of claims 500-545, wherein the intracellular signaling domain does not comprise an endogenous cytokine receptor intracellular signaling domain.
550. The genetically engineered cell of any one of claims 500-549, wherein the intracellular signaling domain induces downstream signaling via a JAK/STAT (Janus kinase/signal transducer and activator of transcription) signaling pathway, a MAPK (mitogen-activated protein kinase) signaling pathway, a PLCG (phospholipase C gamma) signaling pathway, or a PI3K/Akt (phosphatidylinositol 3-kinase/protein kinase B) signaling pathway.
551. The genetically engineered cell of any one of claims 500-549, wherein the intracellular signaling domain is selected from the group consisting of an intracellular signal transduction domain of IL-6RB (interleukin 6 receptor B), an intracellular signal transduction domain of FGFR1 (fibroblast growth factor receptor 1), and an intracellular signal transduction domain of VEGFR2 (vascular endothelial growth factor receptor 2).
552. The genetically engineered cell of any one of claims 500-549, wherein the intracellular signaling domain is an intracellular signal transduction domain of IL-6RB and induces downstream signaling via the JAK/STAT signaling pathway.
553. The genetically engineered cell of any one of claims 500-549, wherein the intracellular signaling domain is an intracellular signal transduction domain of FGFR1 and induces downstream signaling via the MAPK signaling pathway.
554. The genetically engineered cell of any one of claims 500-549, wherein the intracellular signaling domain is an intracellular signal transduction domain of VEGFR2 and induces downstream signaling via the PLCG signaling pathway.
555. The genetically engineered cell of any one of claims 500-549, wherein the intracellular signaling domain is an intracellular signal transduction domain of VEGFR2 and induces downstream signaling via the PI3K/Akt signaling pathways.
556. The genetically engineered cell of any one of claims 500-549, wherein the intracellular signaling domain is an intracellular signal transduction domain derived from an IL-2 signaling pathway, an IL-7 signaling pathway, or an IL-15 signaling pathway.
557. The genetically engineered cell of any one of claims 500-556, wherein the intracellular signaling domain comprises one or more modifications that modulate signaling activity of the intracellular signaling domain, optionally wherein the one or more modifications are one or more amino acid substitutions.
558. The genetically engineered cell of any one of claims 500-557, wherein the synthetic promoter is responsive to intracellular signaling from the chimeric ligand receptor.
559. The genetically engineered cell of any one of claims 500-558, wherein the cell further expresses one or more additional chimeric ligand receptors.
560. The genetically engineered cell of claim 559 wherein the chimeric ligand receptors are each distinct from one another.
561. The genetically engineered cell of claim 560, wherein the ligand binding domains of each chimeric ligand receptor bind a different ligand.
562. The genetically engineered cell of any one of claims 559-561, wherein the cell further comprises one or more engineered transgenes, wherein each of the one or more engineered transgenes comprises a synthetic promoter operably linked to a polynucleotide comprising a nucleic acid sequence encoding a target product.
563. The genetically engineered cell of claim 562, wherein each synthetic promoter is responsive to intracellular signaling from a distinct chimeric ligand receptor from the two or more chimeric ligand receptors expressed on the cell.
564. The genetically engineered cell of claim 562 or claim 563, wherein each target product is independently selected from the group consisting of a therapeutic molecule, a prophylactic molecule, and a diagnostic molecule.
565. The genetically engineered cell of any one of claims 500-564, wherein the cell is a mammalian cell.
566. The genetically engineered cell of claim 565, wherein the mammalian cell is a stem cell or a neuronal cell.
567. The genetically engineered cell of claim 566, wherein the stem cell is selected from the group consisting of an adult stem cell, an iPS cell, a bone marrow stem cell, a peripheral blood stem cell, and a mesenchymal stem cell (MSC).
568. The genetically engineered cell of claim 565, wherein the mammalian cell is an immune cell.
569. The genetically engineered cell of claim 568, wherein the immune cell is selected from the group consisting of a T cell, a B cell, an NK cell, a monocyte, a macrophage, an innate lymphoid cell, a mast cell, an eosinophil, a basophil, a neutrophil, and a dendritic cell.
570. The genetically engineered cell of claim 568, wherein the immune cell is a T cell.
571. The genetically engineered cell of claim 570, wherein the T cell is selected from the group consisting of a helper T cell, a cytotoxic T cell, a memory T cell, a regulatory T cell, a natural killer T cell, and a gamma delta T cell.
572. A method comprising contacting the the genetically engineered cell of any one of claims 500-571 with a biological tissue or biological fluid.
573. The method of claim 572, wherein the biological tissue or biological fluid is in a subject or is obtained from a subject.
574. The method of claim 573, wherein the subject has been diagnosed with, is at risk of developing, or is suspected of having a medical condition, optionally wherein the medical condition is a cancer or inflammatory condition.
575. A method of activating a signaling pathway, comprising:
   contacting the the genetically engineered cell of any one of claims 500-571 with a cognate ligand under conditions suitable for the chimeric ligand receptor to bind the cognate ligand,
   wherein binding of the cognate ligand with the chimeric ligand receptor induces a conformational reorganization of the multimerized scaffold domains that activates the intracellular signaling domains.
576. The method of claim 575, further comprising administering the cognate ligand to a surface of a cell.
577. A genetically engineered cell expressing a chimeric ligand receptor, wherein the chimeric ligand receptor comprises two or more receptor subunits,
   wherein each receptor subunit comprises a scaffold domain;
   wherein the receptor subunits multimerize via their scaffold domains to form the chimeric ligand receptor;
   wherein each scaffold domain comprises an extracellular domain and a transmembrane domain;
   wherein the extracellular domain is operably linked to a ligand binding domain, wherein the ligand binding domains of each receptor subunit bind the same ligand;
   wherein the transmembrane domain is operably linked to an intracellular signaling domain;
   wherein binding of the ligand induces a conformational reorganization of the multimerized receptor subunits;
   wherein the cell further comprises an engineered transgene, wherein the transgene comprises a synthetic promoter operably linked to a polynucleotide comprising a nucleic acid sequence encoding a proinflammatory cytokine.
578. The genetically engineered cell of claim 577, wherein the proinflammatory cytokine is selected from the group consisting of IL-2, IL-7, IL-12, IL-15, IL-18, and IL-21.
579. The genetically engineered cell of claim 577 or claim 578, wherein multimerization of the receptor subunits occurs prior to ligand binding.
580. The genetically engineered cell of any one of claims 577-579, wherein the multimerized receptor subunits comprise a dimer, a trimer, tetramer, pentamer, or hexamer.
581. The genetically engineered cell of any one of claims 577-580, wherein the multimerized receptor subunits comprise a dimer.
582. The genetically engineered cell of any one of claims 577-581, wherein the conformational reorganization comprises a rotation of each scaffold domain around its own axis.
583. The genetically engineered cell of any one of claims 577-582, wherein the conformational reorganization activates the intracellular signaling domains of each receptor subunit.
584. The genetically engineered cell of any one of claims 577-582, wherein the conformational reorganization inhibits the intracellular signaling domains of each receptor subunit.
585. The genetically engineered cell of any one of claims 577-584, wherein the scaffold domain comprises the extracellular domain and transmembrane domain of a cytokine receptor.
586. The genetically engineered cell of any one of claims 577-585, wherein the scaffold domain is inert to its native ligand.
587. The genetically engineered cell of any one of claims 577-585, wherein the scaffold domain comprises the extracellular domain and transmembrane domain of an erythropoietin receptor (EpoR).
588. The genetically engineered cell of claim 587, wherein the scaffold domain is inert to erythropoietin.
589. The genetically engineered cell of claim 587 or claim 588, wherein the scaffold domain comprises an extracellular domain and transmembrane domain comprising an amino acid sequence having 90% or greater sequence identity to SEQ ID NO: 8.
590. The genetically engineered cell of any one of claims 577-585, wherein the scaffold domain comprises the extracellular domain and transmembrane domain of a protein selected from the group consisting of a leptin receptor (Ob-R), a growth hormone receptor (GHR), an epidermal growth factor receptor (EGFR), Trk-B and p75^{NTR}.
591. The genetically engineered cell of claim 590, wherein the scaffold domain is inert to leptin, growth hormone, epidermal growth factor (EGF), or brain-derived neurotrophic factor (BDNF).
592. The chimeric ligand receptor of claim 590 or claim 591, wherein the scaffold domain comprises an extracellular domain and transmembrane domain comprising an amino acid sequence having 90% or greater sequence identity to a sequence selected from the group consisting of SEQ ID NO: 12, SEQ ID NO: 16, SEQ ID NO: 20, SEQ ID NO: 24, and SEQ ID NO: 28.
593. The genetically engineered cell of any one of claims 577-592, wherein the extracellular domain, the transmembrane domain, or both the extracellular domain and transmembrane domain of the scaffold domain comprise one or more modifications.
594. The genetically engineered cell of claim 577-593, wherein the extracellular domain comprises one or more amino acid substitutions, optionally wherein the extracellular domain comprises an F93A amino acid substitution.
595. The genetically engineered cell of any one of claims 577-594, wherein one or more additional amino acid residues are inserted adjacent to the transmembrane domain.
596. The genetically engineered cell of any one of claims 577-594, wherein one or more additional amino acid residues are inserted within the transmembrane domain.
597. The genetically engineered cell of claim 595 or claim 596, wherein the one or more additional amino acid residues are alanine residues.
598. The genetically engineered cell of claim 597, wherein the transmembrane domain further comprises one, two, three, or four additional alanine residues.
599. The genetically engineered cell of any one of claims 595-598, wherein the one or more additional amino acid residues are inserted C-terminal to the transmembrane domain.
600. The genetically engineered cell of any one of claims 577-599, wherein the ligand binding domain is linked to the extracellular domain through an extracellular linker region.
601. The genetically engineered cell of claim 600, wherein the extracellular linker region comprises one or more amino acid residues, optionally wherein the one or more amino acid residues comprise amino acids residues Serine-Glycine-Glutamic acid-Phenylalanine.
602. The genetically engineered cell of any one of claims 577-601, wherein the ligand binding domain does not bind a native ligand of the scaffold domain.
603. The genetically engineered cell of any one of claims 577-602, wherein the ligand binding domain does not bind erythropoietin.
604. The genetically engineered cell of any one of claims 577-602, wherein the ligand binding domain does not bind a ligand selected from the group consisting of leptin, growth hormone, epidermal growth factor (EGF), and brain-derived neurotrophic factor (BDNF).
605. The genetically engineered cell of any one of claims 577-602, wherein the ligand binding domain is not derived from a cytokine receptor.
606. The genetically engineered cell of any one of claims 577-605, wherein the ligand binding domain binds to a ligand selected from the group consisting of a protein complex, a protein, a peptide, a nucleic acid, a small molecule, and a chemical agent.
607. The genetically engineered cell of claim 606, wherein the ligand is selected from the group consisting of an antigen, a cytokine, a survival factor, a chemokine, a hormone, a transmitter, a growth factor, extracellular matrix, and a death factor.
608. The genetically engineered cell of any one of claims 577-607, wherein the ligand binding domain binds to a ligand displayed on a surface or expressed on a cell surface.
609. The genetically engineered cell of claim 608, wherein the the ligand expressed on a cell surface is a tumor-associated antigen.
610. The genetically engineered cell of claim 609, wherein the tumor-associated antigen is selected from the group consisting of MS4A3, VSTM1, LAT2, MLC1, CD131, GAPT, PRAM1, SLC22A16, SLC17A9, SPNS3, FLT3, CD33, CLEC12A, ADGRE2, IL3RA, CD117, CD93, IL1RAP, CD244, CCR1, LILRB2, PIEZO1, CD38, EMB, MYADM, LILRA2, CD300LF, CD70, 5T4, ADAM9, AFP, AXL, B7-H3, B7-H4, B7-H6, C4.4, CA6, Cadherin 3, Cadherin 6, CCR4, CD123, CD133, CD138, CD142, CD166, CD25, CD30, CD352, CD37, CD38, CD44, CD56, CD66e, CD71, CD74, CD79b, CD80, CEA, CEACAM5, Claudin18.2, cMet, CSPG4, CTLA, DLK1, DLL3, DR5, EGFR, ENPP3, EpCAM, EphA2, Ephrin A4, ETBR, FGFR2, FGFR3, FRalpha, FRb, GCC, GD2, GFRa4, gpA33, GPC3, gpNBM, GPRC5, HER2, IL-13R, IL-13Ra2, IL-8, IL-15, IL1RAP, Integrin aV, KIT, L1CAM, LAMP1, Lewis Y, LeY, LIV-1, LRRC, LY6E, MCSP, Mesothelin, MUC1, MUC16, MUC1C, NaPi2B, Nectin 4, NKG2D, NOTCH3, NY ESO 1, Ovarin, P-cadherin, pan-Erb2, PSCA, PSMA, PTK7, ROR1, S Aures, SCT, SLAMF7, SLITRK6, SSTR2, STEAP1, Survivin, TDGF1, TIM1, TROP2, and WT1.
611. The genetically engineered cell of any one of claims 577-607, wherein the ligand binding domain binds to a soluble ligand.
612. The genetically engineered cell of claim 611, wherein the soluble ligand is a secreted cytokine.
613. The genetically engineered cell of claim 612, wherein the secreted cytokine is an immunosuppressive cytokine.
614. The genetically engineered cell of claim 612, wherein the secreted cytokine is selected from the group consisting of TGFbeta, IL-10, IL-6, IL-23, IL-8, CCL2, CXCL12, CXCL8, CXCL1, CXCL2, CXCL3, CXCL8, CXCL13, CCL5, CCL17, and CCL22.
615. The genetically engineered cell of any one of claims 577-614, wherein the ligand binding domain binds to an antigen.
616. The genetically engineered cell of claim 615, wherein the ligand binding domain comprises an antibody, or antigen-binding fragment thereof.
617. The genetically engineered cell of claim 616, wherein the ligand binding domain comprises a single chain variable fragment (scFv), or a single-domain antibody (sdAb).
618. The genetically engineered cell of any one of claims 577-616, wherein each of the ligand binding domains comprises a single chain variable fragment (scFv), optionally wherein each scFv specifically binds to a distinct epitope of the antigen.
619. The genetically engineered cell of any one of claims 577-618, wherein the ligand binding domains of each receptor subunit are distinct from one another.
620. The genetically engineered cell of claim 619, wherein the chimeric ligand receptor comprises two ligand binding domains, and wherein one ligand binding domain comprises an immunoglobulin heavy chain variable domain (V_{H}) and the second ligand binding domain comprises an immunoglobulin light chain variable domain (V_{L}).
621. The genetically engineered cell of any one of claims 577-618, wherein the ligand binding domains of each receptor subunit are the same as one another.
622. The genetically engineered cell of claim 616, wherein the antibody, or antigen-binding fragment thereof, is a single-domain VHH camelid antibody.
623. The genetically engineered cell of any one of claims 577-622, wherein the intracellular signaling domain is inert to native ligand binding of the scaffold domain.
624. The genetically engineered cell of any one of claims 577-623, wherein the intracellular signaling domain does not comprise an endogenous intracellular signaling domain of the scaffold domain.
625. The genetically engineered cell of any one of claims 577-623, wherein the intracellular signaling domain does not comprise an endogenous erythropoietin receptor (EpoR) intracellular signaling domain.
626. The genetically engineered cell of any one of claims 577-623, wherein the intracellular signaling domain does not comprise an intracellular signaling domain selected from the group consisting of an endogenous leptin receptor (Ob-R) intracellular signaling domain, an endogenous growth hormone receptor (GHR) intracellular signaling domain, an endogenous epidermal growth factor receptor (EGFR) intracellular signaling domain, an endogenous Trk-B intracellular signaling domain, and an endogenous p75^{NTR} intracellular signaling domain.
627. The genetically engineered cell of any one of claims 577-623, wherein the intracellular signaling domain does not comprise an endogenous cytokine receptor intracellular signaling domain.
628. The genetically engineered cell of any one of claims 577-627, wherein the intracellular signaling domain induces downstream signaling via a JAK/STAT (Janus kinase/signal transducer and activator of transcription) signaling pathway, a MAPK (mitogen-activated protein kinase) signaling pathway, a PLCG (phospholipase C gamma) signaling pathway, or a PI3K/Akt (phosphatidylinositol 3-kinase/protein kinase B) signaling pathway.
629. The genetically engineered cell of any one of claims 577-627, wherein the intracellular signaling domain is selected from the group consisting of an intracellular signal transduction domain of IL-6KB (interleukin 6 receptor B), an intracellular signal transduction domain of FGFR1 (fibroblast growth factor receptor 1), and an intracellular signal transduction domain of VEGFR2 (vascular endothelial growth factor receptor 2).
630. The genetically engineered cell of any one of claims 577-627, wherein the intracellular signaling domain is an intracellular signal transduction domain of IL-6RB and induces downstream signaling via the JAK/STAT signaling pathway.
631. The genetically engineered cell of any one of claims 577-627, wherein the intracellular signaling domain is an intracellular signal transduction domain of FGFR1 and induces downstream signaling via the MAPK signaling pathway.
632. The genetically engineered cell of any one of claims 577-627, wherein the intracellular signaling domain is an intracellular signal transduction domain of VEGFR2 and induces downstream signaling via the PLCG signaling pathway.
633. The genetically engineered cell of any one of claims 577-627, wherein the intracellular signaling domain is an intracellular signal transduction domain of VEGFR2 and induces downstream signaling via the PI3K/Akt signaling pathways.
634. The genetically engineered cell of any one of claims 577-627, wherein the intracellular signaling domain is an intracellular signal transduction domain derived from an IL-2 signaling pathway, an IL-7 signaling pathway, or an IL-15 signaling pathway.
635. The genetically engineered cell of any one of claims 577-634, wherein the intracellular signaling domain comprises one or more modifications that modulate signaling activity of the intracellular signaling domain, optionally wherein the one or more modifications are one or more amino acid substitutions.
636. The genetically engineered cell of any one of claims 577-635, wherein the synthetic promoter is responsive to intracellular signaling from the chimeric ligand receptor.
637. The genetically engineered cell of any one of claims 577-636, wherein the cell further expresses one or more additional chimeric ligand receptors.
638. The genetically engineered cell of claim 637, wherein the chimeric ligand receptors are each distinct from one another.
639. The genetically engineered cell of claim 638, wherein the ligand binding domains of each chimeric ligand receptor bind a different ligand.
640. The genetically engineered cell of any one of claims 637-639, wherein the cell further comprises one or more engineered transgenes, wherein each of the one or more engineered transgenes comprises a synthetic promoter operably linked to a polynucleotide comprising a nucleic acid sequence encoding a target product.
641. The genetically engineered cell of claim 640, wherein each synthetic promoter is responsive to intracellular signaling from a distinct chimeric ligand receptor from the two or more chimeric ligand receptors expressed on the cell.
642. The genetically engineered cell of claim 640 or claim 641, wherein each target product is independently selected from the group consisting of a therapeutic molecule, a prophylactic molecule, and a diagnostic molecule.
643. The genetically engineered cell of any one of claims 577-642, wherein the cell is a mammalian cell.
644. The genetically engineered cell of claim 643, wherein the mammalian cell is a stem cell or a neuronal cell.
645. The genetically engineered cell of claim 644, wherein the stem cell is selected from the group consisting of an adult stem cell, an iPS cell, a bone marrow stem cell, a peripheral blood stem cell, and a mesenchymal stem cell (MSC).
646. The genetically engineered cell of claim 643, wherein the mammalian cell is an immune cell.
647. The genetically engineered cell of claim 646, wherein the immune cell is selected from the group consisting of a T cell, a B cell, an NK cell, a monocyte, a macrophage, an innate lymphoid cell, a mast cell, an eosinophil, a basophil, a neutrophil, and a dendritic cell.
648. The genetically engineered cell of claim 646, wherein the immune cell is a T cell.
649. The genetically engineered cell of claim 648, wherein the T cell is selected from the group consisting of a helper T cell, a cytotoxic T cell, a memory T cell, a regulatory T cell, a natural killer T cell, and a gamma delta T cell.
650. A method comprising contacting the the genetically engineered cell of any one of claims 577-649 with a biological tissue or biological fluid.
651. The method of claim 650, wherein the biological tissue or biological fluid is in a subject or is obtained from a subject.
652. The method of claim 651, wherein the subject has been diagnosed with, is at risk of developing, or is suspected of having a medical condition, optionally wherein the medical condition is a cancer or inflammatory condition.
653. A method of activating a signaling pathway, comprising:
   contacting the the genetically engineered cell of any one of claims 577-649 with a cognate ligand under conditions suitable for the chimeric ligand receptor to bind the cognate ligand,
   wherein binding of the cognate ligand with the chimeric ligand receptor induces a conformational reorganization of the multimerized scaffold domains that activates the intracellular signaling domains.
654. The method of claim 653, further comprising administering the cognate ligand to a surface of a cell.

1. A chimeric ligand receptor comprising two or more receptor subunits, wherein each receptor subunit comprises a scaffold domain;
wherein the receptor subunits multimerize via their scaffold domains to form the chimeric ligand receptor;
wherein each scaffold domain comprises an extracellular domain and a transmembrane domain of a receptor selected from the group consisting of a leptin receptor (Ob-R), a growth hormone receptor (GHR), an epidermal growth factor receptor (EGFR), Trk-B, and p75^{NTR};
wherein the extracellular domain is operably linked to a ligand binding domain,
wherein the ligand binding domains of each receptor subunit bind the same ligand;
wherein the transmembrane domain is operably linked to an intracellular signaling domain; and
wherein binding of the ligand induces a conformational reorganization of the multimerized receptor subunits,
optionally wherein multimerization of the receptor subunits occurs prior to ligand binding, optionally wherein the multimerized receptor subunits comprise a dimer, a trimer, tetramer, pentamer, or hexamer.

2. The chimeric ligand receptor of claim 1, wherein the multimerized receptor subunits comprise a dimer.

3. The chimeric ligand receptor of claim 1 or claim 2, wherein the conformational reorganization comprises a rotation of each scaffold domain around its own axis, optionally wherein the conformational reorganization activates the intracellular signaling domains of each receptor subunit or the conformational reorganization inhibits the intracellular signaling domains of each receptor subunit.

4. The chimeric ligand receptor of any one of claims 1-3, wherein:
the scaffold domain comprises the extracellular domain and transmembrane domain of a leptin receptor (Ob-R), optionally wherein the scaffold domain is inert to leptin, optionally wherein the scaffold domain comprises an extracellular domain and transmembrane domain comprising an amino acid sequence having 90% or greater sequence identity to SEQ ID NO: 12; or
the scaffold domain comprises the extracellular domain and transmembrane domain of a growth hormone receptor (GHR), optionally wherein the scaffold domain is inert to growth hormone, optionally wherein the scaffold domain comprises an extracellular domain and transmembrane domain comprising an amino acid sequence having 90% or greater sequence identity to SEQ ID NO: 16; or
the scaffold domain comprises the extracellular domain and transmembrane domain of an epidermal growth factor receptor (EGFR), optionally wherein the scaffold domain is inert to epidermal growth factor (EGF), optionally wherein the scaffold domain comprises an extracellular domain and transmembrane domain comprising an amino acid sequence having 90% or greater sequence identity to SEQ ID NO: 20; or
the scaffold domain comprises the extracellular domain and transmembrane domain of Trk-B or the wherein the scaffold domain comprises the extracellular domain and transmembrane domain of p75NTR, optionally wherein the scaffold domain is inert to brain-derived neurotrophic factor (BDNF), optionally wherein the scaffold domain comprises an extracellular domain and transmembrane domain comprising an amino acid sequence having 90% or greater sequence identity to SEQ ID NO: 24 or the scaffold domain comprises an extracellular domain and transmembrane domain comprising an amino acid sequence having 90% or greater sequence identity to SEQ ID NO: 28.

5. The chimeric ligand receptor of any one of claims 1-4, wherein the extracellular domain, the transmembrane domain, or both the extracellular domain and transmembrane domain of the scaffold domain comprise one or more modifications,
optionally wherein the extracellular domain comprises one or more amino acid substitutions,
optionally wherein one or more additional amino acid residues are inserted adjacent to the transmembrane domain,
optionally wherein one or more additional amino acid residues are inserted within the transmembrane domain,
optionally wherein the one or more additional amino acid residues are alanine residues,
optionally wherein the transmembrane domain further comprises one, two, three, or four additional alanine residues,
optionally wherein the one or more additional amino acid residues are inserted C-terminal to the transmembrane domain.

6. The chimeric ligand receptor of any one of claims 1-5, wherein the ligand binding domain is linked to the extracellular domain through an extracellular linker region,
optionally wherein the extracellular linker region comprises one or more amino acid residues, optionally wherein the one or more amino acid residues comprise amino acids residues Serine-Glycine-Glutamic acid-Phenylalanine,
optionally wherein the ligand binding domain does not bind a native ligand of the scaffold domain,
optionally wherein the ligand binding domain does not bind leptin, and/or growth hormone and/or epidermal growth factor (EGF) and/or brain-derived neurotrophic factor (BDNF),
optionally wherein the ligand binding domain binds to a ligand selected from the group consisting of a protein complex, a protein, a peptide, a nucleic acid, a small molecule, and a chemical agent,
optionally wherein the ligand binding domain binds to a ligand displayed on a surface or expressed on a cell surface or the ligand binding domain binds to a soluble ligand.

7. The chimeric ligand receptor of any one of claims 1-6, wherein:
the ligand binding domain comprises an antibody, or antigen-binding fragment thereof, optionally wherein the ligand binding domain comprises a single chain variable fragment (scFv), or a single-domain antibody (sdAb), optionally wherein each of the ligand binding domains comprises a single chain variable fragment (scFv), optionally wherein each scFv specifically binds to a distinct epitope; or
the ligand binding domains of each receptor subunit are distinct from one another, optionally wherein the chimeric ligand receptor comprises two ligand binding domains, and wherein one ligand binding domain comprises an immunoglobulin heavy chain variable domain (VH) and the second ligand binding domain comprises an immunoglobulin light chain variable domain (VL); or
the ligand binding domains of each receptor subunit are the same as one another, optionally wherein the antibody, or antigen-binding fragment thereof, is a single-domain VHH camelid antibody.

8. The chimeric ligand receptor of any one of claims 1-7, wherein the intracellular signaling domain is inert to native ligand binding of the scaffold domain,
optionally wherein the intracellular signaling domain does not comprise an endogenous intracellular signaling domain of the scaffold domain,
optionally wherein the intracellular signaling domain does not comprise an endogenous leptin receptor (Ob-R) intracellular signaling domain,
optionally wherein the intracellular signaling domain does not comprise an endogenous growth hormone receptor (GHR) intracellular signaling domain,
optionally wherein the intracellular signaling domain does not comprise an endogenous epidermal growth factor receptor (EGFR) intracellular signaling domain,
optionally wherein the intracellular signaling domain does not comprise an endogenous Trk-B intracellular signaling domain,
optionally wherein the intracellular signaling domain does not comprise an endogenous p75NTR intracellular signaling domain.

9. The chimeric ligand receptor of any one of claims 1-8, wherein:
the intracellular signaling domain induces downstream signaling via a JAK/STAT (Janus kinase/signal transducer and activator of transcription) signaling pathway, a MAPK (mitogen-activated protein kinase) signaling pathway, a PLCG (phospholipase C gamma) signaling pathway, or a PI3K/Akt (phosphatidylinositol 3-kinase/protein kinase B) signaling pathway; and/or
the intracellular signaling domain is selected from the group consisting of an intracellular signal transduction domain of IL-6RB (interleukin 6 receptor B), an intracellular signal transduction domain of FGFR1 (fibroblast growth factor receptor 1), and an intracellular signal transduction domain of VEGFR2 (vascular endothelial growth factor receptor 2).

10. The chimeric ligand receptor of any one of claims 1-9, wherein:
the intracellular signaling domain is an intracellular signal transduction domain of IL-6RB and induces downstream signaling via the JAK/STAT signaling pathway; or
the intracellular signaling domain is an intracellular signal transduction domain of FGFR1 and induces downstream signaling via the MAPK signaling pathway; or
the intracellular signaling domain is an intracellular signal transduction domain of VEGFR2 and induces downstream signaling via the PLCG signaling pathway; or
the intracellular signaling domain is an intracellular signal transduction domain of VEGFR2 and induces downstream signaling via the PI3K/Akt signaling pathways; or
the intracellular signaling domain is an intracellular signal transduction domain derived from an IL-2 signaling pathway, an IL-7 signaling pathway, or an IL-15 signaling pathway.

11. The chimeric ligand receptor of any one of claims 1-10, wherein the intracellular signaling domain comprises one or more modifications that modulate signaling activity of the intracellular signaling domain, optionally wherein the one or more modifications are one or more amino acid substitutions.

12. An isolated polynucleotide or a set of isolated polynucleotides encoding the chimeric ligand receptor of any one of claims 1-11.

13. A vector or a set of vectors comprising the polynucleotide or set of polynucleotides of claim 12.

14. A genetically engineered cell expressing the chimeric ligand receptor of any one of claims 1-11, comprising the polynucleotide or set of polynucleotides of claim 12, or comprising the vector or set of vectors of claim 13,
optionally wherein the cell further comprises an engineered transgene, wherein the transgene comprises a synthetic promoter operably linked to a polynucleotide comprising a nucleic acid sequence encoding a target product,
optionally wherein the synthetic promoter is responsive to intracellular signaling from the chimeric ligand receptor,
optionally wherein the target product is selected from the group consisting of a therapeutic molecule, a prophylactic molecule, and a diagnostic molecule,
optionally wherein the cell further expresses one or more additional chimeric ligand receptors,
optionally wherein the cell is selected from the group consisting of an adult stem cell, an iPS cell, a bone marrow stem cell, a peripheral blood stem cell, and a mesenchymal stem cell (MSC), a neuronal cell, a T cell, a B cell, an NK cell, a monocyte, a macrophage, an innate lymphoid cell, a mast cell, an eosinophil, a basophil, a neutrophil, and a dendritic cell.

15. A method comprising contacting the chimeric ligand receptor of any one of claims 1-11 or the genetically engineered cell of claim 14 with a biological tissue or biological fluid.
optionally wherein the biological tissue or biological fluid is in a subject or is obtained from a subject,
optionally wherein the subject has been diagnosed with, is at risk of developing, or is suspected of having a medical condition, optionally wherein the medical condition is a cancer or inflammatory condition.
